SEQUENCE LISTING (62 sequences)

SEQ ID NO: 1 - DNA, 15 bp, Artificial
  wildtype att recombination site
gcttttttatactaa

SEQ ID NO: 2 - DNA, 21 bp, Artificial
  recombination site
caacttttttatacaaagttg

SEQ ID NO: 3 - DNA, 25 bp, Artificial
  attB1 recombination site
agcctgcttttttgtacaaacttgt

SEQ ID NO: 4 - DNA, 233 bp, Artificial
  attP1 recombination site
tacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaagttggcattataaaaaagcattgctcatcaatttgttgcaacgaacaggtcactatcagtcaaaataaaatcattatttg

SEQ ID NO: 5 - DNA, 100 bp, Artificial
  attL1 recombination site
caaataatgattttattttgactgatagtgacctgttcgttgcaacaaattgataagcaatgcttttttataatgccaactttgtacaaaaaagcaggct

SEQ ID NO: 6 - DNA, 125 bp, Artificial
  attR1 recombination site
acaagtttgtacaaaaaagctgaacgagaaacgtaaaatgatataaatatcaatatattaaattagattttgcataaaaaacagactacataatactgtaaaacacaacatatccagtcactatg

SEQ ID NO: 7 - DNA, 27 bp, Artificial
  AttB0 recombination site
agcctgcttttttatactaacttgagc

SEQ ID NO: 8 - DNA, 27 bp, Artificial
  AttP0 recombination site
gttcagcttttttatactaagttggca

SEQ ID NO: 9 - DNA, 27 bp, Artificial
  AttL0 recombination site
agcctgcttttttatactaagttggca

SEQ ID NO: 10 - DNA, 27 bp, Artificial
  AttR0 recombination site
gttcagcttttttatactaacttgagc

SEQ ID NO: 11 - DNA, 25 bp, Artificial
  AttB1 recombination site
agcctgcttttttgtacaaacttgt

SEQ ID NO: 12 - DNA, 27 bp, Artificial
  AttP1 recombination site
gttcagcttttttgtacaaagttggca

SEQ ID NO: 13 - DNA, 27 bp, Artificial
  AttL1
agcctgcttttttgtacaaagttggca

SEQ ID NO: 14 - DNA, 25 bp, Artificial
  AttR1 recombination site
gttcagcttttttgtacaaacttgt

SEQ ID NO: 15 - DNA, 25 bp, Artificial
  AttB2 recombination site
acccagctttcttgtacaaagtggt

SEQ ID NO: 16 - DNA, 27 bp, Artificial
  AttP2 recombination site
gttcagctttcttgtacaaagttggca

SEQ ID NO: 17 - DNA, 27 bp, Artificial
  AttL2 recombination site
acccagctttcttgtacaaagttggca

SEQ ID NO: 18 - DNA, 25 bp, Artificial
  AttR2 recombination site
gttcagctttcttgtacaaagtggt

SEQ ID NO: 19 - DNA, 22 bp, Artificial
  AttB5 recombination site
caactttattatacaaagttgt

SEQ ID NO: 20 - DNA, 27 bp, Artificial
  AttP5 recombination site
gttcaactttattatacaaagttggca

SEQ ID NO: 21 - DNA, 24 bp, Artificial
  AttL5 recombination site
caactttattatacaaagttggca

SEQ ID NO: 22 - DNA, 25 bp, Artificial
  AttR5 recombination site
gttcaactttattatacaaagttgt

SEQ ID NO: 23 - DNA, 22 bp, Artificial
  AttB11 recombination site
caacttttctatacaaagttgt

SEQ ID NO: 24 - DNA, 27 bp, Artificial
  AttP11 recombination site
gttcaacttttctatacaaagttggca

SEQ ID NO: 25 - DNA, 24 bp, Artificial
  AttL11 recombination site
caacttttctatacaaagttggca

SEQ ID NO: 26 - DNA, 25 bp, Artificial
  AttR11 recombination site
gttcaacttttctatacaaagttgt

SEQ ID NO: 27 - DNA, 22 bp, Artificial
  AttB17
caacttttgtatacaaagttgt

SEQ ID NO: 28 - DNA, 27 bp, Artificial
  AttP17 recombination site
gttcaacttttgtatacaaagttggca

SEQ ID NO: 29 - DNA, 24 bp, Artificial
  AttL17 recombination site
caacttttgtatacaaagttggca

SEQ ID NO: 30 - DNA, 25 bp, Artificial
  AttR17 recombination site
gttcaacttttgtatacaaagttgt

SEQ ID NO: 31 - DNA, 22 bp, Artificial
  AttB19 recombination site
caactttttcgtacaaagttgt

SEQ ID NO: 32 - DNA, 27 bp, Artificial
  AttP19 recombination site
gttcaactttttcgtacaaagttggca

SEQ ID NO: 33 - DNA, 24 bp, Artificial
  AttL19 recombination site
caactttttcgtacaaagttggca

SEQ ID NO: 34 - DNA, 25 bp, Artificial
  AttR19 recombination site
gttcaactttttcgtacaaagttgt

SEQ ID NO: 35 - DNA, 22 bp, Artificial
  AttB20 recombination site
caactttttggtacaaagttgt

SEQ ID NO: 36 - DNA, 27 bp, Artificial
  AttP20 recombination site
gttcaactttttggtacaaagttggca

SEQ ID NO: 37 - DNA, 24 bp, Artificial
  AttL20 recombination site
caactttttggtacaaagttggca

SEQ ID NO: 38 - DNA, 25 bp, Artificial
  AttR20 recombination site
gttcaactttttggtacaaagttgt

SEQ ID NO: 39 - DNA, 22 bp, Artificial
  AttB21 recombination site
caactttttaatacaaagttgt

SEQ ID NO: 40 - DNA, 27 bp, Artificial
  AttP21 recombination site
gttcaactttttaatacaaagttggca

SEQ ID NO: 41 - DNA, 24 bp, Artificial
  AttL21 recombination site
caactttttaatacaaagttggca

SEQ ID NO: 42 - DNA, 25 bp, Artificial
  AttR21 recombination site
gttcaactttttaatacaaagttgt

SEQ ID NO: 43 - DNA, 21 bp, Artificial
  att system core integrase binding site
caactttnnnnnnnaaagttg

SEQ ID NO: 44 - DNA, 21 bp, Artificial
  altered attB recombination site
caactttnnnnnnnaaacaag

SEQ ID NO: 45 - DNA, 55 bp, Artificial
  L140 primer
ggggacaactttgtacaaaaaagttgatgaccatgattacggattcactggccgt

SEQ ID NO: 46 - DNA, 58 bp, Artificial
  L340 primer
ggggacaactttgtataataaagttcatcacccgggctacagtgatgatggtggtgat

SEQ ID NO: 47 - DNA, 56 bp, Artificial
  R360 primer
ggggacaactttattatacaaagtttgatgaccatgattacggattcactggccgt

SEQ ID NO: 48 - DNA, 56 bp, Artificial
  R460 primer
ggggacaacttttctatacaaagttcatcacccgggctacagtgatgatggtggtg

SEQ ID NO: 49 - DNA, 56 bp, Artificial
  L4100 primer
ggggacaactttgtatagaaaagtttgatgaccatgattacggattcactggccgt

SEQ ID NO: 50 - DNA, 56 bp, Artificial
  L2100 primer
ggggacaactttgtacaagaaagttctcaatcacccgggctgcagtgatgatggtg

SEQ ID NO: 51 - DNA, 540 bp, Artificial
  20 kDa nucleotide sequence of ORF
atgaccatgattacggattcactggccgtcgttttacaacgtcgtgactggtacaaccctggcgttacccaacttaatcaccccgtggacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggtattccagctgagcggcgcaatcgagggtagggtcgcgaccatggaattcgatgcgtctgaattaacagatgcctcgacaacttacaaacttgttattaatggtaaaacattgaaaggcggaacaactactgaagctgttgatgctgctactgcagaaaaagtcttcaaacaatacgctaacgacaacggtgttgacggtgaatggacttacgacgatgcgactaagatctttacagttactgaaaaactgatcgaattccatcaccaccatcatcactgtagcccgggtgattga

SEQ ID NO: 52 - DNA, 792 bp, Artificial
  30 kDa nucleotide sequence of ORF
atgaccatgattacggattcactggccgtcgttttacaacgtcgtgactggtacaaccctggcgttacccaacttaatcaccccgtggacttccagttcaacatcagccgctacagtcaacagcaactgatggaaaccagccatcgccatctgctgcacgcggaagaaggcacatggctgaatatcgacggtttccatacggccatgggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagatgtcatgggtggcgacgactcctggagcccgtcagtatcggcggtattccagctgagcgccggtcgctaccattaccagttggtctggtgtcaacgtggaacagatgccgtgacaacttacaaacttgttattaatggtaaaacattgaaaggcgaaacaactactgaagctgttgatgctgctactgcagaaaaagtcttcaaacaatacgctaacgacaacggtgttgacggtgaatggacttacgacgatgcgactaagacctttacagttactgaaaaagacatcgaattccatcaccaccatcatcactgcagcccgggtgattga

SEQ ID NO: 53 - DNA, 1074 bp, Artificial
  40 kDa nucleotide sequence of ORF
atgaccatgattacggattcactggccgtcgttttacaacgtcgtgactggtacaaccctggcgttacccaacttaatcaccccgtggacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggtattccagctgagcggcgcaatcgagggtagggtcgcgaccatggttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcccatggaattcgatgcgtctgaattaacagatgcctcgacaacttacaaacttgttattaatggtaaaacattgaaaggcggaacaactactgaagctgttgatgctgctactgcagaaaaagtcttcaaacaatacgctaacgacaacggtgttgacggtgaatggacttacgacgatgcgactaagatctttacagttactgaaaaactgatcgaattccatcaccaccatcatcactgtagcccgggtgattga

SEQ ID NO: 54 - DNA, 1326 bp, Artificial
  50 kDa nucleotide sequence of ORF
atgaccatgattacggattcactggccgtcgttttacaacgtcgtgactggtacaaccctggcgttacccaacttaatcaccccgtggacttccagttcaacatcagccgctacagtcaacagcaactgatggaaaccagccatcgccatctgctgcacgcggaagaaggcacatggctgaatatcgacggtttccatacggccatggttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcccatgggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagatgtcatgggtggcgacgactcctggagcccgtcagtatcggcggtattccagctgagcgccggtcgctaccattaccagttggtctggtgtcaacgtggaacagatgccgtgacaacttacaaacttgttattaatggtaaaacattgaaaggcgaaacaactactgaagctgttgatgctgctactgcagaaaaagtcttcaaacaatacgctaacgacaacggtgttgacggtgaatggacttacgacgatgcgactaagacctttacagttactgaaaaagacatcgaattccatcaccaccatcatcactgcagcccgggtgattga

SEQ ID NO: 55 - DNA, 1593 bp, Artificial
  60 kDa nucleotide sequence of ORF
atgaccatgattacggattcactggccgtcgttttacaacgtcgtgactggtacaaccctggcgttacccaacttaatcaccccgtggacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggtattccagctgagcggcgcaatcgagggtagggtcgcgaccatggagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgaccatggaattcgatgcgtctgaattaacagatgcctcgacaacttacaaacttgttattaatggtaaaacattgaaaggcggaacaactactgaagctgttgatgctgctactgcagaaaaagtcttcaaacaatacgctaacgacaacggtgttgacggtgaatggacttacgacgatgcgactaagatctttacagttactgaaaaactgatcgaattccatcaccaccatcatcactgtagcccgggtgattga

SEQ ID NO: 56 - DNA, 1845 bp, Artificial
  70 kDa nucleotide sequence of ORF
atgaccatgattacggattcactggccgtcgttttacaacgtcgtgactggtacaaccctggcgttacccaacttaatcaccccgtggacttccagttcaacatcagccgctacagtcaacagcaactgatggaaaccagccatcgccatctgctgcacgcggaagaaggcacatggctgaatatcgacggtttccatacggccatggagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgaccatgggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagatgtcatgggtggcgacgactcctggagcccgtcagtatcggcggtattccagctgagcgccggtcgctaccattaccagttggtctggtgtcaacgtggaacagatgccgtgacaacttacaaacttgttattaatggtaaaacattgaaaggcgaaacaactactgaagctgttgatgctgctactgcagaaaaagtcttcaaacaatacgctaacgacaacggtgttgacggtgaatggacttacgacgatgcgactaagacctttacagttactgaaaaagacatcgaattccatcaccaccatcatcactgcagcccgggtgattga

SEQ ID NO: 57 - DNA, 2124 bp, Artificial
  80 kDa nucleotide sequence of ORF
atgaccatgattacggattcactggccgtcgttttacaacgtcgtgactggtacaaccctggcgttacccaacttaatcaccccgtggacttccagttcaacatcagccgctacagtcaacagcaactgatggaaaccagccatcgccatctgctgcacgcggaagaaggcacatggctgaatatcgacggtttccatacggccatggagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccggccccatgggtggcgacgactcctggagcccgtcagtatcggcggtattccagctgagcgccggtcgctaccattaccagttggtctggtgtcaacgtggaacagatgccgtgacaacttacaaacttgttattaatggtaaaacattgaaaggcgaaacaactactgaagctgttgatgctgctactgcagaaaaagtcttcaaacaatacgctaacgacaacggtgttgacggtgaatggacttacgacgatgcgactaagacctttacagttactgaaaaagacatcgaattccatcaccaccatcatcactgcagcccgggtgattga

SEQ ID NO: 58 - DNA, 2658 bp, Artificial
  100 kDa nucleotide sequence of ORF
atgaccatgattacggattcactggccgtcgttttacaacgtcgtgactggtacaaccctggcgttacccaacttaatcaccccgtggacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggtattccagctgagcggcgcaatcgagggtagggtcgcgaccatgggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtccatccatggaattcgatgcgtctgaattaacagatgcctcgacaacttacaaacttgttattaatggtaaaacattgaaaggcggaacaactactgaagctgttgatgctgctactgcagaaaaagtcttcaaacaatacgctaacgacaacggtgttgacggtgaatggacttacgacgatgcgactaagatctttacagttactgaaaaactgatcgaattccatcaccaccatcatcactgtagcccgggtgattga

SEQ ID NO: 59 - DNA, 3198 bp, Artificial
  120 kDa nucleotide sequence of ORF
atgaccatgattacggattcactggccgtcgttttacaacgtcgtgactggtacaaccctggcgttacccaacttaatcaccccgtggacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggtattccagctgagcggcgcaatcgagggtagggtcgcgaccatgggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggtattccagctgagcgccggtcgctaccattaccagttggtctggtgtcaaaaaggggatcctggtcctgttggtcctgttggtcctgctggtgcttttggcccaagaggtgccgccatggaattcgatgcgtctgaattaacagatgcctcgacaacttacaaacttgttattaatggtaaaacattgaaaggcggaacaactactgaagctgttgatgctgctactgcagaaaaagtcttcaaacaatacgctaacgacaacggtgttgacggtgaatggacttacgacgatgcgactaagatctttacagttactgaaaaactgatcgaattccatcaccaccatcatcactgtagcccgggtgattga

SEQ ID NO: 60 - protein, 357 aa, Artificial
  40 kDa protein fragment
MTMITDSLAVVLQRRDWYNPGVTQLNHPVDFQFNISRYSQQQLMETSHRHLLHAEEGTWLNIDGFHMGIGGDDSWSPSVSAVFQLSGAIEGRVATMVLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIPMEFDASELTDASTTYKLVINGKTLKGGTTTEAVDAATAEKVFKQYANDNGVDGEWTYDDATKIFTVTEKLIEFHHHHHHCSPGD

SEQ ID NO: 61 - protein, 530 aa, Artificial
  60 kDa protein fragment
MTMITDSLAVVLQRRDWYNPGVTQLNHPVDFQFNISRYSQQQLMETSHRHLLHAEEGTWLNIDGFHMGIGGDDSWSPSVSAVFQLSGAIEGRVATMEQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTMEFDASELTDASTTYKLVINGKTLKGGTTTEAVDAATAEKVFKQYANDNGVDGEWTYDDATKIFTVTEKLIEFHHHHHHCSPGD

SEQ ID NO: 62 - protein, 885 aa, Artificial
  100 kDa protein fragment
MTMITDSLAVVLQRRDWYNPGVTQLNHPVDFQFNISRYSQQQLMETSHRHLLHAEEGTWLNIDGFHMGIGGDDSWSPSVSAVFQLSGAIEGRVATMGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGPSMEFDASELTDASTTYKLVINGKTLKGGTTTEAVDAATAEKVFKQYANDNGVDGEWTYDDATKIFTVTEKLIEFHHHHHHCSPGD

(19) United States
(12) Patent Application Publication
(10) Pub. No.: US 2004/0253620 A1
(43) Pub. Date: Dec. 16, 2004

(54) Use of site specific recombination to prepare molecular markers

(75) Inventors: Louis Leong, Mission Viejo, CA (US); Joseph William Amshey, Encinitas, CA (US); Noel Janney, San Diego, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation, Carlsbad, CA (US)

(21) Appl. No.: 10/835,275

(22) Filed: Apr. 30, 2004

Related U.S. Application Data
(60) Provisional application No. 60/466,420, filed on Apr. 30, 2003.

(51) Int. Cl. C12Q 1/68

(52) U.S. Cl. 435/6

(57) ABSTRACT

The present invention relates to the fields of biotechnology and molecular biology. In particular, the present invention relates to methods for preparing marker molecules for identifying physical properties of molecular species separated by electrophoretic systems. The methods comprise joining multiple nucleic acid segments containing at least one recombination site under conditions favoring recombination. The present invention relates to both nucleic acid marker molecules and protein marker molecules, compositions comprising said marker molecules, and methods for preparing said marker molecules.

### CROSS REFERENCE TO RELATED APPLICATIONS

This application claims priority to U.S. Provisional Application No. 60/466,420, filed Apr. 30, 2003, which is entirely incorporated herein by reference.

### BACKGROUND OF THE INVENTION

1. Field of the Invention

The present invention relates to the fields of biotechnology and molecular biology. In particular, the present invention relates to methods for preparing marker molecules for identifying one or more physical properties of molecular species separated by virtue of a physical property of the species. The methods comprise joining multiple nucleic acid molecules containing at least one recombination site under conditions favoring recombination. The present invention relates to both nucleic acid marker molecules and protein marker molecules, compositions comprising said marker molecules, methods for preparing said marker molecules, kits comprising said marker molecules, and business methods for the production and distribution of said marker molecules and compositions.

2. Related Art

Gel Electrophoresis

Gel electrophoresis is a common procedure for the separation of biological molecules, such as deoxyribonucleic acid (DNA), ribonucleic acid (RNA), polypeptides and proteins. A common method of electrophoresis of proteins involves equilibrating the sample with a negatively-charged surfactant such as sodium dodecylsulfate (SDS) before electrophoresis. This causes proteins to have a net negative charge and thus migrate toward the anode. Nucleic acids are inherently charged by virtue of the phosphate groups linking the nucleosides. In gel electrophoresis, molecules are separated according to the rates at which an electric field causes them to migrate through a medium.

A commonly used variant of this technique consists of an aqueous gel enclosed in a glass tube or sandwiched as a slab between glass or plastic plates. The gel has an open molecular network structure, defining pores that are saturated with an electrically conductive buffered solution of a salt. These pores through the gel are large enough to admit passage of the migrating macromolecules. The gel is placed in a chamber in contact with buffer solutions which make electrical contact between the gel and the cathode or anode of an electrical power supply. Samples containing the macromolecules and a tracking dye are introduced into the gel. An electric potential is applied to the gel causing the sample macromolecules and tracking dye to migrate toward one of the electrodes depending on the charge on the macromolecule. The electrophoresis is halted just before the tracking dye reaches the end of the gel. The locations of the bands of separated macromolecules are then determined. By comparing the distance moved by particular bands in comparison to the tracking dye and macromolecules of known mobility, the mobility of other macromolecules can be determined. The size of the macromolecule can then be calculated.

For nucleic acids, gel electrophoresis involves separation of species on the basis of size (length or molecular weight), and conformation (linear vs. nicked circles vs. covalently closed circles vs. single stranded vs. double stranded). For a given conformation, electrophoretic mobility is inversely related to size.

Conventional agarose gel electrophoresis is commonly used for the separation of nucleic acid fragments within a practical resolution limit of 50 kilobasepairs (kbp) (Cantor, C. R. and Schimmel, P. R. (1980) Biophysical Chemistry, Vol. III, pp. 1012-1036, Freeman, San Francisco; and Maniatis, T. et al. (1982) Molecular Cloning: A Laboratory Manual, Cold Spring Harbor Laboratory Press, Cold Spring Harbor, N.Y.). A method called pulsed field gel electrophoresis (PFGE) has been developed to provide separation of DNA molecules up to 2 megabasepairs (Mbp) (Schwartz, D. C. et al. (1983) Cold Spring Harbor Symp. Quant. Biol. 47:189-195.; and Schwartz, D. C. and Cantor, C. R. (1984) Cell 37:67-75).

A number of mixtures of nucleic acid fragments of known molecular weight (“ladders”) are commercially available that can be used as markers for determining or estimating the sizes of nucleic acid molecules during gel electrophoresis. One type of ladder is constructed by digesting plasmids or bacteriophage with one or more restriction enzymes. The size of the marker fragments will depend upon the natural location of the restriction enzyme site within the molecule to be digested and will produce a quasi-random size distribution. For example digestion of bacteriophage 8 (lambda) with HindIII produces fragments of 23130, 9416, 6557, 4361, 2322, 2027, 564, and 125 base pairs (bp) (See, e.g., Cat. No. 15612013, Invitrogen Corporation, Carlsbad, Calif. (online catalogue at www.invitrogen.com)).

Alternatively, a ladder may comprise fragments which vary linearly with molecular weight, i.e., adjacent bands may differ by about 1000 base pairs (e.g. 1 Kb DNA Ladder, Cat. No. 15615024, Invitrogen Corporation, Carlsbad, Calif.), 100 base pairs (e.g. 100 bp DNA Ladder, Cat. No. 15628019, Invitrogen Corporation, Carlsbad, Calif.), or 123 bp (e.g. 123 bp DNA Ladder, Cat. No. 15613029, Invitrogen Corporation, Carlsbad, Calif.).

Nucleic acids are generally visualized in agarose gels following electrophoresis by staining with the fluorescent dye ethidium bromide (Sharp et al. (1973) Biochemistry 12:3055). Ethidium bromide contains a planar group that intercalates between nucleic acid bases. The fixed position of its planar groups and its close proximity to the nucleic acid bases cause the ethidium bromide bound to the nucleic acid to display an increased fluorescent yield compared to that of ethidium bromide in free solution (See Sambrook et al. (1989) Molecular Cloning, 2^{nd} edition, Cold Spring Harbor Laboratory Press, Cold Spring Harbor, N.Y., page 6.15). The molecular mass of a nucleic acid fragment can be determined following agarose gel electrophoresis and ethidium bromide staining by comparing the intensity of the fluorescence of a fragment of unknown molecular mass with the intensity of a similarly sized fragment of known molecular mass. Other dyes such as e.g., CYBR Green are also used in the art.

For proteins, the determination of molecular weight and isoelectric point are of importance. The analysis may be done through the determination of mobility of the protein under chromatographic or electrophoretic conditions. Because these methods do not yield absolute values and exhibit large variation, it is necessary to compare properties to one or more standard proteins whose characteristics (e.g., electromobility) are known.

A common method to determine the characteristics of protein in a mixture of proteins is the Western Blot. In this method, a mixture of proteins is separated by polyacrylamide gel electrophoresis (PAGE). After an optional stain, the gel is placed onto a protein binding membrane such as nitrocellulose or polyvinylidene fluoride (PVDF) and electroeluted in a blotting chamber, which causes the protein to migrate from the gel to the membrane. Remaining binding sites on the membrane are saturated with a blocking reagent such as albumin.

Commonly, the detection of the protein to be analyzed is accomplished through incubation with a primary antibody that binds with the highest possible affinity and specificity to the protein. A second step involves incubation with a so-called secondary antibody. This antibody is specific for the Fc portion of the primary antibody and is typically conjugated with a marker, commonly in the form of an enzyme, fluorescent or chemiluminescent molecule. In this case, the enzyme is able to further convert an added substrate to a chromogenic insoluble product which allows for detection of the position of the respective protein or liberate a chemiluminescent compound upon cleavage of the substrate.

Isoelectric focusing (IEF) is an electrophoresis method based on the migration of a molecular species in a pH gradient to its isoelectric point (pI). The pH gradient is established by subjecting an ampholyte solution containing a large number of different-pI species to an electric field, usually in a cross-linked matrix such as a gel. Analytes added to the ampholyte-containing medium will migrate to their isoelectric points along the pH gradient when an electrical potential difference is applied across the gel.

For complex samples, multidimensional electrophoresis methods have been employed to better separate species that co-migrate when only a single electrophoresis dimension is used. Common among these is two dimensional electrophoresis or 2D-E. For 2D-E analysis of proteins, for example, the sample is usually fractionated first by IEF in a tube or strip gel to exploit the dependence of each protein's net charge on pH. Next, the gel containing the proteins separated by pI is extruded from the tube in the case of a tube gel, equilibrated with SDS and laid horizontally along one edge of a slab gel, typically a cross-linked polyacrylamide gel containing SDS. Other methods for IEF fractionation allow pieces or strips of gel supported on non-conductive backing to be laid directly onto the slab of gel. Electrophoresis is then performed in the second dimension, perpendicular to the first, and the proteins separate on the basis of molecular weight. This process is referred to as SDS polyacrylamide gel electrophoresis or SDS-PAGE. The rate of migration of macromolecules through the SDS-PAGE gel depends upon four principle factors: the porosity of the gel; the size and shape of the macromolecule; the field strength; and the charge density of the macromolecule. These four factors should be precisely controlled and reproducible from gel to gel and from sample to sample. However, maintaining uniformity between gels is difficult because each of these factors is sensitive to many variables in the chemistry of the gel and the other reagents in the system as well as the characteristics of the macromolecules. Thus, proteins having similar net charges, which are not separated well in the first dimension (IEF), will separate according to variations of the other principle factors in the second dimension (SDS-PAGE). Since these two separation methods depend on independent properties, the overall resolution is approximately the product of the resolution in each dimension.

Essential to the practice of many of these electrophoretic techniques, including 2D-E and SDS-PAGE, are molecular marker standards, i.e. standard molecules with known sizes (e.g. molecular weights or length) and pIs. Molecular markers are used as benchmarks in electrophoresis systems for comparison of physical properties with the unknown samples of interest. Although there are numerous applications for molecular markers, some particular examples include: conventional two-dimensional gel electrophoresis using broad pH range immobilized pH gradient (IPG) strips, overlapping two-dimensional gel electrophoresis using narrow pH range IPG strips, stand-alone SDS-PAGE, IEF gels with carrier ampholytes, capillary electrophoresis, electrokinetic chromatography. Many other forms of gel electrophoresis are well known to those of skill in the art.

Thus, it is desirable to have reliable standard markers with well-defined properties with which to compare an unknown sample.

Site-Specific Recombinases

Site-specific recombinases are proteins that are present in many organisms (e.g., yeast, viruses and bacteria) and have been characterized as having both endonuclease and ligase properties. These recombinases (along with associated proteins in some cases) recognize specific sequences of bases in a nucleic acid molecule and exchange the nucleic acid segments flanking those sequences. The recombinases and associated proteins are collectively referred to as “recombination proteins” (see, e.g., Landy, A., Current Opinion in Biotechnology 3:699-707 (1993)).

Numerous recombination systems from various organisms have been described. See, e.g., Hoess, et al., Nucleic Acids Research 14(6):2287 (1986); Abremski, et al., J. Biol. Chem. 261(1):391 (1986); Campbell, J. Bacteriol. 174(23):7495 (1992); Qian, et al., J. Biol. Chem. 267(11):7794 (1992); Araki, et al., J. Mol. Biol. 225(1):25 (1992); Maeser and Kahnmann, Mol. Gen. Genet. 230:170-176) (1991); Esposito, et al., Nucl. Acids Res. 25(18):3605 (1997). Many of these belong to the integrase family of recombinases (Argos, et al., EMBO J. 5:433-440 (1986); Voziyanov, et al., Nucl. Acids Res. 27:930 (1999)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2 Φ circle plasmid (Broach, et al., Cell 29:227-234 (1982)).

Transposons

Transposons are mobile genetic elements. Transposons are structurally variable, but typically encode a transposition catalyzing enzyme, termed a transposase, flanked by DNA sequences organized in inverted orientations. For a more thorough discussion of the characteristics of transposons, one may consult Mobile Genetic Elements, D. J. Sherratt, Ed., Oxford University Press (1995) and Mobile DNA, D. E. Berg and M. M. Howe, Eds., American Society for Microbiology (1989), Washington, D.C. both of which are specifically incorporated herein by reference.

Transposons have been used to insert DNA into target DNA. As a general rule, the insertion of transposons into target DNA is a random event. One exception to this rule is the insertion of transposon Tn7. Transposon Tn7 can integrate itself into a specific site in the E. coli genome as one part of its life cycle (Stellwagen, A. E., and Craig, N. L. Trends in Biochemical Sciences 23, 486-490, 1998 specifically incorporated herein by reference). This site specific insertion has been used in vivo to manipulate the baculovirus genome (Lucklow et al., J. Virol. 67:4566-4579 (1993) specifically incorporated herein by reference). The site specificity of Tn7 is atypical of transposable elements whose hallmark is movement to random positions in acceptor DNA molecules. For the purposes of this application, transposition will be used to refer to random or quasi-random movement, unless otherwise specified, whereas recombination will be used to refer to site specific recombination events. Thus, the site specific insertion of Tn7 into the attTn 7 site would be referred to as a recombination event while the random insertion of Tn3 or Tn5 would be referred to as a transposition event.

York, et al. (Nucleic Acids Research, 26(8):1927-1933, (1998)) disclose an in vitro method for the generation of nested deletions based upon an intramolecular transposition within a plasmid using Tn5. A vector containing a kanamycin resistance gene flanked by two 19 base pair Tn5 transposase recognition sequences and a target DNA sequence was incubated in vitro in the presence of purified transposase protein. Under the conditions of low DNA concentration employed, the intramolecular transposition reaction was favored and was successfully used to generate a set of nested deletions in the target DNA. The authors suggested that this system might be used to generate C-terminal truncations in a protein encoded by the target DNA by the inclusion of stop signals in all three reading frames adjacent to the recognition sequences. In addition, the authors suggested that the inclusion of a His tag and kinase region might be used to generate N-terminal deletion proteins for further analysis.

Devine, et al., (Nucleic Acids Research, 22:3765-3772 (1994) and U.S. Pat. Nos. 5,677,170 and 5,843,772, all of which are specifically incorporated herein by reference) disclose the construction of artificial transposons for the insertion of DNA segments into recipient DNA molecules in vitro. The system makes use of the insertion-catalyzing enzyme of yeast TY1 virus-like particles as a source of transposase activity. The DNA segment of interest is cloned, using standard methods, between the ends of the transposon-like element TY1. In the presence of the TY1 insertion-catalyzing enzyme, the resulting element integrates randomly into a second target DNA molecule.

Another class of mobile genetic elements are integrons. Integrons generally consist of a 5′- and a 3′-conserved sequence flanking a variable sequence. Typically, the 5′-conserved sequence contains the coding information for an integrase protein. The integrase protein may catalyze site-specific recombination at a variety of recombination sites including attI, attC as well as other types of sites (see Francia et al., J. Bacteriology 181(21):6844-6849, 1999, and references cited therein).

Recombination Sites

Whether the reactions discussed above are termed recombination, transposition or integration and are catalyzed by a recombinase or integrase, they share the key feature of specific recognition sequences, often termed “recombination sites,” on the nucleic acid molecules participating in the reactions. These recombination sites are sections or segments of nucleic acid on the participating nucleic acid molecules that are recognized and bound by the recombination proteins during the initial stages of integration or recombination. For example, the recombination site for Cre recombinase is loxP which is a 34 base pair sequence comprised of two 13 base pair inverted repeats (serving as the recombinase binding sites) flanking an 8 base pair core sequence. (See FIG. 1 of Sauer, B., Curr. Opin. Biotech. 5:521-527 (1994).) Other examples of recognition sequences include the attB, attP, attL, and attR sequences which are recognized by the recombination protein λ Int. attB is an approximately 25 base pair sequence containing two 9 base pair core-type Int binding sites and a 7 base pair overlap region, while attP is an approximately 240 base pair sequence containing core-type Int binding sites and arm-type Int binding sites as well as sites for auxiliary proteins integration host factor (IHF), FIS and excisionase (Xis). (See Landy, Curr. Opin. Biotech. 3:699-707 (1993).)

Stop Codons and Suppressor tRNAs

Three codons are used by both eukaryotes and prokaryotes to signal the end of gene. When transcribed into mRNA, these codons have the following sequences: UAG (amber), UGA (opal) and UAA (ochre). Under most circumstances, the cell does not contain any tRNA molecules that recognize these codons. Thus, when a ribosome translating an mRNA reaches one of these codons, the ribosome stalls and falls of the RNA, terminating translation of the mRNA. The release of the ribosome from the mRNA is mediated by specific factors (see S. Mottagui-Tabar, NAR 26(11), 2789, 1998). A gene with an in-frame stop codon (TAA, TAG, or TGA) will ordinarily encode a protein with a native carboxy terminus. However, suppressor tRNAs, can result in the insertion of amino acids and continuation of translation past stop codons.

Mutant tRNA molecules that recognize what are ordinarily stop codons suppress the termination of translation of an mRNA molecule and are termed suppressor tRNAs. A number of such suppressor tRNAs have been found. Examples include, but are not limited to, the supE, supP, supD, supF and supZ suppressors which suppress the termination of translation of the amber stop codon, supB, glT, supL, supN, supC and supM suppressors which suppress the function of the ochre stop codon and glyT, trpT and Su-9 which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. For a more detailed discussion of suppressor tRNAs, see Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.

Mutations which enhance the efficiency of translation terminators or termination suppressors, i.e., increase the read-through of the stop codon, have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rp/L gene.

Under ordinary circumstances, host cells would not be expected to be healthy if suppression of stop codons is too efficient. This is because of the thousands or tens of thousands of genes in a genome, a substantial fraction will naturally have one of the three stop codons; complete read-through of these would result in a large number of aberrant proteins containing additional amino acids at their carboxy termini. If some level of suppressing tRNA is present, there is a race between the incorporation of the amino acid and the release of the ribosome. Higher levels of tRNA may lead to more read-through although other factors, such as the codon context, can influence the efficiency of suppression.

Organisms ordinarily have multiple genes for tRNAs. Combined with the redundancy of the genetic code (multiple codons for many of the amino acids), mutation of one tRNA gene to a suppressor tRNA status does not lead to high levels of suppression. The TAA stop codon is the strongest, and most difficult to suppress. The TGA is the weakest, and naturally (in E. coli) has a read-through rate of 3%. The TAG (amber) codon is relatively tight, with a read-through of ˜1% without suppression. In addition, the amber codon can be suppressed with efficiencies on the order of 50% with naturally occurring suppressor mutants.

Suppression is known in bacteria, bacteriophages, yeast, flies, plants and other eukaryotic cells including mammalian cells. For example, Capone, et al. (Molecular and Cellular Biology 6(9):3059-3067, 1986) demonstrated that suppressor tRNAs derived from mammalian tRNAs could be used to suppress a stop codon in mammalian cells. A copy of the E. coli chloramphenicol acetyltransferase (cat) gene having a stop codon in place of the codon for serine 27 was transfected into mammalian cells along with a gene encoding a human serine tRNA which had been mutated to form an amber, ochre, or opal suppressor derivative of the gene. Successful expression of the cat gene was observed. An inducible mammalian amber suppressor has been used to suppress a mutation in the replicase gene of polio virus and cell lines expressing the suppressor were successfully used to propagate the mutated virus (Sedivy, et al., (1987) Cell 50: 379-389). The context effects on the efficiency of suppression of stop codons by suppressor tRNAs has been shown to be different in mammalian cells as compared to E. coli (Phillips-Jones, et al., (1995) Molecular and Cellular Biology 15(12): 6593-6600, Martin, et al., (1993) Biochemical Society Transactions 21(4):846-51) Since some human diseases are caused by nonsense mutations in essential genes, the potential of suppression for gene therapy has long been recognized (see Temple, et al. (1982) Nature 296(5857):537-40). The suppression of single and double nonsense mutations introduced into the diphtheria toxin A-gene has been used as the basis of a binary system for toxin gene therapy (Robinson, et al., (1995) Human Gene Therapy 6:137-143).

Conventional Nucleic Acid Cloning

The cloning of nucleic acid segments currently occurs as a daily routine in many research labs and as a prerequisite step in many genetic analyses. The purpose of these clonings is various, however, two general purposes can be considered: (1) the initial cloning of nucleic acid from large DNA or RNA segments (chromosomes, YACs, PCR fragments, mRNA, etc.), done in a relative handful of known vectors such as pUC, pGem, pBlueScript, and (2) the subcloning of these nucleic acid segments into specialized vectors for functional analysis. A great deal of time and effort is expended both in the transfer of nucleic acid segments from the initial cloning vectors to the more specialized vectors. This transfer is called subcloning.

The basic methods for cloning have been known for many years and have changed little during that time. A typical cloning protocol is as follows:

(1) digest the nucleic acid of interest with one or two restriction enzymes;

(2) gel purify the nucleic acid segment of interest when known;

(3) prepare the vector by cutting with appropriate restriction enzymes, treating with alkaline phosphatase, gel purify etc., as appropriate;

(4) ligate the nucleic acid segment to the vector, with appropriate controls to eliminate background of uncut and self-ligated vector;

(5) introduce the resulting vector into an E. coli host cell;

(6) pick selected colonies and grow small cultures overnight;

(7) make nucleic acid minipreps; and

(8) analyze the isolated plasmid on agarose gels (often after diagnostic restriction enzyme digestion) or by PCR.

The specialized vectors used for subcloning nucleic acid segments are functionally diverse. These include but are not limited to: vectors for expressing nucleic acid molecules in various organisms; for regulating nucleic acid molecule expression; for providing tags to aid in protein purification or to allow tracking of proteins in cells; for modifying the cloned nucleic acid segment (e.g., generating deletions); for the synthesis of probes (e.g., riboprobes); for the preparation of templates for nucleic acid sequencing; for the identification of protein coding regions; for the fusion of various protein-coding regions; to provide large amounts of the nucleic acid of interest, etc. It is common that a particular investigation will involve subcloning the nucleic acid segment of interest into several different specialized vectors.

As known in the art, simple subclonings can be done in one day (e.g., the nucleic acid segment is not large and the restriction sites are compatible with those of the subcloning vector). However, many other subclonings can take several weeks, especially those involving unknown sequences, long fragments, toxic genes, unsuitable placement of restriction sites, high backgrounds, impure enzymes, etc. One of the most tedious and time consuming type of subcloning involves the sequential addition of several nucleic acid segments to a vector in order to construct a desired clone. One example of this type of cloning is in the construction of gene targeting vectors. Gene targeting vectors typically include two nucleic acid segments, each identical to a portion of the target gene, flanking a selectable marker. In order to construct such a vector, it may be necessary to clone each segment sequentially, i.e., first one gene fragment is inserted into the vector, then the selectable marker and then the second fragment of the target gene. This may require a number of digestion, purification, ligation and isolation steps for each fragment cloned. Subcloning nucleic acid fragments is thus often viewed as a chore to be done as few times as possible.

Several methods for facilitating the cloning of nucleic acid segments have been described, e.g., as in the following references.

Ferguson, J., et al., Gene 16:191 (1981), disclose a family of vectors for subcloning fragments of yeast nucleic acids. The vectors encode kanamycin resistance. Clones of longer yeast nucleic acid segments can be partially digested and ligated into the subcloning vectors. If the original cloning vector conveys resistance to ampicillin, no purification is necessary prior to transformation, since the selection will be for kanamycin.

Hashimoto-Gotoh, T., et al., Gene 41:125 (1986), disclose a subcloning vector with unique cloning sites within a streptomycin sensitivity gene; in a streptomycin-resistant host, only plasmids with inserts or deletions in the dominant sensitivity gene will survive streptomycin selection.

Traditional subclonings using restriction and ligase enzymes are time consuming and relatively unreliable. Considerable labor is expended, and if two or more days later the desired subclone can not be found among the candidate plasmids, the entire process must then be repeated with alternative conditions attempted.

Recombinational Cloning

Cloning systems that utilize recombination at defined recombination sites have been previously described in the related applications listed above, and in U.S. application Ser. No. 09/177,387, filed Oct. 23, 1998; U.S. application Ser. No. 09/517,466, filed Mar. 2, 2000; and U.S. Pat. Nos. 5,888,732 and 6,143,557, all of which are specifically incorporated herein by reference. In brief, the GATEWAYJ Cloning System utilizes vectors that contain at least one recombination site to clone desired nucleic acid molecules in vivo or in vitro. More specifically, the system utilizes vectors that contain at least two different site-specific recombination sites based on the bacteriophage lambda system (e.g., att1 and att2) that are mutated from the wild-type (att0) sites. Each mutated site has a unique specificity for its cognate partner att site (i.e., its binding partner recombination site) of the same type (for example, attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the GATEWAYJ system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

Mutating specific residues in the core region of the att site can generate a large number of different att sites. As with the att1 and att2 sites utilized in GATEWAYJ, each additional mutation potentially creates a novel att site with unique specificity that will recombine only with its cognate partner att site bearing the same mutation and will not cross-react with any other mutant or wild-type att site. Novel mutated att sites (e.g., attB 1-10, attP 1-10, attR 1-10 and attL 1-10) are described in previous patent application Ser. No. 09/517,466, filed Mar. 2, 2000, which is specifically incorporated herein by reference. Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites; loxP site mutants, variants or derivatives such as loxP511 (see U.S. Pat. No. 5,851,808); frt sites; frt site mutants, variants or derivatives; dif sites; dif site mutants, variants or derivatives; psi sites; psi site mutants, variants or derivatives; cer sites; and cer site mutants, variants or derivatives.

### BRIEF SUMMARY OF THE INVENTION

The present invention is generally directed to compositions of marker molecules useful for identifying one or more physical characteristics of molecular species. For proteins, physical characteristics include isoelectric point (pI) and/or size as determined by molecular weight; for nucleic acids, physical characteristics include size (length) as determined by the number of base pairs (bp). The invention is further directed to methods for preparing said marker molecules in a predictable, ordered and reproducible fashion. The methods involve, for example, using nucleic acid molecules as modular building blocks which may be joined to form an assortment of product nucleic acid molecules. The product nucleic acid molecules may themselves be used as marker molecules or may in turn be used as starting nucleic acid molecules (or in this case as intermediates) to prepare other product nucleic acid molecules. Alternatively, the product nucleic acid molecules may be used to generate RNAs and/or express proteins in vivo or in vitro which are suitable for use as marker molecules.

The marker molecules may be nucleic acid molecules (nucleic acid markers such as DNA or RNA molecules) or proteins (protein markers) and, when present with other marker molecules, generally separate to give identifiable bands or spots under conditions which separate molecular species according to one or more physical property of the species.

The present invention is also directed to compositions comprising these marker molecules and methods of using these marker molecules and/or compositions to identify one or more physical characteristics of one or more (e.g., 1, 2, 3, 4, 5, 6, 7, 8, 9, 10, 11, 12, 13, 14, 15, 20, etc.) molecular species. The invention further provides methods for making these marker molecules and/or compositions and kits comprising these marker molecules and/or compositions.

In one embodiment, the present invention relates to marker molecule compositions comprising a plurality of protein marker molecules. Optionally, two or more of the protein marker molecules may have the same pI and same molecular weight. In another embodiment, the present invention relates to marker molecule compositions comprising a plurality of protein marker molecules having the same pI and different molecular weights. In yet another embodiment, the present invention relates to marker molecule compositions comprising a plurality of protein marker molecules having different pIs and different molecular weights. In a further embodiment, the present invention relates to marker molecule compositions comprising a plurality of protein marker molecules having different pIs and the same molecular weight. The invention also relates to methods for producing and using these protein marker molecules and compositions, for example, as described elsewhere herein.

In another embodiment, the present invention relates to a protein marker molecule having a molecular weight ranging from about 5 kilodaltons (kDa) to about 500 kDa, from about 5 kDa to about 400 kDa, from about 5 kDa to about 250 kDa, from about 5 kDa to about 125 kDa, from about 5 kDa to about 100 kDa, from about 5 kDa to about 75 kDa, from about 5 kDa to about 50 kDa, from about 5 kDa to about 25 kDa, from about 10 kilodaltons (kDa) to about 500 kDa, from about 10 kDa to about 400 kDa, from about 10 kDa to about 250 kDa, from about 10 kDa to about 125 kDa, from about 10 kDa to about 100 kDa, from about 10 kDa to about 75 kDa, from about 10 kDa to about 50 kDa, from about 10 kDa to about 25 kDa, from about 5 kilodaltons (kDa) to about 500 kDa, from about 25 kDa to about 400 kDa, from about 25 kDa to about 250 kDa, from about 25 kDa to about 125 kDa, from about 25 kDa to about 100 kDa, from about 25 kDa to about 75 kDa, from about 25 kDa to about 50 kDa, or from about 25 kDa to about 35 kDa as well as compositions comprising one or more (e.g., 1, 2, 3, 4, 5, 6, 7, 8, 9, 10, 11, 12, 13, 14, 15, 20, etc.) such proteins marker molecules. The invention also relates to methods for producing and using these protein marker molecules and compositions, for example, as described elsewhere herein.

In an additional embodiment, the present invention relates to a protein marker molecule having a molecular weight ranging from about 5 kDa to about 10 kDa, from about 10 kDa to about 20 kDa, from about 20 kDa to about 30 kDa, from about 30 kDa to about 40 kDa, from about 40 kDa to about 50 kDa, from about 50 kDa to about 60 kDa, from about 60 kDa to about 80 kDa, from about 80 kDa to about 100 kDa, from about 100 kDa to about 120 kDa, from about 120 kDa to about 200 kDa, or from about 200 kDa to about 400 kDa, as well as compositions comprising one or more (e.g., 1, 2, 3, 4, 5, 6, 7, 8, 9, 10, 11, 12, 13, 14, 15, 20, etc.) such proteins marker molecules. The invention also relates to methods for producing and using these protein marker molecules and compositions, for example, as described elsewhere herein.

In a further embodiment, the present invention relates to a protein marker molecule having a molecular weight of about 20 kDa, about 30 kDa, about 40 kDa, about 50 kDa, about 60 kDa, about 80 kDa, about 100 kDa, about 120 kDa, or about 200 kDa as well as compositions comprising one or more (e.g., 1, 2, 3, 4, 5, 6, 7, 8, 9, 10, 11, 12, 13, 14, 15, 20, etc.) such proteins marker molecules. The invention also relates to methods for producing and using these protein marker molecules and compositions, for example, as described elsewhere herein.

In one embodiment, the protein marker molecule may have an isoelectric point (pI) from about 0 to about 14, from about 2 to about 12, from about 3 to about 11, from about 3 to about 13, from about 4 to about 10, from about 5 to about 9, or from about 6 to about 8.

In another embodiment, the protein marker molecule may have a pI from about 0 to about 12, from about 0 to about 10, from about 0 to about 8, from about 0 to about 6, from about 0 to about 4, from about 0 to about 2, from about 2 to about 14, from about 2 to about 12, from about 2 to about 8, from about 2 to about 6, from about 2 to about 4, from about 3 to about 13, from about 4 to about 14, from about 4 to about 12, from about 4 to about 10, from about 4 to about 8, or from about 4 to about 6.

In another embodiment, the protein marker molecule may have one or more domains or regions which facilitate visualization, isolation or confer other activities to the protein marker molecule, including but not limited to, enzymatic activity and/or the ability to bind other proteins of interest.

In a further embodiment, the present invention relates to marker molecule compositions comprising a collection of two or more (e.g., 2, 3, 4, 5, 6, 7, 8, 9, 10, 11, 12, 13, 14, 15, 20, etc.) marker molecules of the present invention wherein, for protein marker molecules, the compositions comprise marker molecules having an assortment of molecular weights and/or isoelectric points (pI and wherein for nucleic acid marker molecules, the compositions comprise marker molecules having an assortment of sizes (lengths) as determined by the number of bp. The invention also relates to methods for producing and using these protein marker molecules and compositions, for example, as described elsewhere herein.

In another embodiment, the present invention is directed to compositions comprising one or more (e.g., 1, 2, 3, 4, 5, 6, 7, 8, 9, 10, 11, 12, 13, 14, 15, 20, etc.) protein marker molecules. The compositions may comprise protein marker molecules having the same physical characteristics or protein marker molecules having an assortment of physical characteristics. In one embodiment, the compositions comprise protein marker molecules having an assortment of molecular weights. For example, in one embodiment, the compositions of the invention comprise a plurality of protein marker molecules having molecular weights of about 20 kDa, about 30 kDa, about 40 kDa, about 50 kDa, about 60 kDa, about 80 kDa, about 100 kDa, about 120 kDa, and about 200 kDa. The invention also relates to methods for producing and using these compositions, for example, as described elsewhere herein.

In a further embodiment, the compositions of the invention comprise a plurality of protein marker molecules, each having a molecular weight of 15 kDa and a pI of 4.0, a molecular weight of 15 kDa and a pI of 6.0, molecular weight of 20 kDa and a pI of 5.0, a molecular weight of 25 kDa and a pI of 4.5, a molecular weight of 60 kDa and a pI of 5.5, a molecular weight of 60 kDa and a pI of 3.0, or a molecular weight of 60 kDa and a pI of 6.5.

In another embodiment, the present invention relates to a nucleic acid marker molecule comprising about 50 to about 100 bp, about 100 to about 200 bp, about 200 to about 300 bp, about 300 to about 500 bp, about 500 to about 1000 bp, about 1000 to about 1500 bp, about 1500 to about 2000 bp, about 2000 to about 3000 bp, about 3000 to about 5000 bp, about 5000 to about 7500 bp, about 7500 to about 10,000 bp, about 10,000 to about 15,000 bp, about 15,000 to about 20,000 bp, about 20,000 to about 25,000 bp, about 25,000 to about 30,000 bp, about 30,000 to about 40,000 bp, about 40,000 to about 50,000 bp, about 50,000 to about 60,000 bp, or about 60,000 to about 75,000 bp. In yet another embodiment, the invention relates to a nucleic acid marker molecule comprising about 100 bp, about 200 bp, about 300 bp, about 400 bp, about 500 bp, about 600 bp, about 650 bp, about 850 bp, about 1000 bp, about 1650 bp, about 2000 bp, about 3000 bp, about 4000 bp, about 5000 bp, about 6000 bp, about 7000 bp, about 8000 bp, about 9000 bp, about 10,000 bp, about 11,000 bp, and/or about 12,000 bp as well as compositions comprising one or more (e.g., 1, 2, 3, 4, 5, 6, 7, 8, 9, 10, 11, 12, 13, 14, 15, 20, etc.) such nucleic acid marker molecules. The invention also relates to methods for producing and using these marker molecules and compositions, for example, as described elsewhere herein.

In another embodiment, the marker molecule has one or more regions or segments which facilitate visualization, isolation or confer other activities to the marker molecule, including but not limited to, enzymatic activity and/or the ability to bind proteins.

In an additional embodiment, the present invention relates to nucleic acid marker molecule compositions comprising about 100 bp to about 75,000 bp, about 100 bp to about 60,000 bp, about 100 bp to about 50,000 bp, about 100 bp to about 40,000 bp, about 100 bp to about 30,000 bp, about 100 bp to about 25,000 bp, about 100 bp to about 20,000 bp, from about 100 bp to about 19,000 bp, from about 100 bp to about 18,000 bp, from about 100 bp to about 17,000 bp, from about 100 bp to about 16,000 bp, from about 100 bp to about 15,000 bp, from about 100 bp to about 14,000 bp, from about 100 bp to about 13,000 bp, from about 100 bp to about 12,000 bp, from about 100 bp to about 11,000 bp, from about 100 bp to about 10,000 bp, from about 100 bp to about 9,000 bp, from about 100 bp to about 8,000 bp, from about 100 bp to about 7,000 bp, from about 100 bp to about 6,000 bp, from about 100 bp to about 5,000 bp, from about 100 bp to about 4,000 bp, from about 100 bp to about 3,000 bp, from about 100 bp to about 2,900 bp, from about 100 bp to about 2,800 bp, from about 100 bp to about 2,700 bp, from about 100 bp to about 2,600 bp, from about 100 bp to about 2,500 bp, from about 100 bp to about 2,400 bp, from about 100 bp to about 2,300 bp, from about 100 bp to about bp, from about 100 bp to about bp, from about 100 bp to about 2,200 bp, from about 100 bp to about 2,100 bp, from about 100 bp to about 2,000 bp, from about 100 bp to about 1,900 bp, from about 100 bp to about bp 1,800, from about 100 bp to about 1,700 bp, from about 100 bp to about 1,600 bp, from about 100 bp to about 1,500 bp, from about 100 bp to about 1,400 bp, from about 100 bp to about 1,300 bp, from about 100 bp to about 1,200 bp, from about 100 bp to about 1,100 bp, from about 100 bp to about 1,000 bp, from about 100 bp to about 900 bp, from about 100 bp to about 800 bp, from about 100 bp to about 700 bp, from about 100 bp to about 600 bp, from about 100 bp to about 500 bp, from about 100 bp to about 400 bp, from about 100 bp to about 300 bp, or from about 100 bp to about 200 bp as well as compositions comprising one or more (e.g., 1, 2, 3, 4, 5, 6, 7, 8, 9, 10, 11, 12, 13, 14, 15, 20, etc.) such nucleic acid marker molecules. The invention also relates to methods for producing and using these marker molecules and compositions, for example, as described elsewhere herein.

In another embodiment, the present invention relates to marker molecule compositions comprising nucleic acid marker molecules having an assortment of different sizes as determined by the number of bp. For example, in one embodiment, the compositions of the invention comprise a plurality of nucleic acid marker molecules having lengths of about 100 bp, about 200 bp, about 300 bp, about 400 bp, about 500 bp, about 600 bp, about 650 bp, about 850 bp, about 1000 bp, about 1650 bp, about 2000 bp, about 3000 bp, about 4000 bp, about 5000 bp, about 6000 bp, about 7000 bp, about 8000 bp, about 9000 bp, about 10,000 bp, about 11,000 bp, and about 12,000 bp as well as compositions comprising one or more (e.g., 1, 2, 3, 4, 5, 6, 7, 8, 9, 10, 11, 12, 13, 14, 15, 20, etc.) such nucleic acid marker molecules. The invention also relates to methods for producing and using these marker molecules and compositions, for example, as described elsewhere herein.

In another embodiment, the present invention relates to methods of separating two or more molecular species present in a sample using methods which separate molecules by virtue of one or more physical properties of the species, such as molecular weight, charge and/or molecular shape or conformation. Such methods are well known in the art and include, but are not limited to, gel electrophoresis, gel filtration, isoelectric focusing, fast performance liquid chromatography (FPLC), and high pressure liquid chromatography (HPLC). In one aspect, the methods comprise adding the marker molecule compositions of the invention to a sample containing one or more molecular species, and subjecting the sample to conditions which favor separation of the molecular species. In another aspect, the methods comprise adding the marker molecule compositions of the invention alongside a sample containing one or more molecular species, and subjecting the marker molecule compositions and the sample to conditions which favor separation of the molecular species.

In another aspect, the present invention relates to methods of separating two or more molecular species present in a sample by gel electrophoresis, comprising adding the marker molecule compositions of the present invention to the sample containing one or more molecular species, applying the sample to an electrophoresis gel and subjecting the gel to an electric field. In an additional embodiment, the present invention relates to methods of separating two or more molecular species present in a sample by gel electrophoresis, comprising applying marker molecule compositions of the present invention to the gel, separately applying the sample to the gel and subjecting the gel to an electric field.

In a further embodiment, the present invention relates to methods further comprising detecting one or more marker molecules and comparing the position of one or more marker molecules to the position of the one or more molecular species after subjecting both to conditions which favor separation based on a physical property of the species.

In one embodiment, the present invention relates to methods further comprising detecting one or more marker molecules and comparing the position of one or more marker molecules to the position of the one or more molecular species after subjecting the gel to an electric field.

In yet another embodiment, the present invention relates to methods of separating two or more molecular species present in a sample, comprising adding the marker molecule compositions of the present invention to the sample containing one or more molecular species, applying the sample to a matrix, and separating the one or more species. In an additional embodiment, the present invention relates to methods of separating two or more molecular species present in a sample, comprising applying marker molecule compositions of the present invention and the sample to a matrix, and separating the one or more species.

In a further embodiment, the present invention relates to a method of characterizing one or more molecular species, comprising:

(a) separating (e.g., by gel electrophoresis in the presence of SDS, HPLC, FPLC, etc.) two or more molecular species and separating at least one (e.g., two, three, four, five, six, seven, eight, nine, ten, eleven, twelve, thirteen, fourteen, fifteen, twenty, etc.) marker molecule of the present invention;

(b) comparing the migration of the one or more molecular species with the migration of the at least one marker molecule of the present invention; and

(c) optionally, determining at least one physical characteristic of the one or more species.

In yet another embodiment, the present invention relates to a method of characterizing one or more molecules of interest, comprising:

(a) electrophoresing or isoelectrically focusing two or more molecules of interest and electrophoresing or isoelectrically focusing at least one (e.g., 1, 2, 3, 4, 5, 6, 7, 8, 9, 10, 11, 12, 13, 14, 15, 20, etc.) marker molecule of the present invention;

(b) comparing the migration of the one or more molecule species with the migration of the at least one marker molecule of the present invention; and

(c) optionally, determining at least one physical characteristic of the one or more species.

In a further embodiment, the present invention relates to a method of characterizing one or more nucleic acids, comprising:

(a) electrophoresing two or more nucleic acids and electrophoresing at least one (e.g., 1, 2, 3, 4, 5, 6, 7, 8, 9, 10, 11, 12, 13, 14, 15, 20, etc.) nucleic acid marker molecule of the present invention;

(b) comparing the migration of the one or more nucleic acids with the migration of the at least one nucleic acid marker molecule of the present invention; and

(c) optionally, determining the size in bp of the one or more nucleic acids.

In one embodiment of this method, a plurality of nucleic acid marker molecules are provided in a composition.

In yet another embodiment, the present invention relates to a method of characterizing one or more proteins, comprising:

(a) electrophoresing or isoelectrically focusing two or more proteins and electrophoresing or isoelectrically focusing at least one (e.g., 1, 2, 3, 4, 5, 6, 7, 8, 9, 10, 11, 12, 13, 14, 15, 20, etc.) protein marker molecule of the present invention;

(b) comparing the migration of the one or more proteins with the migration of the at least one marker molecule of the present invention; and

(c) optionally, determining the isoelectric point (pI) and/or molecular weight of the one or more proteins.

In one embodiment of this method, a plurality of protein marker molecules are provided in a composition.

In a particular aspect, the present invention provides materials and methods for preparing marker molecules in a predictable, ordered and reproducible fashion. Certain methods of the invention involve the modular assembly of multiple nucleic acid molecules to produce product nucleic acid molecules having known or predetermined physical characteristics and/or nucleic acid products encoding proteins having known or predetermined physical characteristics. Because one or more physical characteristics of the nucleic acid products, or the proteins encoded by the nucleic acid products, are known or predetermined, they may be used as standards for determining one or more physical characteristics of the molecular species.

To that end, the present invention provides, for example, materials and methods for preparing marker molecules. In particular embodiments, these methods comprise joining, linking, or combining two or more (e.g., 2, 3, 4, 5, 6, 7, 8, 9, 10, 11, 12, 13, 14, 15, 20, etc.) molecules of nucleic acid, or a segment or portion thereof, by a recombination reaction between recombination sites, at least one of which is present on each nucleic acid molecule or segment being joined, linked, or combined. Such recombination reactions to join multiple nucleic acid molecules, or segments thereof, according to the invention may be conducted in vivo (e.g., within a cell, tissue, organ or organism) or in vitro (e.g., in a cell-free system).

In one embodiment, nucleic acid molecules prepared by recombination reactions of the present invention may themselves be used as nucleic acid markers or may be used as starting molecules (intermediates) to be used in subsequent recombination reactions. Alternatively, in another embodiment, nucleic acid molecules created by the recombination reactions of the present invention may be used to prepare RNA and/or protein markers by transcribing an RNA molecule from a product nucleic acid molecule or by expressing proteins or peptides in vivo or in vitro encoded by the nucleic acid molecules. Nucleic acid molecules created by the recombination reactions may also be used to prepare protein markers by expressing different sequences linked by the methods of the invention to create fusion proteins in vivo or in vitro. Such expression can be accomplished in a cell or by using well known in vitro transcription/translation systems.

In one aspect, at least one (and preferably two or more) nucleic acid molecules, or segments thereof, to be joined by the methods of the invention comprise at least two recombination sites, although each molecule may comprise multiple recombination sites (e.g., two, three, four, five, six, seven, eight, nine, ten, eleven, twelve, thirteen, fourteen, fifteen, twenty, etc.). Such recombination sites (which may be the same or different) may be located at various positions in each nucleic acid molecule or segment.

Nucleic acid molecules of the invention may be of various sizes and may be in different forms including, for example, linear, coiled, closed circles, super-coiled, nicked, double-stranded, single stranded, RNA or DNA.

Nucleic acid molecules used in the invention may also comprise one or more vectors or one or more sequences allowing the molecule to function as a vector in a host cell (such as an origin of replication). Nucleic acid molecules of the invention may also comprise non-coding segments (e.g., intronic, untranslated, or other segments) that serve a structural or other non-expressive functions.

In another aspect, nucleic acid molecules of the invention may be linear molecules having at least one (e.g., 1, 2, 3, 4, 5, 6, 7, 8, 9, 10, 11, 12, 13, 14, 15, 20, etc.) recombination site at or near at least one termini of the molecule and preferably comprise at least one recombination site at or near both termini of the molecule. In yet another aspect, when multiple recombination sites are located on a nucleic acid molecule of interest, such sites do not substantially recombine or do not recombine with each other on that molecule. In this embodiment, the corresponding binding partner recombination sites preferably are located on one or more other nucleic acid molecules to be linked or joined by the methods of the invention. For instance, a first nucleic acid molecule used in the invention may comprise at least a first and second recombination site and a second nucleic acid molecule may comprise at least a third and fourth recombination site, wherein the first and second sites do not substantially recombine with each other and the third and fourth sites do not substantially recombine with each other, although the first and third and/or the second and fourth sites may substantially recombine.

Nucleic acid molecules, or segments thereof, to be joined by methods of the invention (i.e., the “starting molecules”) are used to produce one or more (e.g., two, three, four, five, seven, ten, twelve, fifteen, twenty, thirty, fifty, seventy-five, one hundred, two hundred, etc.) product molecules (e.g., the “product nucleic acid molecules”) containing all or a portion (i.e., a segment) of the starting molecules. The starting molecules can be any nucleic acid molecule derived from any source or produced by any method. Such molecules may be derived from natural sources (such as cells (e.g., prokaryotic cells such as bacterial cells, eukaryotic cells such as fungal cells (e.g., yeast cells), plant cells, animals cells (e.g., mammalian cells such as human cells), etc.), viruses, tissues, organs from any animal or non-animal source, and organisms) or may be non-natural (e.g., derivative nucleic acids) or synthetically derived. Such molecules may also include prokaryotic and eukaryotic vectors, plasmids, integration sequences (e.g., transposons), phage or viral vectors, phagemids, cosmids, and the like. The segments or molecules for use in the invention may be produced by any means known to those skilled in the art including, but not limited to, amplification such as by PCR, isolation from natural sources, chemical synthesis, shearing or restriction digest of larger nucleic acid molecules (such as genomic or cDNA), transcription, reverse transcription and the like, and recombination sites may be added to such molecules by any means known to those skilled in the art including ligation of adapters containing recombination sites, attachment with topoisomerases of adapters containing recombination sites, attachment with topoisomerases of adapter primers containing recombination sites, amplification or nucleic acid synthesis using primers containing recombination sites, insertion or integration of nucleic acid molecules (e.g., transposons or integration sequences) containing recombination sites, etc.

Recombination sites for use in the invention may be any recognition sequence on a nucleic acid molecule which participates in a recombination reaction mediated or catalyzed by one or more recombination proteins. In those embodiments of the present invention utilizing more than one recombination sites, such recombination sites may be the same or different and may recombine with each other or may not recombine or not substantially recombine with each other. Recombination sites useful for purposes of the invention also include mutants, derivatives or variants of wild-type or naturally occurring recombination sites. Recombination site modifications include those that enhance recombination, such enhancements being selected from the group consisting of substantially (i) favoring integrative recombination; (ii) favoring excisive recombination; (iii) relieving the requirement for host factors; (iv) increasing the efficiency of co-integrate or product formation; and (v) increasing the specificity of co-integrate or product formation.

Modifications to the recombination sites include those that enhance recombination specificity, remove one or more stop codons, and/or avoid hair-pin formation. Desired modifications can also be made to the recombination sites to include desired amino acid changes to the transcription or translation product (e.g., mRNA or protein) when translation or transcription occurs across the modified recombination site. Recombination sites used in accordance with the invention include att sites, frt sites, dif sites, psi sites, cer sites, and lox sites or mutants, derivatives and variants thereof (or combinations thereof). Recombination sites contemplated by the invention also include portions of such recombination sites. Depending on the recombination site specificity used, the invention allows directional linking of nucleic acid molecules to provide desired orientations of the linked molecules or non-directional linking to produce random orientations of the linked molecules.

In specific embodiments, the recombination sites which recombine with each other in compositions and used in methods of the invention comprise att sites having identical seven base pair overlap regions. In specific embodiments of the invention, the first three nucleotides of these seven base pair overlap regions comprise nucleotide sequences selected from the group consisting of AAA, AAC, AAG, AAT, ACA, ACC, ACG, ACT, AGA, AGC, AGG, AGT, ATA, ATC, ATG; ATT, CAA, CAC, CAG, CAT, CCA, CCC, CCG, CCT, CGA, CGC, CGG, CGT, CTA, CTC, CTG CTT, GAA, GAC, GAG, GAT, GCA, GCC, GCG, GCT, GGA, GGC, GGG, GGT, GTA, GTC, GTG, GTT, TAA, TAC, TAG, TAT, TCA, TCC, TCG, TCT, TGA, TGC, TGG, TGT, TTA, TTC, TTG, and TTT.

Each starting nucleic acid molecule may comprise, in addition to one or more recombination sites (e.g., two, three, four, five, seven, ten, twelve, fifteen, twenty, thirty, fifty, etc.), a variety of sequences (or combinations thereof) including, but not limited to sequences suitable for use as primer sites (e.g., sequences which a primer such as a sequencing primer or amplification primer may hybridize to initiate nucleic acid synthesis, amplification or sequencing), transcription or translation signals or regulatory sequences such as promoters or enhancers, ribosomal binding sites, Kozak sequences, start codons, transcription and/or translation termination signals such as stop codons (which may be optionally suppressed by one or more suppressor tRNA molecules), origins of replication, selectable markers, and genes or portions of genes which may be used to create protein fusion (e.g., N-terminal or carboxy terminal) such as glutathione S-transferase (GST), histidine tags (HIS6), SUMO-1, maltose binding protein, cellulose binding protein, green fluorescent protein (GFP), yellow fluorescent protein (YFP), cyan fluorescent protein (CFP), blue fluorescent protein (BFP), open reading frame (ORF) sequences, antibody binding domains (e.g. staphylococcal protein A and protein G IgG binding domains), biotin binding domains (e.g. streptavidin, avidin, and domains capable of biotinylation), capture tags (e.g., fluorescein, digoxigenin, FLASH, FLAG, chitin-binding domains), enzymes (e.g. β-galactosidase, β-lactamase, β-glucuronidase (GUS), nucleases, proteases, kinases, phosphatases), a heme group (e.g., cytochrome C, microperoxidase MP-11, horse radish peroxidase) and proteins susceptible to post-translational modification (e.g., phosphorylation (such as β-casein) and glycosylation) and any other sequence of interest which may be desired or used in various molecular biology techniques.

In a further embodiment, nucleic acid molecules of the invention may comprise an open reading frame encoding a binding domain such as complementary determining residues (CDR) of an antibody, a camel antibody, a single-chain antibody, a domain that binds a constant region of an antibody, a domain that binds a nucleic acid (e.g., DNA, RNA, tRNA, ribosomal RNA, mRNA, antisense DNA, a site that regulates gene expression, a nucleic acid from a pathogen, a nucleic acid from a sample, etc.), or a domain that binds a ligand of a receptor. The nucleic acid molecules of the invention may also encode a domain which binds a substance such as a lipid, a small organic molecule, biotin or biotinylated compound, a cell surface receptor or antigen, a crystal or an artificial polymer (e.g., plastic).

In addition, each starting nucleic acid molecule may comprise an open reading frame encoding segments of highly basic proteins such as histones, or segments of proteins encoded by genes from organisms having highly acidic proteins such as Helobacter, or entirely synthetic nucleic acid molecules encoding segments enriched in basic amino acids such as arginine or lysine, or alternatively, segments enriched in acidic amino acids such as aspartic acid or glutamic acid.

In one specific embodiment, the present invention provides nucleic acid molecules in which at least one physical characteristic (e.g., size as determined by the number of bp) of the nucleic acid molecule, or a segment or a portion thereof, is known or predetermined prior to recombination. Thus, product nucleic acid molecules having known or predetermined physical characteristics (e.g., size as determined by the number of bp) may be prepared by selecting starting nucleic acid molecules having known or predetermined physical characteristics. For example, in one aspect, two or more starting nucleic acid molecules may be selected which comprise a nucleic acid segment, L, having a known or predetermined size in bp, wherein said nucleic acid molecules comprise one or more recombination sites. For example, L may comprise 50, 100, 200, 300, 400, 500, 600, 700, 800, 900, 1000, 2000, 2500, 3000, 4000, 5000, 8000, 10,000, 15,000, 20,000, 30,000, 40,000, 50,000, 60,000, or 80,000 or more bp. The recombination sites may be selected so that joining n starting molecules under conditions which favor recombination results in a product nucleic acid molecule comprising (L)ₙ, wherein n is any integer greater than one and equal to or less than one thousand (e.g., 1, 2, 3, 4, 5, 6, 7, 8, 9, 10, 11, 12, 13, 14, 15, 20, 50, 100, etc.). The value of n can be manipulated by the skilled artisan by strategically selecting the number of starting nucleic acid molecules and recombination sites and the specificity of the recombination sites on each starting nucleic acid molecule so that a plurality of product nucleic acid molecules having assorted values of n can be prepared. The resulting product molecule comprising (L)ₙ may be used as a marker molecule itself, or may be used as a starting nucleic acid molecule in another (e.g., second, third, fourth, fifth, sixth, etc.) recombination reaction which joins the product molecule in the first reaction with another starting nucleic acid molecule, such as for example, a vector. In this way, nucleic acid marker molecules may be assembled in a modular fashion by joining together one or more starting molecules having known or predetermined physical properties, e.g., size as determined by the number of basepairs.

In an embodiment, the present invention relates to a method of preparing a product nucleic acid molecule having a known or predetermined physical characteristic comprising:

(a) providing at least two starting nucleic acid molecules, wherein at least one nucleic acid molecule comprises a segment having a known or predetermined physical characteristic and wherein each nucleic acid molecule comprises at least one recombination site capable of recombining with a recombination site present on another segment or another starting nucleic acid molecule;

(b) contacting said nucleic acid molecules under conditions causing recombination between the recombination sites, thereby joining said nucleic acid molecules, or segments thereof, and producing a product nucleic acid molecule; and

(c) isolating said product nucleic acid molecule.

At least two starting nucleic acid molecules are provided in step (a), but the number of starting nucleic acid molecules may be 3, 4, 5, 6, 7, 8, 9, 10, 11, 12, 13, 14, 15, 20 or more. Thus, the product of the recombination reaction may comprise 2, 3, 4, 5, 6, 7, 8, 9, 10, 11, 12, 13, 14, 15, 20, etc. or more segments each corresponding to and derived from a starting nucleic acid molecule.

In some embodiments, two or more of the starting nucleic acid molecules, or segments thereof, may have the same physical characteristic, e.g., they may be identical in size as determined by the number of bp. Thus, for example, multiple starting molecules comprising a nucleic acid segment L, which consists of a predetermined or known or predetermined number of basepairs (for example, 50, 100, 200, 300, 400, 500, 600, 700, 800, 900, 1000, 2000, 2500, 3000, 4000, 5000, 8000, 10,000, 15,000, 20,000, 30,000, 40,000, 50,000, 60,000, and 80,000 or more) may be provided in step (a) resulting in a product nucleic acid molecule which comprises (L)ₙ, wherein n is any integer greater than one.

In another embodiment, the nucleic acid molecules comprising nucleic acid segment L are identical except for the presence of one or more unique recombination sites not present in any other starting nucleic acid molecule.

In yet another embodiment, starting nucleic acid molecules having different physical characteristics (e.g., size as determined by the number of bp) are provided. Thus, a starting nucleic acid molecule comprising segment L may be provided, e.g., with a starting nucleic acid molecule comprising segment B, a starting nucleic acid molecule comprising segment C, a starting nucleic acid molecule comprising segment D, etc. wherein segments L, B, C and D each have a different physical characteristic (e.g., size as determined by the number of bp or encoding different polypeptides). The locations of L, B, C and D within a product nucleic acid molecule may by manipulated by the skilled artisan by strategically selecting recombination sites and the specificity of the recombination sites. Likewise, the number of copies of L, B, C and D within the product nucleic acid molecule may be manipulated by the skilled artisan by strategically selecting the number of starting nucleic acid molecules and recombination sites and the specificity of the recombination sites on each starting nucleic acid molecule. Thus, there may be 1, 2, 3, 4, 5, 6, 7, 8, 9, 10, 11, 12, 13, 14, 15, 20 or more copies of L, B, C and/or D on a product nucleic acid molecule.

In another embodiment, at least one starting nucleic acid molecule can facilitate visualization, isolation or confer other activities to the nucleic acid marker molecule, including for example, the ability to bind proteins or encoding a protein with enzymatic activity. Thus, the present invention provides product nucleic acid molecules comprising B-(L)ₙ, wherein B is a nucleic acid segment which has the ability to bind other proteins, or some other functional activity such as encoding a protein having enzymatic activity. Nucleic acid segment B, may be on the 5′ terminal or 3 terminal end of the product nucleic acid molecule or may be embedded between one or more L nucleic acid segments. In an embodiment, B comprises a region which is capable of binding a protein (e.g., an immunoglobulin such as IgG, an immunoglobulin binding protein such as protein A or protein G, etc). In another embodiment, one or more physical characteristics (e.g., size as determined by the number of bp) of B is known or predetermined. In an additional embodiment, numerous (e.g., two, three, four, five, six, seven, eight, nine, ten, etc.) nucleic acid molecules comprising segment B are provided. Thus, the present invention provides, in another embodiment product nucleic acid molecules comprising (B)ₘ-(L)ₙ, wherein m is an integer greater than 1 and n is an integer greater than 1. Other embodiments related to the above would be apparent to one skilled in the art.

In an additional aspect, the present invention is directed to a method of preparing a product nucleic acid molecule having a known or predetermined physical characteristic comprising:

(a) providing n starting nucleic acid molecules comprising a nucleic acid segment L, each starting nucleic acid molecule comprising at least one recombination site, wherein said recombination sites are chosen such that at least one recombination site is capable of recombining with at least one recombination site of another starting nucleic acid molecule and wherein L is a segment consisting of a known or predetermined number of bp.

(b) contacting said starting nucleic acid molecules under conditions causing recombination between the recombination sites, thereby producing a product nucleic acid molecule comprising (L)ₙ; and

(c) isolating said product nucleic acid molecule.

In a further aspect, the present invention is directed to a method of preparing a product nucleic acid molecule having a known or predetermined physical characteristic comprising:

(a) providing one or more starting nucleic acid molecules comprising a nucleic acid segment B, wherein said nucleic acid molecules each comprise at least one recombination site capable of recombining with at least one recombination site of another starting nucleic acid molecule;

(b) providing n starting nucleic acid molecules comprising a nucleic acid segment L, wherein said nucleic acid molecules each comprise at least one recombination site capable of recombining with at least one recombination site of another starting nucleic acid molecule and wherein L is a segment consisting of a known or predetermined number of bp;

(c) contacting said starting nucleic acid molecules under conditions causing recombination between the recombination sites, thereby joining said segments and producing a product nucleic acid molecule comprising B-(L)ₙ; and

(d) isolating said product nucleic acid molecule.

As an example, three nucleic acid molecules may be provided, each comprising segment L, the first, second, and third nucleic acid molecules all comprising at least one recombination site that flanks segment L. The recombination sites can be chosen so that contacting said starting nucleic acid segments under conditions causing recombination between the recombination sites results in a linear nucleic acid molecule comprising L-L-L. In another embodiment, the recombination sites can be chosen so that contacting said starting nucleic acid molecules under conditions causing recombination between the sites results in a circular nucleic acid molecule comprising L-L-L, wherein the terminal Ls are joined to each other, thereby forming a closed circle.

As another example, four nucleic acid molecules may be provided, each comprising segment L, the first, second, third and fourth nucleic acid molecules all comprising at least one recombination site that flanks segment L. As an example, the recombination sites can be chosen so that contacting said starting nucleic acid segments under conditions causing recombination between the recombination sites results in a linear nucleic acid molecule comprising L-L-L-L. In another embodiment, the recombination sites can be chosen so that contacting said starting nucleic acid molecules under conditions causing recombination between the sites results in a circular nucleic acid molecule comprising L-L-L-L, wherein the terminal Ls are joined to each other, thereby forming a closed circle.

As yet another example, five nucleic acid molecules may be provided, each comprising segment L, the first, second, third, fourth and fifth nucleic acid molecules all comprising at least one recombination site that flanks segment L. As an example, the recombination sites can be chosen so that contacting said starting nucleic acid segments under conditions causing recombination between the recombination sites results in a linear nucleic acid molecule comprising L-L-L-L-L. In another embodiment, the recombination sites can be chosen so that contacting said starting nucleic acid molecules under conditions causing recombination between the sites results in a circular nucleic acid molecule comprising L-L-L-L-L, wherein the terminal Ls are joined to each other, thereby forming a closed circle.

As discussed, the present invention also provides starting nucleic acid molecules comprising regions with the ability to bind other proteins, or some other functional activity such as encoding a protein with enzymatic activity or the ability to bind other proteins. Referring to the above example with four starting nucleic acid molecules, if a fifth nucleic acid segment comprising segment B, which comprises a region having, for example, the ability to bind a protein, is provided, the skilled artisan would be able to select one or more recombination sites to flank segment B such that a product molecule comprising, for example, B-L-L-L-L, L-L-L-L-B, L-B-L-L-L, L-L-B-L-L or L-L-L-B-L is formed.

Once again referring the above example, one of ordinary skill in the art could, using the methods of the invention, prepare product molecules comprising (L)ₙ, and one or more Bs, and one or more other nucleic acid molecules, such as, for example, C and D which are each different from L and B.

In certain embodiments, one or more starting nucleic acid molecules (e.g., 1, 2, 3, 4, 5, 6, 7, 8, 9, 10, 11, 12, 13, 14, 15, 20, etc.), or portions thereof, having at least one recombination site are combined with a target nucleic acid molecule comprising a plurality of recombination sites under conditions which favor recombination.

In another embodiment, one or more starting nucleic acid molecules (e.g., 1, 2, 3, 4, 5, 6, 7, 8, 9, 10, 11, 12, 13, 14, 15, 20, etc.), or portions thereof, having two recombination sites are combined with a target nucleic acid molecule comprising a plurality of recombination sites under conditions which favor recombination.

In yet another embodiment, n starting nucleic acid molecules, which may be the same or different, or portions thereof, wherein n is any integer greater than one, each nucleic acid molecule having two recombination sites are combined with a target nucleic acid molecule comprising 2n recombination sites under conditions which favor recombination.

Thus, in a further embodiment, the invention relates to a method of preparing a product nucleic acid comprising n nucleic acid segments and having a known or predetermined physical characteristic, the method comprising:

(a) providing n nucleic acid segments, each segment flanked by two recombination sites which do not recombine with each other, wherein at least one segment has a known or predetermined physical characteristic;

(b) providing a circularized or linear target nucleic acid molecule comprising 2n recombination sites, wherein each of the 2n recombination sites is capable of recombining with one of the recombination sites flanking one of the nucleic acid segments;

(c) conducting a recombination reaction such that the n nucleic acid segments are recombined into the circularized or linear target nucleic acid thereby producing a product nucleic molecule having n nucleic acid segments; and

(d) isolating said product nucleic acid molecule.

In certain embodiments, the target nucleic acid molecule of step (b) above may be a vector.

Compositions comprising one or more of the nucleic acid molecular marker molecules are also provided in the invention. The compositions may comprise 1, 2, 3, 4, 5, 6, 7, 8, 9, 10, 11, 12, 13, 14, 15, 20, etc. marker molecules. The compositions may also comprise a buffer. The compositions may further comprise marker molecules having the same properties or having an assortment of different properties. For example, in one embodiment, the compositions of the present invention comprise a plurality of nucleic acid marker molecules having an assortment of different sizes as determined by the number of basepairs.

Compositions of the invention may be prepared by repeating the methods for preparing a marker molecule provided above and below to produce a plurality of marker molecules and then combining the plurality of marker molecules in a composition. In addition to a marker molecule or a plurality of marker molecules, the compositions may comprise, for example, water, buffer, sugar (e.g., sucrose, glucose, etc.), antimicrobial agents, dye (e.g., cresol red, ethidium bromide, or others), and molecules capable of binding to the marker molecules bearing labels.

Thus, in one embodiment, the present invention is directed to a method for preparing a composition comprising product nucleic acid molecules having a known or predetermined physical characteristic, said method comprising:

(a) providing at least two starting nucleic acid molecules, wherein at least one nucleic acid molecule comprises a segment having a known or predetermined physical characteristic and wherein each nucleic acid molecule comprises at least one recombination site capable of recombining with a recombination site present on another segment or another starting nucleic acid molecule;

(b) contacting said nucleic acid molecules under conditions causing recombination between the recombination sites, thereby joining said nucleic acid molecules, or segments thereof, and producing a product nucleic acid molecule; and

(c) isolating said product nucleic acid molecule;

(d) repeating steps (a)-(c) one or more times, thereby producing a plurality of product nucleic acid molecules; and

(e) combining the plurality of product nucleic acid molecules thereby preparing said composition.

In another embodiment, the present invention is directed to a method for preparing a composition comprising product nucleic acid molecules having a known or predetermined physical characteristic, said method comprising:

(a) providing n nucleic acid segments, each segment flanked by two recombination sites which do not recombine with each other and wherein at least one segment has a known or predetermined physical characteristic;

(b) providing a circularized or linear target nucleic acid molecule comprising 2n recombination sites, wherein each of the 2n recombination sites is capable of recombining with one of the recombination sites flanking one of the nucleic acid segments;

(c) conducting a recombination reaction such that the n nucleic acid segments are recombined into the circularized or linear target nucleic acid thereby producing a product nucleic molecule having n nucleic acid segments;

(d) isolating said product nucleic acid molecule;

(e) repeating (a)-(d) one or more times, thereby preparing plurality of product nucleic acid molecules; and

(f) combining the plurality of product nucleic acid molecules thereby preparing said composition.

The recombination proteins used in the practice of the invention may comprise one or more proteins selected from the group consisting of Cre, Int, IHF, Xis, Flp, Fis, Hin, Gin, Cin, Tn3 resolvase, TndX, XerC, XerD, and ΦC31. In specific embodiments, the recombination sites comprise one or more recombination sites selected from the group consisting of lox sites; psi sites; dif sites; cer sites; frt sites; att sites; and mutants, variants, and derivatives of these recombination sites which retain the ability to undergo recombination.

Thus, the present invention provides methods for preparing molecular markers, said method comprising joining two or more nucleic acid molecules or segments thereof, under conditions causing the recombination of a recombination site present on one nucleic acid molecule with a recombination site present on the other nucleic acid molecule. The recombination sites are preferably located at or near the ends of the starting nucleic acid molecules. Depending on the location of the recombination sites within the starting molecules, the product molecule thus created will contain all or a portion of the starting molecules joined by a recombination site (which may be a new recombination site). For example, recombination between an attB1 recombination site and an attP1 recombination site results in generation of an attL1 and/or attR1 recombination sites.

In instances where nucleic acid segments joined by methods of the invention contain a terminus, or termini, which do not contain recombination sites, this terminus or termini may be connected to the same nucleic acid segment or another nucleic acid molecule using a ligase or a topoisomerase (e.g., a Vaccinia virus topoisomerase; see U.S. Pat. No. 5,766,891, the entire disclosure of which is incorporated herein by reference).

Further, the invention also provides a means to insert one or more molecules (or combinations thereof) into a product molecule. For instance, using the molecule L-L-L-L described above for illustration, molecule A₁, which comprises one or more recombination sites may be inserted between one or more Ls to form a new molecule, for example, L-L-A1-L-L. In another instance, molecule A₁, may be inserted at the end of the molecule L-L-L-L to form a new molecule, for example, A₁-L-L-L-L.

In one specific embodiment, molecule A₁ is flanked by loxP sites and insertion of molecule A₁ is mediated by Cre recombinase between the loxP sites on the A₁ molecule and corresponding loxP sites on the L molecules. As one skilled in the art would recognize, numerous variations of the above are possible and are included within the scope of the invention. For example, molecule A₂, which comprises one or more recombination sites may be inserted between A₁ and a L or between two Ls form a new molecule comprising, for example, either A₁-A₂-L-L-L-L or A₁-L-L-A₂-L-L, depending on the starting molecule. The methods described herein can be used to insert virtually any number of molecules into other molecules. Further, these methods can be used sequentially, for example, to prepare molecules having physical characteristics.

The product molecules produced by the methods of the invention may comprise any combination of starting molecules (or segments thereof) and can be any size and be in any form (e.g., circular, linear, nicked, supercoiled, etc.), depending on the starting nucleic acid molecule or segment, the location of the recombination sites on the molecule, and the order of recombination of the sites.

The present invention also provides methods for cloning the starting or product nucleic acid molecules of the invention into one or more vectors or converting the starting or product molecules of the invention into one or more vectors. In one aspect, the starting molecules are recombined to make one or more product molecules and such product molecules are cloned (preferably by recombination) into one or more vectors. In another aspect, the starting molecules are cloned directly into one or more vectors such that a number of starting molecules are joined within the vector, thus creating a vector containing the product molecules of the invention. In another aspect, the starting molecules are cloned directly into one or more vectors such that the starting molecules are not joined within the vector (i.e., the starting molecules are separated by vector sequences). In yet another aspect, a combination of product molecules and starting molecules may be cloned in any order into one or more vectors, thus creating a vector comprising a new product molecule resulting from a combination of the original starting and product molecules. Such vectors may be used as nucleic acid marker molecules or may be used as intermediates for the expression of protein marker molecules.

Thus, the invention relates to a method of generating and/or cloning marker molecules or nucleic acid molecules encoding marker molecules comprising:

(a) obtaining at least one (e.g., 1, 2, 3, 4, 5, 6, 7, 8, 9, 10, 11, 12, 13, 14, 15, 20, etc.) nucleic acid molecule of the invention comprising one or more recombination sites; and

(b) transferring all or a portion of said molecule into one or more vectors (e.g., 1, 2, 3, 4, 5, 6, 7, 8, 9, 10, 11, 12, 13, 14, 15, 20, etc.).

Such vectors may comprise one or more recombination sites (e.g., 1, 2, 3, 4, 5, 6, 7, 8, 9, 10, 11, 12, 13, 14, 15, 20, 50, etc.) and the transfer of the molecules into such vectors may be accomplished by recombination between one or more sites on the vectors (e.g., 1, 2, 3, 4, 5, 6, 7, 8, 9, 10, 11, 12, 13, 14, 15, 20, 50, etc.) and one or more sites on the molecules of the invention (e.g., 1, 2, 3, 4, 5, 6, 7, 8, 9, 10, 11, 12, 13, 14, 15, 20, 50, etc.).

Examples of vectors useful in this aspect of the invention are commercially available and include, for example, pT-REx-DEST30 (Invitrogen Corp., Carlsbad, Calif., Cat. No. 12301-016), pT-REx-DEST31 (Invitrogen Corp., Carlsbad, Calif., Cat. No. 12302-014), and pET-DEST42 (Invitrogen Corp., Carlsbad, Calif., Cat. No. 12276-010).

In addition, vectors useful in the present invention may be prepared using particular vector sequences. Thus, in another aspect, the product molecules of the invention may be converted to molecules which function as vectors by including the necessary vector sequences (e.g., origins of replication). Thus, according to the invention, such vectors sequences may be incorporated into the product molecules through the use of starting molecules containing such sequences. Such vector sequences may be added at one or a number of desired locations in the product molecules, depending on the location of the sequence within the starting molecule and the order of addition of the starting molecules in the product molecule. Thus, the invention allows custom construction of a desired vector by combining (preferably through recombination) any number of functional elements that may be desired into the vector. The product molecule containing the vector sequences may be in linear form or may be converted to a circular or supercoiled form by causing recombination between recombination sites within the product molecule or by ligation techniques well known in the art. Circularization of such product molecule may be accomplished by recombining recombination sites at or near both termini of the product molecule or by ligating the termini of the product molecule to circularize the molecule. As will be recognized, linear or circular product molecules can be introduced into one or more hosts or host cells for further manipulation.

Vector sequences useful in the invention, when employed, may comprise one or a number of elements and/or functional sequences and/or sites (or combinations thereof) including one or more sequencing or amplification primers sites (e.g., 1, 2, 3, 4, 5, 6, 7, 8, 9, 10, 11, 12, 13, 14, 15, 20, 50, etc.), one or more sequences which confer translation termination suppressor activities (e.g., 1, 2, 3, 4, 5, 6, 7, 8, 9, 10, 11, 12, 13, 14, 15, 20, 50, etc.) such as sequences which encode suppressor tRNA molecules, one or more selectable markers (e.g., 1, 2, 3, 4, 5, 6, 7, 8, 9, or 10 toxic genes, antibiotic resistance genes, etc.), one or more transcription or translation sites or signals (e.g., 1, 2, 3, 4, 5, 6, 7, 8, 9, 10, 11, 12, 13, 14, 15, 20, 50, etc.), one or more transcription or translation termination sites (e.g., 1, 2, 3, 4, 5, 6, 7, 8, 9, 10, 11, 12, etc.), one or more splice sites (e.g., 1, 2, 3, 4, 5, 6, 7, 8, 9, 10, 11, 12, 13, 14, 15, 20, 50, etc.) which allows for the excision, for example, of RNA corresponding to recombination sites or protein translated from such sites, one or more tag sequences (e.g., HIS6, GST, GUS, GFP, YFP, BFP, CFP, fluorescein, digoxigenin, FLASH, FLAG, chitin-binding domains epitope tags, etc.), one or more restriction enzyme sites (e.g., multiple cloning sites), one or more origins of replication (e.g., 1, 2, 3, 4, 5, 6, 7, 8, 9, 10, etc.), one or more recombination sites (or portions thereof) (e.g., 1, 2, 3, 4, 5, 6, 7, 8, 9, 10, 11, 12, 13, 14, 15, 20, 50, etc.), etc. The vector sequences used in the invention may also comprise stop codons which may be suppressed to allow expression of desired fusion proteins as described herein. Thus, according to the invention, vector sequences may be used to introduce one or more of such elements, functional sequences and/or sites into any of the nucleic acid molecule of the invention, and such sequences may be used to further manipulate or analyze such nucleic acid molecule. For example, primer sites provided by a vector (preferably located on both sides of the insert cloned in such vector) allow sequencing or amplification of all or a portion of a product molecule cloned into the vector.

Additionally, transcriptional or regulatory sequences contained by the vector allows expression of peptides, polypeptides or proteins encoded by all or a portion of the product molecules cloned to the vector. Likewise, genes, portions of genes or sequence tags (such as GUS, GST, GFP, YFP, CFP, His tags, fluorescein, digoxigenin, FLASH, FLAG, chitin-binding domains, epitope tags and the like) provided by the vectors allow creation of populations of gene fusions with the product molecules cloned in the vector or allows production of a number of peptide, polypeptide or protein fusions encoded by the sequence tags provided by the vector in combination with the product sequences cloned in such vector. Such genes, portions of genes or sequence tags may be used in combination with optionally suppressed stop codons to allow controlled expression of fusion proteins encoded by the sequence of interest being cloned into the vector and the vector supplied open reading frame or tag sequence.

In a construct, the vector may comprise one or more recombination sites, one or more stop codons and one or more tag sequences. In some embodiments, the tag sequences may be adjacent to a recombination site. Optionally, a suppressible stop codon may be incorporated into the sequence of the tag or in the sequence of the recombination site in order to allow controlled addition of the tag sequence to the open reading frame of interest. In embodiments of this type, the open reading frame of interest may be inserted into the vector by recombinational cloning such that the tag and the coding sequence of the open reading frame of interest are in the same reading frame.

The open reading frame of interest may be provided with translation initiation signals (e.g., Shine-Delgamo sequences, Kozak sequences and/or IRES sequences) in order to permit the expression of the open reading frame with a native N-terminal when the stop codon is not suppressed. Further, recombination sites which reside between nucleic acid segments which encode components of fusion proteins may be designed either to not encode stop codons or to not encode stop codons in the fusion protein reading frame. The open reading frame of interest may also be provided with a stop codon (e.g., a suppressible stop codon) at the 3′-end of the coding sequence. Similarly, when a fusion protein is produced from multiple nucleic acid segments (e.g., three, four, five, six, eight, ten, etc. segments), nucleic acid which encodes stop codons can be omitted between each nucleic acid segment and, if desired, nucleic acid which encodes a stop codon can be positioned at the 3′ end of the fusion protein coding region.

In some embodiments, a tag sequence may be provided at both the N- and C-termini of the open reading frame of interest. Optionally, the tag sequence at the N-terminus may be provided with a stop codon and the open reading frame of interest may be provided with a stop codon and the tag at the C-terminus may be provided with a stop codon. The stop codons may be the same or different.

In some embodiments, the stop codon of the N-terminal tag is different from the stop codon of the open reading frame of interest. In embodiments of this type, suppressor tRNAs corresponding to one or both of the stop codons may be provided. When both are provided, each of the suppressor tRNAs may be independently provided on the same vector, on a different vector, or in the host cell genome. The suppressor tRNAs need not both be provided in the same way, for example, one may be provided on the vector containing the open reading frame of interest while the other may be provided in the host cell genome.

Depending on the location of the expression signals (e.g., promoters), suppression of the stop codon(s) during expression allows production of a fusion peptide having the tag sequence at the N- and/or C-terminus of the expressed protein. By not suppressing the stop codon(s), expression of the sequence of interest without the N- and/or C-terminal tag sequence may be accomplished. Thus, the invention allows through recombination efficient construction of vectors containing an open reading frame or sequence of interest (e.g., 1, 2, 3, 4, 5, 6, 7, 8, 9, 10 or more ORF's) for controlled expression of fusion proteins depending on the need.

In particular embodiments, the starting nucleic acid molecules or product molecules of the invention which are cloned or constructed according to the invention comprise at least one open reading frame (ORF) (e.g., 1, 2, 3, 4, 5, 6, 7, 8, 9, 10, 11, 12, 13, 14, 15 or more ORFs). Such starting or product molecules may also comprise functional sequences (e.g., primer sites, transcriptional or translation sites or signals, termination sites (e.g., stop codons which may be optionally suppressed), origins of replication, and the like, and in certain aspects comprises sequences that regulate expression of the open reading frame including transcriptional regulatory sequences and sequences that function as internal ribosome entry sites (IRES). At least one of the starting or product molecules and/or vectors may comprise sequences that function as a promoter. Such starting or product molecules and/or vectors may also comprise transcription termination sequences, selectable markers, restriction enzyme recognition sites, and the like.

In some embodiments, the starting or product and/or vectors comprise two copies of the same selectable marker, each copy flanked by two recombination sites. In other embodiments, the starting or product and/or vectors comprise two different selectable markers each flanked by two recombination sites. In some embodiments, one or more of the selectable markers may be a negative selectable marker (e.g., ccdB, kicB, Herpes simplex thymidine kinase, cytosine deaminase, etc.).

The nucleic acid molecules/segments prepared by the methods of the invention can be different types and can have different functions depending on the need and depending on the functional elements present. In one aspect, at least one of the nucleic acid segments cloned according to the invention is operably linked to a sequence which is capable of regulating transcription (e.g., a promoter, an enhancer, a repressor, etc.). For example, at least one of the nucleic acid segments may be operably linked to a promoter which is either an inducible promoter or a constitutive promoter. In yet other specific embodiments, translation of an RNA produced from the cloned nucleic acid segments results in the production of either a fusion protein or all or part of a single protein. In additional specific embodiments, at least one of the nucleic acid segments encodes all of part of an open reading frame and at least one of the nucleic acid segments contains a sequence which is capable of regulating transcription (e.g., a promoter, an enhancer, a repressor, etc.

In specific embodiments of the methods described above, multiple nucleic acid segments are inserted into another nucleic acid molecule. While numerous variations of such methods are possible, in specific embodiments, nucleic acid segments which contain recombination sites having different specificities (e.g., attL1 and attL2) are inserted into a vector which contains more than one set of cognate recombination sites (e.g., attR1 and attR2), each set of which flanks negative selection markers. Thus, recombination at cognate sites results in replacement of the negative selection marker, the loss of which can be used to select for nucleic acid molecules which have undergone recombination at one or more of the recombination sites. The nucleic acid segments which are inserted into the vector may be the same or different. Further, these nucleic acid segments may encode expression products or may be transcriptional control sequences. When the nucleic acid segments encode expression products, vectors of the invention may be used to amplify the copy number or increase expression of encoded products. When the nucleic acid segments encode sequence which regulate transcription (e.g., promoters, enhancers, etc.), vectors of the invention may be used to place multiple regulatory elements in operable linkage with nucleic acid that encodes expression products. Vectors of this nature may be used to increased expression of expression products, for example, by providing multiple binding sites for proteins which activate transcription. Similarly, vectors of this nature may be used to decrease expression of expression products, for example, by providing multiple binding sites for proteins which inhibit transcription. Vectors of this nature may be used to increased or decrease the expression of expression products, for example, by the expression of multiple copies of nucleic acid molecules which encode factors involved in the regulation of transcription. Other embodiments related to the above would be apparent to one skilled in the art.

In another aspect, the present invention provides protein marker molecules prepared by operatively joining two or more starting nucleic acid molecules of the present invention, at least one of which encodes a protein having a known or predetermined physical characteristic, and expressing the encoded protein. As in other embodiments, the nucleic acid molecules in this aspect comprise at least one recombination site and are joined by contacting the nucleic acid molecules under conditions which favor recombination, thereby producing one or more product nucleic acid molecules.

In a further aspect, the present invention provides a multiplicity of protein marker molecules prepared by simultaneous expression of the encoded protein of a multiplicity of nucleic acid molecules of the present invention.

In one embodiment, the starting or product nucleic acid molecules may be cloned into a vector and transformed into host cells in order to express the encoded proteins. In another embodiment, the product nucleic acid molecule may be transformed into a host cell in order to express the encoded proteins. In yet another embodiment, one or more product nucleic acid molecules of a first recombination reaction may be the starting nucleic acid molecule for a second recombination reaction involving linkage of one or more other nucleic acid molecules to the product nucleic acid molecule or molecules of the first recombination reaction. The product nucleic acid molecules of the second recombination reaction may then be used as starting nucleic acid molecules in a third recombination reaction.

Thus, in a further embodiment, the product nucleic acid molecule of any given recombination reaction in a series of recombination reactions may be used as a starting nucleic acid molecule for a subsequent recombination reaction in the series.

In one aspect, the invention provides one or more starting nucleic acid molecules which encode a protein having a known or predetermined physical characteristic such as, for example, molecular weight and/or pI. In another embodiment, one or more starting nucleic acid molecules may encode a protein which is capable of binding another protein or has enzymatic activity. In other embodiments, the starting nucleic acid molecule may not encode for any protein, but may for example be a vector, or other nucleic acid molecule including those comprising sequences suitable for use as primer sites (e.g., sequences which a primer such as a sequencing primer or amplification primer may hybridize to initiate nucleic acid synthesis, amplification or sequencing), transcription or translation signals or regulatory sequences such as promoters or enhancers, ribosomal binding sites, Kozak sequences, start codons, transcription and/or translation termination signals such as stop codons (which may be optimally suppressed by one or more suppressor tRNA molecules), origins of replication, selectable markers. In certain embodiments, the nucleic acid molecules of the invention comprise one or more open reading frames which may be used to create proteins or fusion proteins (e.g., N-terminal or carboxy terminal) such as proteins comprising glutathione S-transferase (GST), β-glucuronidase (GUS), histidine tags (HIS6), SUMO-1, maltose binding protein, cellulose binding protein, green fluorescent protein (GFP), yellow fluorescent protein (YFP), cyan fluorescent protein (CFP), blue fluorescent protein (BFP), open reading frame (ORF) sequences, antibody binding domains (e.g. staphylococcal protein A and protein G IgG binding domains), biotin binding domains (e.g. streptavidin, avidin, and domains capable of biotinylation), capture tags (e.g., fluorescein, digoxigenin, FLASH, FLAG, chitin-binding domains), enzymes (e.g. β-galactosidase, β-lactamase, β-glucuronidase (GUS), nucleases, proteases, kinases, phosphatases), a heme group (e.g., cytochrome C, microperoxidase MP-11, horse radish peroxidase) and proteins susceptible to post-translational modification (e.g., phosphorylation (such as β-casein) and glycosylation).

In a further aspect, starting nucleic acid molecules of the invention may comprise an open reading frame encoding a binding domain such as complementary determining residues (CDR) of an antibody, a camel antibody, a single-chain antibody, a domain that binds a constant region of an antibody, a domain that binds a nucleic acid (e.g., DNA, RNA, tRNA, ribosomal RNA, mRNA, antisense DNA, a site that regulates expression of the open reading frame, a nucleic acid from a pathogen, a nucleic acid from a sample, etc.), or a domain that binds a ligand of a receptor. The nucleic acid molecules of the invention may also encode a domain which binds a substance such as a lipid, a small organic molecule, biotin or biotinylated compound, a cell surface receptor or antigen, a crystal or an artificial polymer (e.g., plastic).

In addition, the present invention provides nucleic acid molecules comprising open reading frames encoding segments of highly basic proteins such as histones, or segments of proteins encoded by genes from organisms having highly acidic proteins such as Helicobacter, or entirely synthetic nucleic acid molecules encoding segments enriched in basic amino acids such as arginine or lysine, or alternatively, segments enriched in acidic amino acids such as aspartic acid or glutamic acid.

In one aspect, the starting nucleic acid molecule may encode different proteins having different physical properties, different proteins having the same physical properties, or the same proteins having the same physical properties.

In an aspect, fusion proteins can be prepared by expressing a product nucleic acid molecule, which has been formed by the in frame linkage of two or more starting nucleic acid molecules, at least two of which encode a protein.

In one embodiment, the present invention provides one or more starting nucleic acid molecules comprising a segment encoding a protein in which at least one physical characteristic (e.g., molecular weight and/or pI) is known or predetermined prior to recombination. Thus, product nucleic acid molecules encoding proteins having known or predetermined physical characteristics may be prepared by strategically selecting starting nucleic acid molecules encoding proteins having known or predetermined physical characteristics. For example, in one aspect, two or more starting nucleic acid molecules may be selected which comprise a segment encoding a protein, M, having a known or predetermined molecular weight in kDa and/or pI, wherein said nucleic acid segment comprises one or more recombination sites. In certain aspects of the invention, M may optionally comprise a region or domain, dM.

The recombination sites may be selected so that joining n starting molecules under conditions which favor recombination results in a product nucleic acid molecule capable of expressing a protein comprising (M)ₙ, wherein n is any integer greater than 1 but less than 1000. The value of n may be, for example, 1, 2, 3, 4, 5, 6, 7, 8, 9, 10, 11, 12, 13, 14, 15, 16, 17, 18, 19, 20, 21, 22, 23, 24, 25, etc. The value of n can be manipulated by the skilled artisan by strategically selecting the number of starting nucleic acid molecules and recombination sites and the specificity of the recombination sites on each starting nucleic acid molecule so that a plurality of product nucleic acid molecules encoding proteins having assorted values of n can be prepared. The resulting product molecule encoding (M)ₙ may be used as a starting nucleic acid molecule in another (e.g., second, third, fourth, fifth, sixth, etc.) recombination reaction which joins the product molecule in the first reaction with another starting nucleic acid molecule. In this way, nucleic acid molecules encoding protein markers may be assembled in a modular fashion by joining together two or more starting molecules encoding proteins having known or predetermined physical properties. After the final recombination reaction, the product nucleic acid molecules may be transformed into host cells and the encoded proteins are expressed.

Alternatively, the product nucleic acid molecules may be used in conjunction with a system in which transcription and translation are coupled in one system, such as an In Vitro Transcription Translation system (IVTT). Such in vitro systems are known in the art. See, e.g., Methods in Molecular Biology, Vol. 37: In Vitro Transcription and Translation Protocols, Edited by: M. J. Tymms, Copyright 1995 Human Press Inc., Totowa, N.J. IVTT systems suitable for use with the present invention are commercially available and include, for example, the Expressway™ In Vitro Protein Synthesis System available from Invitrogen, Corp., Carlsbad, Calif. (Catalog Nos. K9600-01 and K9600-02), the TNT® Coupled Wheat Germ Extract System (catalog number L4140) and TNT® Coupled Reticulocyte Lysate System (catalog number L4610), from Promega Corporation, Madison, Wis.; Retic Lysate IVT™-96 Kit available from Ambion, Inc., Austin, Tex. (Catalog No. 1205), Rapid Translation Systems (RTS) for Protein Expression available from Roche Applied Science (Catalog No. 3186148), and EcoPro™ Extracts from Novagen, Inc. (Catalog No. 70874-3).

In one embodiment, the present invention relates to a method of preparing a protein marker molecule having a known or predetermined physical characteristic, said method comprising:

(a) providing at least two starting nucleic acid molecules, wherein at least one nucleic acid molecule comprises a segment encoding a protein having a known or predetermined physical characteristic and wherein each nucleic acid molecule comprises at least one recombination site capable of recombining with a recombination site present on another segment or starting nucleic acid; and

(b) contacting said starting nucleic acid molecules under conditions causing recombination between the recombination sites, thereby joining said nucleic acid molecules, or said segments, and producing a product nucleic acid molecule which encodes the protein marker molecule.

Optionally, the method further comprises transforming said product nucleic acid molecule into a host cell and causing the product nucleic molecule of (b) to express the encoded protein marker molecule. Alternatively, the method further comprises using the product nucleic acid molecule in conjunction with an IVTT system to express the encoded protein marker molecule. Methods of this type may optionally include purifying the protein.

At least two starting nucleic acid molecules are provided in (a), but the number of starting nucleic acid molecules may be 3, 4, 5, 6, 7, 8, 9, 10, 11, 12, 13, 14, 15, 20, etc. or more. Thus, the product of the recombination reaction may comprise 2, 3, 4, 5, 6, 7, 8, 9, 10, 11, 12, 13, 14, 15, 20, etc. or more segments each corresponding to a starting nucleic acid molecule. It follows then that the product nucleic acid molecule may encode a fusion protein comprising 2, 3, 4, 5, 6, 7, 8, 9, 10, 11, 12, 13, 14, 15, 20, etc. separate protein segments.

In certain embodiments, two or more of the starting nucleic acid molecules, or segments thereof, may encode proteins having the same physical characteristic, e.g., pI and/or molecular weight. Thus, for example, multiple starting molecules comprising a nucleic acid segment encoding protein M, which has a known or predetermined pI or molecular weight (for example, 10, 20, 30, 40, 50, 60, 100, 200, 300, 400, 500, 600, 700, 800, 900, or 1000 kDa) may be provided in (a) resulting in a product nucleic acid molecule which is capable of encoding a protein comprising (M)ₙ, wherein n is any integer greater than zero. As discussed herein, M may comprise a region dM.

In another embodiment, the nucleic acid molecules comprising segments encoding protein M are identical except for the presence of one or more unique recombination sites not present in any other starting nucleic acid molecule.

In another embodiment, at least one starting nucleic acid molecule may encode a protein which facilitates visualization, isolation and/or confers other activities to the protein marker molecule, including for example, enzymatic activity and/or the ability to bind other proteins of interest. Thus, the present invention provides nucleic acid molecules encoding proteins comprising E-(M)ₙ, wherein E is a segment different from M, which has enzymatic activity, the ability to bind other proteins, or some other functional activity. Segment E, may be on the N-terminal or C-terminal end of the protein encoded by the product nucleic acid molecule or may be embedded between one or more M segments. In a one embodiment, E comprises a region which has enzymatic activity, and/or is capable of binding a molecule (e.g. a protein or other molecule). In another embodiment, one or more physical characteristics (e.g., molecular weight and/or pI) of E is known or predetermined. In an additional embodiment, numerous (e.g., 2, 3, 4, 5, 6, 7, 8, 9, 10, 11, 12, 13, 14, 15, 20, etc.) nucleic acid molecules encoding segment E are provided. In yet another embodiment, E may comprise a region dE. Thus, the present invention provides, in another embodiment nucleic acid molecules encoding proteins comprising (E)_{∀}-(M)ₙ, wherein ∀ an integer greater than 1 but less than 1000 and n is an integer greater than 1 but less than 1000. Other embodiments related to the above would be apparent to one skilled in the art.

In an additional aspect, the present invention is directed to a method of preparing a protein marker molecule having a known or predetermined physical characteristic, said method comprising:

(a) providing n starting nucleic acid molecules comprising a nucleic acid segment encoding M, each nucleic acid molecule comprising least one recombination site capable of recombining with at least one recombination site of another starting nucleic acid molecule; and

(b) contacting said starting nucleic acid molecules under conditions causing recombination between the recombination sites, thereby producing a product nucleic acid molecule which encodes a fusion protein marker molecule comprising (M)ₙ.

Optionally, the method further comprises transforming said product nucleic acid molecule into a host cell and causing the product nucleic molecule of (b) to express the encoded protein marker molecule. Alternatively, the method further comprises using the product nucleic acid molecule in conjunction with an IVTT system to express the encoded protein marker molecule. Methods of this type may optionally include purifying the protein.

In a further aspect, the present invention is directed to a method of preparing a protein marker molecule having a known or predetermined physical characteristic, said method comprising:

(a) providing one or more starting nucleic acid molecules comprising a nucleic acid segment encoding E, wherein each of said nucleic acid molecules comprise at least one recombination site capable of recombining with at least one recombination site of another starting nucleic acid molecule;

(b) providing n starting nucleic acid molecules comprising a nucleic acid segment encoding M, wherein each of said nucleic acid molecules comprise at least one recombination site capable of recombining with at least one recombination site of another starting nucleic acid molecule; and

(c) contacting said starting nucleic acid molecules under conditions causing recombination between the recombination sites, thereby producing a product nucleic acid molecule encoding a protein marker molecule comprising E-(M)ₙ.

Optionally, the method further comprises transforming said product nucleic acid molecule into a host cell and causing the product nucleic molecule of (c) to express the encoded protein marker molecule. Alternatively, the method further comprises using the product nucleic acid molecule in conjunction with an IVTT system to express the encoded protein marker molecule. Methods of this type may optionally include purifying the protein.

Thus, if, for example, four starting nucleic acid molecules are provided, each comprising a segment encoding M, the first second, third and fourth nucleic acid molecules all may comprise at least one recombination site that flanks the segment encoding M. As an example, the recombination sites can be chosen so that contacting said starting nucleic acid segments under conditions causing recombination between the recombination sites results in a linear or circular nucleic acid molecule encoding a protein comprising M-M-M-M.

As discussed, the present invention also provides starting nucleic acid molecules comprising segments which encode proteins with enzymatic activity, the ability to bind other proteins, and/or some other functional activity. Referring to the above example, if a fifth nucleic acid segment comprising a segment encoding E is provided, wherein E comprises a region having, for example, the ability to bind a protein, the skilled artisan would be able to strategically select one or more recombination sites for the nucleic acid molecule encoding E such that a product nucleic acid molecule encoding a protein comprising, for example, E-M-M-M-M or M-M-M-M-E or M-E-M-M-M or M-M-E-M-M or M-M-M-E-M is formed.

Once again referring the above example, one of ordinary skill in the art could, using the methods of the invention, prepare product molecules comprising (M)ₙ, and one or more Es, and one or more other proteins, such as, for example, F or G which are each different from M and E. F may comprise a region dF and G may comprise a region dG.

In an additional aspect, the present invention also provides starting nucleic acid molecules encoding a protein M1 or M2 or M3, which have the same molecular weight as protein M, described above, but a different isoelectric point (pI) as protein M. Thus, M can have a pI of one value, for example, 1, 3, 5, 7, 9, 11, 13, and M1 can have a pI of another value, for example, 2, 4, 6, 8, 10, 12 or 14. and M2 can have apI of yet another value, for example, 0.5, 2.5, 4.5, 6.5, 8.5, 10.5, or 12.5, and M3 can have a pI of a further value, for example, 1.5, 3.5, 5.5, 7.5, 9.5, 11.5, or 13.5.

In another aspect, the present invention also provides starting nucleic acid molecules encoding a protein F1 or F2 or F3, which have the same molecular weight as protein F, described above, but a different isoelectric point (pI) as F. In yet another aspect, the present invention also provides starting nucleic acid molecules encoding a protein G1 or G2 or G3, which have the same molecular weight as protein G, described above, but a different isoelectric point (pI) as G.

Thus, as an example, M1 may be a protein having a molecular weight of about 20 kDa and a pI of about 5.0, M2 may be a protein having a molecular weight of about 20 kDa and a pI of about 6.0, and M3 may be a protein having a molecular weight of about 20 kDa and a pI of about 7.0. F1 may be a protein having a molecular weight of about 25 kDa and a pI of 5.0, F2 may be a protein having a molecular weight of about 25 kDa and a pI of about 6.0 and F3 may be a protein having a molecular weight of about 25 kDa and a pI of about 7.0. Using methods described herein, one of ordinary skill in the art would be able to generate nucleic acid molecules encoding fusion proteins comprising, for example, M1-M2, or M2-M3, or M1-M2-M3. Using the methods described herein, one of ordinary skill in the art would also be able to generate nucleic acid molecules encoding fusion proteins, comprising, for example, M1-F1, or M1-F2, or M1-F3. As exemplified above, one of ordinary skill in the art would be able to use the methods and the materials of the present invention to strategically generate nucleic acid molecules encoding proteins having known or predetermined physical characteristics, e.g., molecular weight and/or pI.

In an embodiment, the present invention provides starting nucleic acid molecules encoding a protein consisting of all twenty naturally occurring amino acids. In an additional embodiment, the present invention provides starting nucleic acid molecules encoding a protein consisting of all the naturally occurring amino acids, except one amino acid may be omitted. The omitted amino acid may be alanine, valine, leucine, isoleucine, proline, methionine, phenylalanine, tryptophan, glycine, serine, threonine, cysteine, asparagine, glutamine, tyrosine, aspartate, glutamate, lysine, arginine, or histidine. In an additional embodiment, the present invention provides starting nucleic acid molecules encoding a protein consisting of all the naturally occurring amino acids, except two amino acids may be omitted. The omitted amino acids may be alanine, valine, leucine, isoleucine, proline, methionine, phenylalanine, tryptophan, glycine, serine, threonine, cysteine, asparagine, glutamine, tyrosine, aspartate, glutamate, lysine, arginine, and/or histidine. In an additional embodiment, the present invention provides starting nucleic acid molecules encoding a protein consisting of all the naturally occurring amino acids, except three amino acids may be omitted. The omitted amino acids may be alanine, valine, leucine, isoleucine, proline, methionine, phenylalanine, tryptophan, glycine, serine, threonine, cysteine, asparagine, glutamine, tyrosine, aspartate, glutamate, lysine, arginine, and/or histidine. In an additional embodiment, the present invention provides starting nucleic acid molecules encoding a protein consisting of all the naturally occurring amino acids, except four or more amino acids may be omitted. The omitted amino acids may be alanine, valine, leucine, iso leucine, pro line, methionine, phenylalanine, tryptophan, glycine, serine, threonine, cysteine, asparagine, glutamine, tyrosine, aspartate, glutamate, lysine, arginine, and/or histidine.

In an embodiment, the present invention provides starting nucleic acid molecules encoding a protein consisting of one or more synthetic or non-naturally occurring amino acids.

In certain embodiments, the present invention provides starting nucleic acid molecules encoding a protein consisting of amino acids which do not undergo charge change, e.g., deamination. Thus, for example, the present invention provides starting nucleic acid molecules encoding a protein having no arginine residues. In an additional embodiment, the present invention provides starting nucleic acid molecules encoding a protein consisting of amino acids which do not form disulfide bonds with other amino acids. Thus, for example, the present invention provides starting nucleic acid molecules encoding a protein having no cysteine residues.

In a further embodiment, the present invention provides starting nucleic acid molecules encoding a protein consisting of only positively charged amino acids. For example, the present invention provides starting nucleic acid molecules encoding a protein consisting of only arginine, histidine and/or lysine. In addition, the present invention provides starting nucleic acid molecules encoding a protein consisting of no positively charged amino acids. Thus, for example, the present invention provides starting nucleic acid molecules encoding a protein consisting of any amino acid except arginine, histidine and lysine. In yet an additional embodiment, the present invention provides starting nucleic acid molecules encoding a protein wherein not more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, or 50% of the amino acids are positively charged. In yet an additional embodiment, the present invention provides starting nucleic acid molecules encoding a protein wherein not less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, or 50% of the amino acids are positively charged.

In an additional embodiment, the present invention provides starting nucleic acid molecules encoding a protein consisting of only negatively charged amino acids. For example, the present invention provides starting nucleic acid molecules encoding a protein consisting of only aspartate and/or glutamate. In addition, the present invention provides starting nucleic acid molecules encoding a protein consisting of no negatively charged amino acids. Thus, for example, the present invention provides starting nucleic acid molecules encoding a protein consisting of any amino acid except aspartate and glutamate. In yet an additional embodiment, the present invention provides starting nucleic acid molecules encoding a protein wherein not more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, or 50% of the amino acids are negatively charged. In yet an additional embodiment, the present invention provides starting nucleic acid molecules encoding a protein wherein not less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 40%, or 50% of the amino acids are negatively charged.

In one aspect, the present invention provides a starting nucleic acid molecule encoding M, a starting nucleic acid molecule encoding E, and a starting nucleic acid molecule encoding F, each starting nucleic acid molecule comprising at least one site specific recombination site. The recombination sites can be chosen so that contacting said starting nucleic acid molecules under conditions causing recombination between the recombination sites results in a product nucleic acid capable of encoding a protein comprising M, E and F. If in another recombination reaction a starting nucleic acid molecule encoding M1 instead of M is provided, a product nucleic acid molecule encoding a protein comprising M1, E and F can be prepared. Thus, the present invention provides methods for preparing protein marker molecules having the same molecular weight, but different pI.

In a further aspect, compositions comprising one or more protein molecular marker molecules are also provided in the invention. The compositions may comprise 1, 2, 3, 4, 5, 6, 7, 8, 9, 10, 11, 12, 13, 14, 15, 20, 50, etc., marker molecules. The compositions may comprise marker molecules having the same properties or having and assortment of different properties. For example, in one embodiment, the compositions of the present invention comprise a plurality of protein marker molecules having an assortment of different molecular weights and/or pI.

Compositions comprising one or more of the proteins marker molecules of the invention may be prepared by repeating any of the methods described to produce a protein marker molecule to produce an assortment of protein marker molecules and combining said assortment of protein marker molecules.

In a specific aspect, the invention allows controlled expression of fusion proteins suitable for use as molecular markers by suppression of one or more stop codons. According to the invention, one or more starting molecules (e.g., 1, 2, 3, 4, 5, 6, 7, 8, 9, 10, 11, 12, 13, 14, 15, 20, etc.) joined by the invention may comprise one or more stop codons which may be suppressed to allow expression from a first starting molecule through the next joined starting molecule. For example, a first-second-third starting molecule joined by the invention (when each of such first and second molecules contains a stop codon) can express a tripartite fusion protein encoded by the joined molecules by suppressing each of the stop codons. Moreover, the invention allows selective or controlled fusion protein expression by varying the suppression of selected stop codons. Thus, by suppressing the stop codon between the first and second molecules but not between the second and third molecules of the first-second-third molecule, a fusion protein encoded by the first and second molecule may be produced rather than the tripartite fusion. Thus, use of different stop codons and variable control of suppression allows production of various fusion proteins or portions thereof encoded by all or different portions of the joined starting nucleic acid molecules of interest. In one aspect, the stop codons may be included anywhere within the starting nucleic acid molecule or within a recombination site contained by the starting molecule. Such stop codons may be located at or near the termini of the starting molecule of interest, although such stop codons may be included internally within the molecule. In another aspect, one or more of the starting nucleic acid molecules may comprise the coding sequence of all or a portion of the target open reading frame or open reading frame of interest wherein the coding sequence is followed by a stop codon. The stop codon may then be followed by a recombination site allowing joining of a second starting molecule. In some embodiments of this type, the stop codon may be optionally suppressed by a suppressor tRNA molecule. The genes coding for the suppressor tRNA molecule may be provided on the same vector comprising the target open reading frame of interest, on a different vector, or in the chromosome of the host cell into which the vector comprising the coding sequence is inserted. In some embodiments, more than one copy (e.g., 2, 3, 4, 5, 6, 7, 8, 9, 10, 11, 12, 13, 14, 15, 20, 50, etc.) of the suppressor tRNA may be provided. In some embodiments, the transcription of the suppressor tRNA may be under the control of a regulatable (e.g., inducible or repressible) promoter.

In one aspect, the present invention provides methods for the simultaneous preparation of multiple protein markers encoded by a single nucleic acid molecule. According to this method, one or more starting nucleic acid molecules joined by the invention may comprise one or more stop codons which may be suppressed to allow expression from a first starting molecule through the next starting molecule. Thus, for example, two or more open reading frames encoding the same or different protein may be joined by the methods of the invention and one or more suppressible stop codons may be present between each open reading frame. By strategically manipulating the transcriptional control elements such as the promoter and open reading frame encoding suppressor tRNA so that under certain conditions, one or more stops codons are not completely suppressed, multiple protein markers may be generated from a single nucleic acid molecule. In an embodiment of this particular method, at least one of the starting nucleic acid molecules encode a tag such as HIS6, SUMO-1, maltose binding protein, cellulose binding protein or a binding domain (e.g., an IgG binding domain, a binding domain from protein A, a binding domain from protein G, etc.).

In another embodiment of the claimed invention, the open reading frames found on a nucleic acid molecule are separated by one or more site specific recombination sites. In yet another example, the open reading frames are flanked by one or more sites specific recombination sites. For example, the open reading frames present on a single nucleic acid molecule may be separated or flanked by by one or more attB sites. Thus, in one embodiment, the present invention provides nucleic acid molecules encoding proteins which comprise amino acid sequences corresponding to the amino acid sequence encoded by the site specific recombination sites. Accordingly, the present invention provides, in one embodiment, nucleic acid molecules comprising regions encoding X, wherein X is the amino acid sequence encoded by site specific recombination site.

In another aspect, the invention relates to a method of expressing one or more fusion proteins suitable for use as a marker comprising:

(a) obtaining two or more starting nucleic acid molecules, each comprising at least one recombination site and at least of which comprises one stop codon (preferably the recombination site and/or stop codon are located at or near a terminus or termini of said first nucleic acid molecules);

(b) causing said starting nucleic acid molecules to recombine through recombination of said recombination sites, thereby producing a product nucleic acid molecule comprising said at least one stop codon and all or a portion of said starting nucleic acid molecules; and

(c) expressing one or more peptides or proteins (e.g., 1, 2, 3, 4, 5, 6, 7, 8, 9, 10, 11, 12, 13, 14, 15, 20, 50, etc.) encoded by said product nucleic acid molecule while suppressing said at least one stop codon.

Further, recombination sites described herein (e.g., recombination sites having various recombination specificities) may contain stop codons in one, two or all three forward or reverse reading frames. Such termination codons may be suppressed as described above. Further, in appropriate instances, such recombination sites may be designed so as to eliminate stop codons in one, two and/or all three forward and/or reverse reading frames.

In another aspect, the invention provides methods of preparing protein marker molecules having a known or predetermined physical characteristic, said method comprising:

(a) providing at least a first nucleic acid molecule comprising a coding sequence followed by a stop codon;

(b) providing at least a second nucleic acid molecule comprising a coding sequence, optionally, followed by a stop codon;

(c) causing recombination such that the nucleic acid molecules are joined;

(d) inserting said joined nucleic acid molecules into a vector to produce modified vectors with the two coding sequences connected in frame;

(e) transforming host cells which express suppressor tRNAs with the modified vectors; and

(f) causing expression of the two coding sequences such that fusion proteins encoded by at least a portion of both of the coding sequences are produced, wherein the nucleic acid molecules of (a) and (b) are each flanked by at least one recombination site.

Further, the fused nucleic acid molecules or the vector may comprise at least one suppressible stop codon (e.g., amber, opal and/or ochre codons). In addition, either the first or second nucleic acid molecule may already be present in the vector prior to application of the methods described above. In specific embodiments of the invention, the vectors and/or host cells comprise genes which encode at least one suppressor tRNA molecule. In other specific embodiments, methods of the invention further comprise transforming the host cell with a nucleic acid molecule comprising genes which encode at least one suppressor tRNA molecule. In yet other specific embodiments, the fusion proteins may comprise N- or C-terminal tags (e.g., β-lactamase, glutathione S-transferase, β-glucuronidase, green fluorescent protein, yellow fluorescent protein, red fluorescent protein, cyan fluorescent protein, maltose binding protein, a six histidine tag, an epitope tag, fluorescein, digoxigenin, FLASH, FLAG, chitin-binding domains, etc.) encoded by at least a portion of the vector.

The invention also relates to a method of expressing one or more fusion proteins suitable for use as a molecular marker (e.g., 1, 2, 3, 4, 5, 6, 7, 8, 9, 10, 11, 12, 13, 14, 15, 20, etc.) comprising:

(a) obtaining at least a first nucleic acid molecule comprising at least one recombination site (preferably the recombination site is located at or near a terminus or termini of said first nucleic acid molecule) and a second nucleic acid molecule comprising at least one recombination site (which is preferably located at or near a terminus or termini of said second nucleic acid molecule);

(b) causing said at least first and second nucleic acid molecules to recombine through recombination of said recombination sites, thereby producing a third nucleic acid molecule comprising all or a portion of said at least first and second molecules; and

(c) expressing one or more peptides or proteins (e.g., 1, 2, 3, 4, 5, 6, 7, 8, 9, 10, 11, 12, 13, 14, 15, 20, etc.) encoded by said third nucleic acid molecule either in vitro or in vivo. In certain such embodiments, at least part of the expressed fusion protein will be encoded by the third nucleic acid molecule and at least another part will be encoded by at least part of the first and/or second nucleic acid molecules. Such a fusion protein may be produced by translation of nucleic acid which corresponds to recombination sites located between the first and second nucleic acid molecules. Thus, fusion proteins may be expressed by “reading through” mRNA corresponding to recombination sites used to connect two or more nucleic acid segments. The invention further includes fusion proteins produced by methods of the invention and mRNA which encodes such fusion proteins.

In one aspect, the product nucleic acid molecules created by the methods of the invention may be preferentially selected and thus separated or isolated from the starting molecules and from undesired product molecules (e.g., cointegrates and/or byproduct molecules). Such selection may be accomplished by assaying or selecting for the presence of a desired nucleic acid fusion (PCR with diagnostic primers) and/or the presence of a desired activity of a protein encoded by the desired nucleic acid fusion. Such selection may also be accomplished by positive and/or negative selection. One or more toxic genes (e.g., two, three, four, five seven, ten, etc.) are preferably used according to the invention in such negative selection scheme.

Any of the product molecules of the invention may be further manipulated, analyzed or used in any number of standard molecular biology techniques or combinations of such techniques (in vitro or in vivo). These techniques include sequencing, amplification, restriction digestion, nucleic acid synthesis, making RNA transcripts (e.g., through transcription of product molecules using RNA promoters such as T7 or SP6 promoters), protein or peptide expression (for example, fusion protein expression, antibody expression, hormone expression etc.), protein-protein interactions (2-hybrid or reverse 2-hybrid analysis), homologous recombination or gene targeting, and combinatorial library analysis and manipulation.

Protein expression according to the invention, may comprise:

(a) obtaining a nucleic acid molecule to be expressed which comprises one or more expression signals (e.g., 1, 2, 3, or 4); and

(b) expressing all or a portion of the nucleic acid molecule under control of said expression signal thereby producing a peptide or protein encoded by said molecule or portion thereof.

In this context, the expression signal may be said to be operably linked to the sequence to be expressed. The protein or peptide expressed can be expressed in a host cell (in vivo), although expression may be conducted in vitro (e.g., in cell-free expression systems) using techniques well known in the art. Upon expression of the protein or peptide, the protein or peptide product may optionally be isolated or purified.

Protein expression, according to the invention, may be facilitated by incorporating one or more transcription or translation signals (e.g., 1, 2, 3, 4, 5, 6, 7, 8, 9, 10, 11, 12, 13, 14, 15, 20, 50, etc.) or regulatory sequences, start codons, termination signals, splice donor/acceptor sequences (e.g., intronic sequences) and the like into the product molecules through the use of starting nucleic acid molecules containing such sequences. Thus, by the methods of the invention, expression sequences may be added at one or a number of desired locations in the product molecules, depending on the location of such sequences within the starting molecule and the order of addition of the starting molecule in the product molecule.

The present invention is also directed to isolated protein marker molecules. For example, the present invention is directed to an isolated polypeptide having the structure:

N-(-[M]ₙ-)-C

wherein:

N is an amino terminus;

C is a carboxy terminus;

- represents any number, including 0, of amino acids arranged in any order;

M is an amino acid sequence comprising a domain dM; and

n is any whole integer.

Optionally, n may equal 1, 2, 3, 4, 5, 6, 7, 8, 9, 10, 11, 12, 13, 14, 15, 16, 17, 18, 19, or 20. In addition, M may comprise an amino acid sequence encoded by a site specific recombination site. dM may be any amino acid sequence or region within M and in certain embodiments, may be an enzymatic domain (e.g., nuclease, a recombinase, a phosphatase, a kinase); a binding domain (e.g., a CDR, a camel antibody, a single-chain antibody, a domain that binds the constant region of an antibody, a domain that binds a nucleic acid (such as RNA, tRNA, ribosomal RNA, mRNA, antisense DNA, DNA, a site that regulates gene expression, a nucleic acid from a pathogen, or a nucleic acid from a sample), a domain that is a ligand of a receptor, domain from protein A, or a domain from protein G), a domain that binds a lipid, a small organic molecule, biotin or a biotinylated compound, a cell surface antigen or receptor, a crystal, or an artificial polymer (e.g., plastics); or a detectable domain (e.g. HIS6, GFP, etc.). Nucleic acid molecules encoding polypeptides having the above structure are also included in the invention.

The present invention is also directed to an isolated polypeptide having the structure:

N-(-[X_{∀}-M-X_{∃}]ₙ-)-C

wherein:

N is an amino terminus;

C is a carboxy terminus;

- represents any number, including 0, of amino acids arranged in any order;

M is an amino acid sequence comprising a domain dM;

X represents any amino acid sequence encoded by a site specific recombination site;

α is 0 or 1;

β is 0 or 1; and

n is any whole integer.

The present invention is also directed to a fusion protein comprising a polypeptide having the structure:

N-Fₓ-[M]-N-G_{y}-C

Wherein:

N is an amino terminus;

C is a carboxyl terminus;

- represents any number, including 0, of amino acids arranged in any order;

M is an amino acid sequence comprising a domain dM;

F and G are distinct or identical amino acid sequences that do not comprise domain dM;

x and y are each independently any integer ≧0, provided that x and y are not both 0; and

n is 0 or any integer ≧1.

Optionally, F may comprise a domain dF and G may comprise a domain dG. In addition, M, F and G may comprise amino acids which are encoded by a site specific recombination site. As with dM, dF and/or dG may be an enzymatic domain (e.g., nuclease, a recombinase, a phosphatase, a kinase); a binding domain (e.g., a CDR, a camel antibody, a single-chain antibody, a domain that binds the constant region of an antibody, a domain that binds a nucleic acid (such as RNA, tRNA, ribosomal RNA, mRNA, antisense DNA, DNA, a site that regulates gene expression, a nucleic acid from a pathogen, or a nucleic acid from a sample), a domain that is a ligand of a receptor, domain from protein A, or a domain from protein G), a domain that binds a lipid, a small organic molecule, biotin or a biotinylated compound, a cell surface antigen or receptor, a crystal, or an artificial polymer (e.g., plastics); or a detectable domain (e.g. HIS6, GFP, etc.). dF and dG may be identical, or may be different and may having differenty binding properties (e.g., bind to different ligands or molecules). Nucleic acid molecules encoding polypeptides having the above structure are also included in the invention.

The present invention is further directed to a fusion protein comprising a polypeptide having the structure:

N-X_{∀}-Fₓ-[X_{∃}-M-X₍]ₙ-G_{y}-X_{,}-C

wherein:

N is an amino terminus;

C is a carboxyl terminus;

- represents any number, including 0, of amino acids arranged in any order;

M is an amino acid sequence comprising a domain dM;

F and G are distinct or identical amino acid sequences that do not comprise domain dM;

X represents any amino acid sequence encoded by a site specific recombination site;

x and y are each independently any integer ≧0, provided that x and y are not both 0;

α is 0 or 1;

β is 0 or 1;

γ is 0 or 1;

ε is 0 or 1; and

n is 0 or any integer ≧1.

In yet another aspect, the present invention is directed to a plurality of polypeptides, each polypeptide having the structure:

N-(-[M]ₙ-)-C

wherein:

N is an amino terminus;

C is a carboxy terminus;

- represents any number, including 0, of amino acids arranged in any order;

M is an amino acid sequence comprising a domain dM; and

n is any whole integer greater than 1.

In certain embodiments of the above, the value of n for specific members of the plurality of polypeptides may range from 2 to about 100, 2 to about 30, 2 to about 25, 2 to about 20, 2 to about 15, 2 to about 10, 2 to about 9, or 2 to about 8. In a particular embodiment, each of the specific members of the plurality of polypeptides are present in equimolar amounts.

In another particular embodiment, the plurality of polypeptides have molecular weights of about 20 kDa, about 30 kDa, about 40 kDa, about 50 kDa, about 60 kDa, about 80 kDa, about 100 kDa, about 120 kDa, and about 200 kDa.

In yet another aspect of the invention, compositions comprising two or polypeptides of the invention are provided. For example, the present invention is directed to a composition comprising two or more polypeptides selected from the group consisting of:

N-(-[M]₂-)-C,

N-(-[M]₃-)-C,

N-(-[M]₄-)-C,

N-(-[M]₅-)-C,

N-(-[M]₆-)-C,

N-(-[M]₇-)-C,

N-(-[M]₈-)-C,

N-(-[M]₉-)-C,

N-(-[M]₁₀-)-C,

N-(-[M]₁₁-)-C,

N-(-[M]₁₂-)-C,

N-(-[M]₁₃-)-C,

N-(-[M]₁₄-)-C,

N-(-[M]₁₅-)-C,

N-(-[M]₁₆-)-C,

N-(-[M]₁₇-)-C,

N-(-[M]₁₈-)-C,

N-(-[M]₁₉-)-C, and

N-(-[M]₂₀-)-C

wherein:

N is an amino terminus;

C is a carboxy terminus;

- represents any number, including 0, of amino acids arranged in any order; and

M is an amino acid sequence comprising a domain dM.

In certain aspects, N-(-[M]₂-)-C has a molecular weight of 20 kD; N-(-[M]₃-)-C has a molecular weight of 30 kD; N-(-[M]₄-)-C has a molecular weight of 40 kD; N-(-[M]₅-)-C has a molecular weight of 50 kD; and N-(-[M]₆-)-C has a molecular weight of 60 kD.

In another aspect, the present invention provides kits comprising one or more of the marker molecules described. Kits serve to expedite the performance of, for example, methods of the invention by providing multiple components and reagents packed together. Further, reagents of these kits can be supplied in pre-measured units so as to increase precision and reliability of the methods. Kits of the present invention will generally comprise a carton such as a box; one or more containers such as boxes, tubes, ampoules, jars, or bags; one or more (e.g., 1, 2, 3, etc.) pre-cast gels and the like; one or more (e.g., 1, 2, 3, etc.) buffers; and instructions for use of kit components.

In one aspect, the present invention relates to marker molecule kits comprising a carrier having in close confinement therein at least one (e.g., 1, 2, 3, 4, 5, etc.) container where the first container comprises at least one (e.g., 1, 2, 3, 4, 5, 6, 7, 8, 9, 10, 11, 12, 13, 14, 15, 20, etc.) marker molecules of the present invention. In yet another embodiment, the marker molecule kit of the present invention further comprises instructions for use of kit components. In a further embodiment, the marker molecule kit of the present invention further comprises one or more (e.g., 1, 2, 3, etc.) pre-cast electrophoresis gels.

In yet another aspect, the present invention relates to marker molecule kits comprising one or more starting nucleic acid molecules having a known or predetermined physical characteristic or encoding a protein having a known or predetermined physical characteristic(s) and optionally, buffers, salts, enzymes, cells, vectors, and other reagents necessary to perform the methods described herein. The kits may further provide instructions that allow one of ordinary skill in the art to create custom protein and/or nucleic acid marker molecules having predetermined and/or known physical characteristics tailored to specific needs.

Thus, for example, the marker molecule kit may provide a plurality of starting nucleic acid molecule, each comprising 50 bp, 100 bp, 250 bp, 500 bp, 1000 bp, or 5000 bp. Provided with instructions with respect to the methods of the present invention and the proper buffers, salts, enzymes, cells, vectors, and other reagents, one of ordinary skill in the art could prepare a plurality of marker molecules having an assortment of different sizes. For example, one of ordinary skill in the art could use the instructions and materials provided in the kit to prepare a 150 bp nucleic acid marker by joining the 50 bp and 100 bp starting nucleic acid molecules, a 750 bp nucleic acid marker by joining the 500 bp and 25 bp starting nucleic acid molecules, a 2500 bp nucleic acid marker by joining two of 1000 bp starting nucleic acid molecules and the 500 bp starting nucleic acid molecule, etc.

Also, in another embodiment, the marker molecule kit may provide a plurality of starting nucleic acid molecules, each encoding a protein having a molecular weight of 10 kDa, 20 kDa, 25 kDa, 100 kDa, or 250 kDa. Provided with instructions with respect to the methods of the present invention and the proper buffers, salts, enzymes, cells, vectors, and other reagents, one of ordinary skill in the art could prepare a plurality of marker molecules having an assortment of different molecular weights. For example, one of ordinary skill in the art could use the instructions and materials provided in the kit to prepare a 30 kDa protein marker by joining the starting nucleic acid molecule encoding the 10 kDa protein and the starting nucleic acid molecule encoding the 20 kDa protein and expressing the encoded fusion protein. One skilled in the art could prepare a 50 kDa protein by joining two starting nucleic acid molecules encoding the 25 kDa protein and expressing the encoded protein, etc.

Other embodiments of the invention will be apparent to one of ordinary skill in light of what is known in the art, the following drawings and description of the invention, and the claims.

### BRIEF DESCRIPTION OF THE FIGURES

FIG. 1 is a schematic representation of the basic recombinational cloning reaction.

FIG. 2 is a schematic representation of the use of the present invention to clone two nucleic acid segments by performing an LR recombination reaction.

FIG. 3 is a schematic representation of the use of the present invention to clone two nucleic acid segments by joining the segments using an LR reaction and then inserting the joined fragments into a Destination Vector using a BP recombination reaction.

FIG. 4 is a schematic representation of the use of the present invention to clone two nucleic acid segments by performing a BP reaction followed by an LR reaction.

FIG. 5 is a schematic representation of two nucleic acid segments having attB sites being cloned by performing a first BP reaction to generate an attL site on one segment and an attR on the other followed by an LR reaction to combine the segments. In variations of this process, P1, P2, and/or P3 can be oligonucleotides or linear stretches of nucleotides.

FIG. 6 is a schematic representation of the cloning of two nucleic acid segments into two separate sites in a Destination Vector using an LR reaction.

FIG. 7 is a schematic representation of the cloning of two nucleic acid segments into two separate sites in a vector using a BP reaction.

FIG. 8 is a schematic representation of the cloning of three nucleic acid segments into three vectors using BP reactions, cloning the three segments into a single vector using an LR reaction, and generating segments separated by attB sites.

FIG. 9 is a schematic representation of the cloning of three nucleic acid segments into a single vector using a BP reaction and generating segments separated by attR sites.

FIG. 10 is a plasmid map showing a construct for providing a C-terminal fusion to a gene of interest. SupF encodes a suppressor function. Thus, when supF is expressed, a GUS-GST fusion protein is produced. In variations of this molecule, GUS and/or GST can be any open reading frame.

FIG. 11 is a plasmid map showing a construct for the production of N- and/or C-terminal fusions of two genes of interest (GENE1 and GENE2). Circled numbers represent amber, ochre, or opal stop codons. Suppression of these stop codons result in expression of fusion tags on the N-terminus, the C-terminus, or both termini. In the absence of suppression, native protein is produced.

FIG. 12 is a schematic representation of the single step insertion of four separate DNA segments into a Destination Vector using LR reactions. In particular, a first DNA segment having an attL1 site at the 5′ end and an attL3 site at the 3′ end is linked to a second DNA segment having an attR3 site at the 5′ end and an attL4 site at the 3′ end. The second DNA segment is then linked to a third DNA segment having an attR4 site at the 5′ end and an attL5 site at the 3′ end. The third DNA segment is then linked to a fourth DNA segment having an attR5 site at the 5′ end and an attL2 site at the 3′ end. Thus, upon reaction with LR CLONASE™, the first, second, third, and fourth DNA segments are inserted into a Destination Vector which contains a ccdB gene flanked by attR1 and attR2 sites. The inserted DNA segments are separated from each other and vector sequences by attB1, attB3, attB4, attB5, and attB2 sites.

FIG. 13 is a schematic representation of the insertion of six separate DNA segments into a vector using a two step, one vector process. In particular, a first DNA segment (DNA-A) having an attL1 site at the 5′ end and an attL3 site at the 3′ end is linked to a second DNA segment (DNA-B) having an attR3 site at the 5′ end and an attL4 site at the 3′ end. The second DNA segment is then linked to a third DNA segment (DNA-C) having an attR4 site at the 5′ end and an attL5 site at the 3′ end. A fourth DNA segment (DNA-D) having an attR1 site at the 5′ end and an attL3 site at the 3′ end is linked to a fifth DNA segment (DNA-E) having an attR3 site at the 5′ end and an attL4 site at the 3′ end. The fifth DNA segment is then linked to a sixth DNA segment (DNA-F) having an attR4 site at the 5′ end and an attL2 site at the 3′ end. The two resulting molecules (i.e., DNA-A-DNA-B-DNA-C and DNA-D-DNA-E-DNA-F) are then inserted into the insertion vector. Each of the above reactions is catalyzed by LR CLONASE™. An LR reaction is also used to insert the joined DNA segments into a Destination Vector which contains a ccdB gene flanked by attR1 and attR2 sites. The inserted DNA segments are separated from each other and the vector by attB1, attB3, attB4, attB5, and attB2 sites.

FIG. 14 is a schematic representation of the insertion of six separate DNA segments into a vector using a two step, two vector process. In particular, a first DNA segment (DNA-A) having an attB1 site at the 5′ end and an attL3 site at the 3′ end is linked to a second DNA segment (DNA-B) having an attR3 site at the 5′ end and an attL4 site at the 3′ end. The second DNA segment is then linked to a third DNA segment (DNA-C) having an attR4 site at the 5′ end and an attB5 site at the 3′ end. The linked DNA segments are then inserted into a vector which contains attP1 and attP5 sites. Further, a fourth DNA segment (DNA-D) having an attB5 site at the 5′ end and an attL3 site at the 3′ end is linked to a fifth DNA segment (DNA-E) having an attR3 site at the 5′ end and an attL4 site at the 3′ end. The fifth DNA segment is then linked to a sixth DNA segment (DNA-F) having an attR4 site at the 5′ end and an attB2 site at the 3′ end. The linked DNA segments are then inserted into a vector which contains attP1 and attP2 sites.

After construction of the two plasmids as described, each of which contains three inserted DNA segments, these plasmids are reacted with LR CLONASE™ to generate another plasmid which contains the six DNA segments flanked by attB sites (i.e., B 1-DNA-A-B3-DNA-B-B4-DNA-C-B5-DNA-D-B3-B1-DNA-E-B4-DNA-F-B2).

FIG. 15A is a schematic representation of an exemplary vector of the invention which contains three inserts, labeled “promoter,” “coding sequence,” and “Kan^{r}.” In this example, the inserted promoter drives expression of the coding sequence. Further, an inserted DNA segment confers resistance to kanamycin upon host cells which contain the vector. As discussed below in more detail, a considerable number of vector components (e.g., a selectable marker (for example a kanamycin resistance gene) cassette, an ori cassette, a promoter cassette, a tag sequence cassette, and the like) can be inserted into or used to construct vectors of the invention.

FIG. 15B is a schematic representation of an exemplary vector of the invention which contains four inserts, labeled “promoter 1,” “coding sequence 1,” “promoter 2,” and “coding sequence 2.” In this example, promoter 1 drives expression of coding sequence 1 and promoter 2 drives expression of coding sequence 2.

FIG. 16A shows a process for linking two nucleic acid segments, A and B. The segments are cloned in two similarly configured plasmids. Each segment is flanked by two recombination sites. One of the recombination sites on each plasmid is capable of reacting with its cognate partner on the other plasmid, whereas the other two recombination sites do not react with any other site present. Each plasmid carries a unique origin of replication which may or may not be conditional. Each plasmid also carries both positive and negative selectable markers (+smX and smY, respectively) to enable selection against, and for elements linked to a particular marker. Lastly, each plasmid carries a third recombination site (loxP in this example), suitably positioned to enable deletion of undesired elements and retention of desired elements. In this example, the two plasmids are initially fused at L2 and R2 via a Gateway LxR reaction. This results in the juxtaposition of segments A and B via a B2 recombination site, and the juxtaposition of sm1 and oriB via a P2 recombination site. The two loxP sites in the backbone that flank a series of plasmid elements are depicted in the second panel. Addition of the Cre protein will resolve the single large plasmid into two smaller ones. One of these will be the desired plasmid which carries the linked A and B segments with oriA now linked to sm2 and +sm4. The other carries a set of dispensable and/or undesirable elements. Transformation of an appropriate host and subsequent imposition of appropriate genetic selections will result in loss of the undesired plasmid, while the desired plasmid is maintained.

FIG. 16B shows a process for linking two chimeric nucleic acid segments, A-B and C-D, constructed as shown above in FIG. 16A. The segments are cloned in two similarly configured plasmids. Each segment is flanked by two recombination sites. One of these on each plasmid is capable of reacting with its cognate partner on the other plasmid, whereas the other two recombination sites do not react with any other site present. In this example, the two plasmids are initially fused at L2 and R2 via a Gateway L×R reaction. This results in the juxtaposition of segments A and B via a B2 recombination site, and the juxtaposition of sm1 and oriB via a P2 recombination site. The two loxP sites in the backbone that flank a series of plasmid elements are depicted in the second panel. Addition of the Cre protein will resolve the single large plasmid into two smaller ones. One of these will be the desired plasmid which carries the linked A-B and C-D segments with oriA now linked to sm2 and +sm4. The other carries a set of dispensable and/or undesirable elements. Transformation of an appropriate host and subsequent imposition of appropriate genetic selections will result in loss of the undesired plasmid, whilst the desired plasmid is maintained.

FIG. 17 is a schematic representation of a system for providing a product to a party.

FIG. 18 provides a schematic representation of a system for advising a party as to the availability of a product.

FIG. 19A shows three PCR products encoding a 40 kDa protein, a 60 kDa protein or a 100 kDa protein, wherein each PCR product was amplified with specific flanking att B sites. The PCR amplified 40 kDa, 60 kDa, and 100 kDa ORFs are recombined with three Donor Vectors in a BP reaction.

FIG. 19B shows three starting nucleic acid molecules comprising a segment encoding a 40 kDa protein, a 60 kDa protein or a 100 kDa protein. The three segments are cloned into a Destination Vector in a LR reaction using site specific recombination. The resulting product nucleic acid molecule, the Expression Clone, encodes a fusion protein having a molecular weight of 200 kDa.

FIG. 20A is a photograph of an agarose gel showing PCR amplified products. The PCR amplified fragments were gel purified and used in BP Clonase reactions. The 40 kDa, 60 kDa and 100 kDa amplified ORFs were 1116 bp (lane 1), 1635 bp (lane 2) and 2700 bp (lane 3) in length, respectively. Lane M contained the 1 kb plus DNA molecular weight marker

FIG. 20B shows a photograph of an agarose gel of digested plasmid DNA isolated from three colonies from each of the transformed BP Clonase reactions. The plasmid DNA were digested with BsrGI and separated in a 1.2% E-Gel for analysis. Each of the digested plasmid DNA demonstrated the predicted linearized DNA length. Lanes 1 to 3 are linearized plasmids of pENTR208-40. pENTR213B-60 linearized plasmids are shown in lanes 4 to 6 and the linearized 100 kDa ORF Entry clones, pENTR214C-100, are shown in lanes 7 to 9. The 1 kb plus DNA molecular weight marker is shown in lane M.

FIG. 21A shows gel analysis of pExp 200 kDa clones. Plasmid DNA from eight independently derived pExp 200 kDa clones were digested with BsrGI and separated on a 1.2% E-Gel. Seven of the eight clones (lanes 2 to 8) showed the predicted restriction enzyme digestion profile. Lane 1 showed an unexpected restriction enzyme digestion profile, so this clone was discarded. Lanes M contained the 1 kb plus DNA molecular weight marker.

FIG. 21B shows a schematic of the 200 kDa expression clone, pExp 200 kDa MagicMark. With the addition of the HP-thioredoxin, V5 and 6×His protein motifs to the fusion protein the expected expressed ORF contained 1956 amino acids and a predicted molecular weight of about 200 kDa.

FIG. 22A shows the expression profiles of the 200 kDa MagicMark clones. TOP10 cells were used to express the seven pExp 200 kDa MagicMark clones. All clones expressed a 200 kDa protein after induction with arabinose (lanes 2 to 8). Lanes 1 and 9 were loaded with lysates of cells not induced with arabinose but bearing the pExp 200 kDa MagicMark plasmid. Lane M was loaded with the SeeBlue Plus2 pre-stained protein molecular weight standard.

FIG. 22B shows the chemiluminescent detection of the 200 kDa MagicMark recombinant protein. The lysates of three expression positive clones (FIG. 22A) were fractionated with a NuPAGE gel and transferred onto a PVDF membrane. Detection of the 200 kDa protein was achieved with an alkaline phosphatase-conjugated secondary antibody and the chemiluminescent substrate CDP-Star. Lanes 1 to 3 contained lysates of expression positive clones. Lane 4 was loaded with lysate of cells not induced with arabinose but bearing the pExp 200 kDa MagicMark plasmid. Lane M contained the MagicMark Western Standard.

### DETAILED DESCRIPTION OF THE INVENTION

Definitions

In the description that follows, a number of terms used in recombinant nucleic acid technology are utilized extensively. In order to provide a clear and more consistent understanding of the specification and claims, including the scope to be given such terms, the following definitions are provided.

Gene: As used herein, the term “gene” refers to a nucleic acid which contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. Of course, as would be clearly apparent to one skilled in the art, the transcriptional and translational machinery required for production of the gene product is not included within the definition of a gene. When the gene encodes an untranslated RNA, it includes the promoter and the nucleic acid which encodes the untranslated RNA.

Structural Gene: As used herein, the phrase “structural gene” refers to a nucleic acid which is transcribed into messenger RNA that is then translated into a sequence of amino acids characteristic of a specific polypeptide.

Host: As used herein, the term “host” refers to any prokaryotic or eukaryotic organism that is a recipient of a replicable expression vector, cloning vector or any nucleic acid molecule. The nucleic acid molecule may contain, but is not limited to, a structural gene, a transcriptional regulatory sequence (such as a promoter, an enhancer, a repressor, and the like) and/or an origin of replication. As used herein, the terms “host,” “host cell,” “recombinant host” and “recombinant host cell” may be used interchangeably. For examples of such hosts, see Maniatis et al., Molecular Cloning: A Laboratory Manual, Cold Spring Harbor Laboratory, Cold Spring Harbor, N.Y. (1982).

Transcriptional Regulatory Sequence: As used herein, the phrase “transcriptional regulatory sequence” refers to a functional stretch of nucleotides contained on a nucleic acid molecule, in any configuration or geometry, that act to regulate the transcription of (1) one or more structural genes (e.g., two, three, four, five, seven, ten, etc.) into messenger RNA or (2) one or more genes into untranslated RNA. Examples of transcriptional regulatory sequences include, but are not limited to, promoters, enhancers, repressors, and the like.

Promoter: As used herein, a promoter is an example of a transcriptional regulatory sequence, and is specifically a nucleic acid generally described as the 5′-region of a gene located proximal to the start codon or nucleic acid which encodes untranslated RNA. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

A wide variety of promoters are known to those skilled in the art and these may be used in the present invention. Examples of suitable promoters include, but are not limited to, viral promoters, such as those from cytomegalovirus, Moloney leukemia virus, and herpes virus as well as promoters from viral long terminal repeats (LTRs) such as Rous sarcoma virus LTR. Other suitable promoters include those from the genes encoding metallothionein, skeletal actin, phosphoenolpyruvate carboxylase, phosphoglycerate, dihydrofolate reductase, and thymidine kinase. Promoters may or may not include operators and/or enhancers, which can be constitutively active such as an immunoglobulin enhancer, or inducible such as SV40 enhancer; and the like. For example, a metallothionein promoter is a constitutively active promoter that also can be induced to a higher level of expression upon exposure to a metal ion such as copper, nickel or cadmium ion. In comparison, a tetracycline (tet) inducible promoter is an example of a promoter that is induced upon exposure to tetracycline, or a tetracycline analog, but otherwise is inactive. A transcriptional regulatory element also can be a tissue specific regulatory element, for example, a muscle cell specific regulatory element, such that expression of an encoded product is restricted to the muscle cells in an individual, or to muscle cells in a mixed population of cells in culture, for example, an organ culture. Muscle cell specific regulatory elements including, for example, the muscle creatine kinase promoter (Sternberg et al., Mol. Cell. Biol. 8:2896-2909, 1988, which is incorporated herein by reference) and the myosin light chain enhancer/promoter (Donoghue et al., Proc. Natl. Acad. Sci., USA 88:5847-5851, 1991, which is incorporated herein by reference) are well known in the art. Other tissue specific promoters, as well as regulatory elements only expressed during particular developmental stages of a cell or organism are well known in the art and may be used in the practice of the present invention.

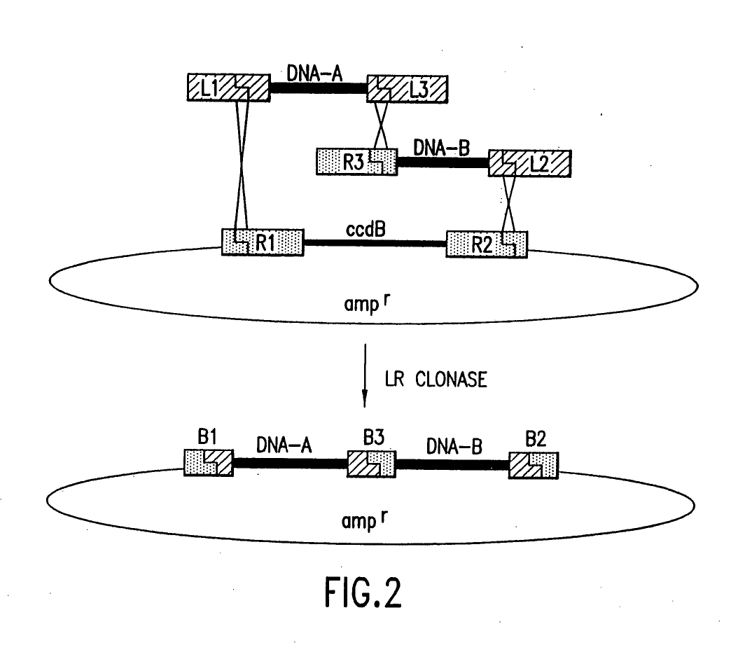

Insert: As used herein, the term “insert” refers to a desired nucleic acid segment that is a part of a larger nucleic acid molecule. In many instances, the insert will be introduced into the larger nucleic acid molecule. For example, the nucleic acid segments labeled ccdB and DNA-A in FIG. 2, are nucleic acid inserts with respect to the larger nucleic acid molecule shown therein. In most instances, the insert will be flanked by recombination sites (e.g., at least one recombination site at each end). In certain embodiments, however, the insert will only contain a recombination site on one end.

Target Nucleic Acid Molecule: As used herein, the phrase “target nucleic acid molecule” refers to a nucleic acid segment of interest, for example, a nucleic acid which is to be acted upon using the compounds and methods of the present invention. Such target nucleic acid molecules may contain one or more gene (e.g., two, three, four, five, seven, ten, twelve, fifteen, twenty, thirty, fifty, etc.) or portions of genes and/or one or more recombination sites. A “target nucleic acid molecule” may also be vector or other nucleic acid which receives and insert nucleic acid molecule.

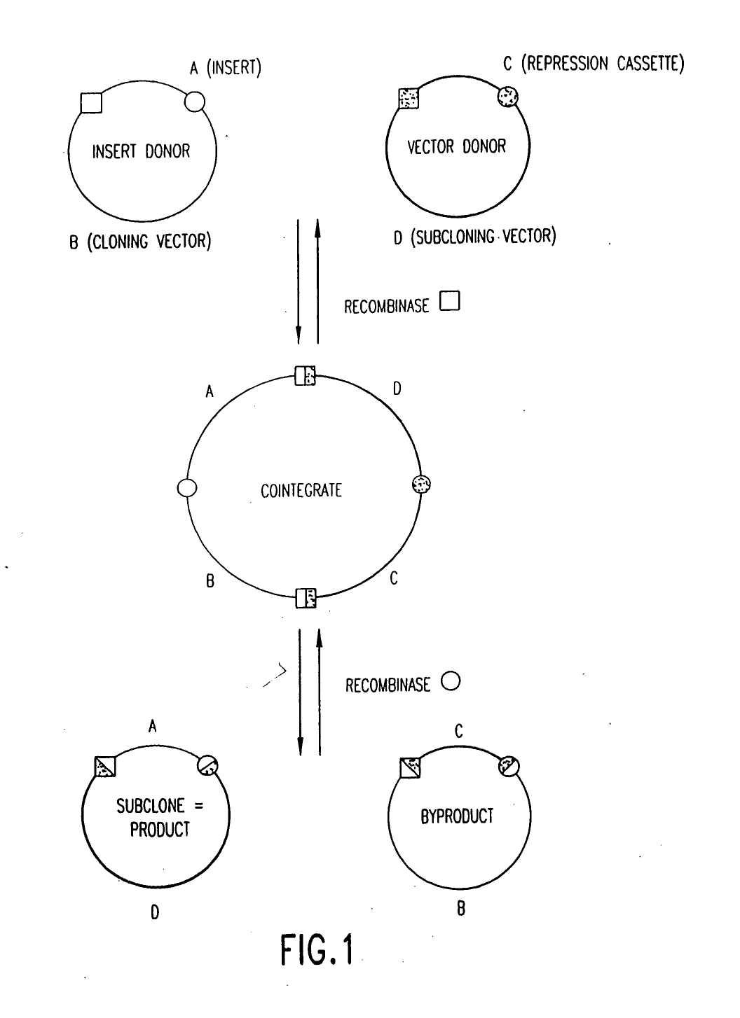

Insert Donor: As used herein, the phrase “Insert Donor” refers to one of the two parental nucleic acid molecules (e.g., RNA or DNA) of the present invention which carries the Insert (see FIG. 1). The Insert Donor molecule comprises the Insert flanked on both sides with recombination sites. The Insert Donor can be linear or circular. In one embodiment of the invention, the Insert Donor is a circular nucleic acid molecule, optionally supercoiled, and further comprises a cloning vector sequence outside of the recombination signals. When a population of Inserts or population of nucleic acid segments are used to make the Insert Donor, a population of Insert Donors result and may be used in accordance with the invention.

Byproduct: As used herein, the term “Byproduct” refers to a daughter molecule (a new clone produced after the second recombination event during the recombinational cloning process) lacking the segment which is desired to be cloned or subcloned.

Cointegrate: As used herein, the term “Cointegrate” refers to at least one recombination intermediate nucleic acid molecule of the present invention that contains both parental (starting) molecules. Cointegrates may be linear or circular. RNA and polypeptides may be expressed from cointegrates using an appropriate host cell strain, for example E. coli DB3.1 (particularly E. coli LIBRARY EFFICIENCY7 DB3.1J Competent Cells), and selecting for both selection markers found on the cointegrate molecule.

Recognition Sequence: As used herein, the phrase “recognition sequence” refers to a particular sequence to which a protein, chemical compound, DNA, or RNA molecule (e.g., restriction endonuclease, a modification methylase, or a recombinase) recognizes and binds. In the present invention, a recognition sequence will usually refer to a recombination site. For example, the recognition sequence for Cre recombinase is loxP which is a 34 base pair sequence comprising two 13 base pair inverted repeats (serving as the recombinase binding sites) flanking an 8 base pair core sequence. (See FIG. 1 of Sauer, B., Current Opinion in Biotechnology 5:521-527 (1994).) Other examples of recognition sequences are the attB, attP, attL, and attR sequences which are recognized by the recombinase enzyme λ Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis). (See Landy, Current Opinion in Biotechnology 3:699-707 (1993).) Such sites may also be engineered according to the present invention to enhance production of products in the methods of the invention. For example, when such engineered sites lack the P1 or HI domains to make the recombination reactions irreversible (e.g., attR or attp), such sites may be designated attR′ or attP′ to show that the domains of these sites have been modified in some way.

Recombination Proteins: As used herein, the phrase “recombination proteins” includes excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Examples of recombination proteins include Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

Recombination Site: A used herein, the phrase “recombination site” refers to a recognition sequence on a nucleic acid molecule which participates in an integration/recombination reaction mediated by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP which is a 34 base pair sequence comprised of two 13 base pair inverted repeats (serving as the recombinase binding sites) flanking an 8 base pair core sequence. (See FIG. 1 of Sauer, B., Curr. Opin. Biotech. 5:521-527 (1994).) Other examples of recognition sequences include the attB, attP, attL, and attR sequences described herein, and mutants, fragments, variants and derivatives thereof, which are recognized by the recombination protein λ Int and by the auxiliary proteins integration host factor (IHF), FIS and excisionase (Xis). (See Landy, Curr. Opin. Biotech. 3:699-707 (1993).)

Recombination sites may be added to molecules by any number of known methods. For example, recombination sites can be added to nucleic acid molecules by blunt end ligation, PCR performed with fully or partially random primers, or inserting the nucleic acid molecules into an vector using a restriction site which flanked by recombination sites.

Recombinational Cloning: As used herein, the phrase “recombinational cloning” refers to a method, such as that described in U.S. Pat. Nos. 5,888,732 and 6,143,557 (the contents of which are fully incorporated herein by reference), whereby segments of nucleic acid molecules or populations of such molecules are exchanged, inserted, replaced, substituted or modified, in vitro or in vivo. Preferably, such cloning method is an in vitro method.

Repression Cassette: As used herein, the phrase “repression cassette” refers to a nucleic acid segment that contains a repressor or a selectable marker present in the subcloning vector.

Selectable Marker: As used herein, the phrase “selectable marker” refers to a nucleic acid segment that allows one to select for or against a molecule (e.g., a replicon) or a cell that contains it, often under particular conditions. These markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products which provide resistance against otherwise toxic compounds (e.g., antibiotics); (2) nucleic acid segments that encode products which are otherwise lacking in the recipient cell (e.g., tRNA genes, auxotrophic markers); (3) nucleic acid segments that encode products which suppress the activity of a gene product; (4) nucleic acid segments that encode products which can be readily identified (e.g., phenotypic markers such as β-galactosidase, green fluorescent protein (GFP), yellow fluorescent protein (YFP), red fluorescent protein (RFP), cyan fluorescent protein (CFP), blue fluorescent protein (BFP) and cell surface proteins); (5) nucleic acid segments that bind products which are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence which can be otherwise non-functional (e.g., for PCR amplification of subpopulations of molecules); (10) nucleic acid segments, which when absent, directly or indirectly confer resistance or sensitivity to particular compounds; and/or (11) nucleic acid segments that encode products which either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, etc.).

Selection Scheme: As used herein, the phrase “selection scheme” refers to any method which allows selection, enrichment, or identification of a desired nucleic acid molecules or host cells contacting them (in particular Product or Product(s) from a mixture containing an Entry Clone or Vector, a Destination Vector, a Donor Vector, an Expression Clone or Vector, any intermediates (e.g., a Cointegrate or a replicon), and/or Byproducts). In one aspect, selection schemes of the invention rely on one or more selectable markers. The selection schemes of one embodiment have at least two components that are either linked or unlinked during recombinational cloning. One component is a selectable marker. The other component controls the expression in vitro or in vivo of the selectable marker, or survival of the cell (or the nucleic acid molecule, e.g., a replicon) harboring the plasmid carrying the selectable marker. Generally, this controlling element will be a repressor or inducer of the selectable marker, but other means for controlling expression or activity of the selectable marker can be used. Whether a repressor or activator is used will depend on whether the marker is for a positive or negative selection, and the exact arrangement of the various nucleic acid segments, as will be readily apparent to those skilled in the art. In some preferred embodiments, the selection scheme results in selection of or enrichment for only one or more desired nucleic acid molecules (such as Products). As defined herein, selecting for a nucleic acid molecule includes (a) selecting or enriching for the presence of the desired nucleic acid molecule (referred to as a “positive selection scheme”), and (b) selecting or enriching against the presence of nucleic acid molecules that are not the desired nucleic acid molecule (referred to as a “negative selection scheme”).

In one embodiment, the selection schemes (which can be carried out in reverse) will take one of three forms, which will be discussed in terms of FIG. 1. The first, exemplified herein with a selectable marker and a repressor therefore, selects for molecules having segment D and lacking segment C. The second selects against molecules having segment C and for molecules having segment D. Possible embodiments of the second form would have a nucleic acid segment carrying a gene toxic to cells into which the in vitro reaction products are to be introduced. A toxic gene can be a nucleic acid that is expressed as a toxic gene product (a toxic protein or RNA), or can be toxic in and of itself. (In the latter case, the toxic gene is understood to carry its classical definition of “heritable trait”.)

Examples of such toxic gene products are well known in the art, and include, but are not limited to, restriction endonucleases (e.g., DpnI, Nla3, etc.); apoptosis-related genes (e.g., ASK1 or members of the bcl-2/ced-9 family); retroviral genes; including those of the human immunodeficiency virus (HIV); defensins such as NP-1; inverted repeats or paired palindromic nucleic acid sequences; bacteriophage lytic genes such as those from ΦX1174 or bacteriophage T4; antibiotic sensitivity genes such as rpsL; antimicrobial sensitivity genes such as pheS; plasmid killer genes; eukaryotic transcriptional vector genes that produce a gene product toxic to bacteria, such as GATA-1; genes that kill hosts in the absence of a suppressing function, e.g., kicB, ccdB, ΦX174 E (Liu, Q. et al., Curr. Biol. 8:1300-1309 (1998)); and other genes that negatively affect replicon stability and/or replication. A toxic gene can alternatively be selectable in vitro, e.g., a restriction site.

Many genes coding for restriction endonucleases operably linked to inducible promoters are known, and may be used in the present invention. (See, e.g., U.S. Pat. No. 4,960,707 (DpnI and DpnII); U.S. Pat. Nos. 5,082,784 and 5,192,675 (KpnI); U.S. Pat. No. 5,147,800 (NgoAIII and NgoAI); U.S. Pat. No. 5,179,015 (FspI and HaeIII): U.S. Pat. No. 5,200,333 (HaeII and TaqI); U.S. Pat. No. 5,248,605 (HpaII); U.S. Pat. No. 5,312,746 (ClaI); U.S. Pat. Nos. 5,231,021 and 5,304,480 (XhoI and XhoII); U.S. Pat. No. 5,334,526 (AluI); U.S. Pat. No. 5,470,740 (NsiI); U.S. Pat. No. 5,534,428 (SstI/SacI); U.S. Pat. No. 5,202,248 (NcoI); U.S. Pat. No. 5,139,942 (NdeI); and U.S. Pat. No. 5,098,839 (PacI). (See also Wilson, G. G., Nucl. Acids Res. 19:2539-2566 (1991); and Lunnen, K. D., et al., Gene 74:25-32 (1988).)

In the second form, segment D carries a selectable marker. The toxic gene would eliminate transformants harboring the Vector Donor, Cointegrate, and Byproduct molecules, while the selectable marker can be used to select for cells containing the Product and against cells harboring only the Insert Donor.

The third form selects for cells that have both segments A and D in cis on the same molecule, but not for cells that have both segments in trans on different molecules. This could be embodied by a selectable marker that is split into two inactive fragments, one each on segments A and D.

The fragments are so arranged relative to the recombination sites that when the segments are brought together by the recombination event, they reconstitute a functional selectable marker. For example, the recombinational event can link a promoter with a structural nucleic acid molecule (e.g., a gene), can link two fragments of a structural nucleic acid molecule, or can link nucleic acid molecules that encode a heterodimeric gene product needed for survival, or can link portions of a replicon.

Site-Specific Recombinase: As used herein, the phrase “site-specific recombinase” refers to a type of recombinase which typically has at least the following four activities (or combinations thereof): (1) recognition of specific nucleic acid sequences; (2) cleavage of said sequence or sequences; (3) topoisomerase activity involved in strand exchange; and (4) ligase activity to reseal the cleaved strands of nucleic acid. (See Sauer, B., Current Opinions in Biotechnology 5:521-527 (1994).) Conservative site-specific recombination is distinguished from homologous recombination and transposition by a high degree of sequence specificity for both partners. The strand exchange mechanism involves the cleavage and rejoining of specific nucleic acid sequences in the absence of DNA synthesis (Landy, A. (1989) Ann. Rev. Biochem. 58:913-949).

Vector: As used herein, the term “vector” refers to a nucleic acid molecule (preferably DNA) that provides a useful biological or biochemical property to an insert. Examples include plasmids, phages, autonomously replicating sequences (ARS), centromeres, and other sequences which are able to replicate or be replicated in vitro or in a host cell, or to convey a desired nucleic acid segment to a desired location within a host cell. A vector can have one or more restriction endonuclease recognition sites (e.g., two, three, four, five, seven, ten, etc.) at which the sequences can be cut in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment which do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. The cloning vector can further contain one or more selectable markers (e.g., two, three, four, five, seven, ten, etc.) suitable for use in the identification of cells transformed with the cloning vector.

Subcloning Vector: As used herein, the phrase “subcloning vector” refers to a cloning vector comprising a circular or linear nucleic acid molecule which includes, preferably, an appropriate replicon. In the present invention, the subcloning vector (segment D in FIG. 1) can also contain functional and/or regulatory elements that are desired to be incorporated into the final product to act upon or with the cloned nucleic acid insert (segment A in FIG. 1). The subcloning vector can also contain a selectable marker (preferably DNA).

Vector Donor: As used herein, the phrase “Vector Donor” refers to one of the two parental nucleic acid molecules (e.g., RNA or DNA) of the present invention which carries the nucleic acid segments comprising the nucleic acid vector which is to become part of the desired Product. The Vector Donor comprises a subcloning vector D (or it can be called the cloning vector if the Insert Donor does not already contain a cloning vector) and a segment C flanked by recombination sites (see FIG. 1). Segments C and/or D can contain elements that contribute to selection for the desired Product daughter molecule, as described above for selection schemes. The recombination signals can be the same or different, and can be acted upon by the same or different recombinases. In addition, the Vector Donor can be linear or circular.

Primer: As used herein, the term “primer” refers to a single stranded or double stranded oligonucleotide that is extended by covalent bonding of nucleotide monomers during amplification or polymerization of a nucleic acid molecule (e.g., a DNA molecule). In one aspect, the primer may be a sequencing primer (for example, a universal sequencing primer). In another aspect, the primer may comprise a recombination site or portion thereof.

Adapter: As used herein, the term “adapter” refers to an oligonucleotide or nucleic acid fragment or segment (preferably DNA) which comprises one or more recombination sites (or portions of such recombination sites) which in accordance with the invention can be added to a circular or linear Insert Donor molecule as well as other nucleic acid molecules described herein. When using portions of recombination sites, the missing portion may be provided by the Insert Donor molecule. Such adapters may be added at any location within a circular or linear molecule, although the adapters are preferably added at or near one or both termini of a linear molecule. Preferably, adapters are positioned to be located on both sides (flanking) a particular nucleic acid molecule of interest. In accordance with the invention, adapters may be added to nucleic acid molecules of interest by standard recombinant techniques (e.g., restriction digest and ligation). For example, adapters may be added to a circular molecule by first digesting the molecule with an appropriate restriction enzyme, adding the adapter at the cleavage site and reforming the circular molecule which contains the adapter(s) at the site of cleavage. In other aspects, adapters may be added by homologous recombination, by integration of RNA molecules, and the like. Alternatively, adapters may be ligated directly to one or more and preferably both termini of a linear molecule thereby resulting in linear molecule(s) having adapters at one or both termini. In one aspect of the invention, adapters may be added to a population of linear molecules, (e.g., a cDNA library or genomic DNA which has been cleaved or digested) to form a population of linear molecules containing adapters at one and preferably both termini of all or substantial portion of said population.

Adapter-Primer: As used herein, the phrase “adapter-primer” refers to a primer molecule which comprises one or more recombination sites (or portions of such recombination sites) which in accordance with the invention can be added to a circular or linear nucleic acid molecule described herein. When using portions of recombination sites, the missing portion may be provided by a nucleic acid molecule (e.g., an adapter) of the invention. Such adapter-primers may be added at any location within a circular or linear molecule, although the adapter-primers are preferably added at or near one or both termini of a linear molecule. Examples of such adapter-primers and the use thereof in accordance with the methods of the invention are shown in Example 8 herein. Such adapter-primers may be used to add one or more recombination sites or portions thereof to circular or linear nucleic acid molecules in a variety of contexts and by a variety of techniques, including but not limited to amplification (e.g., PCR), ligation (e.g., enzymatic or chemical/synthetic ligation), recombination (e.g., homologous or non-homologous (illegitimate) recombination) and the like.

Template: As used herein, the term “template” refers to a double stranded or single stranded nucleic acid molecule which is to be amplified, synthesized or sequenced. In the case of a double-stranded DNA molecule, denaturation of its strands to form a first and a second strand is preferably performed before these molecules may be amplified, synthesized or sequenced, or the double stranded molecule may be used directly as a template. For single stranded templates, a primer complementary to at least a portion of the template hybridizes under appropriate conditions and one or more polypeptides having polymerase activity (e.g., two, three, four, five, or seven DNA polymerases and/or reverse transcriptases) may then synthesize a molecule complementary to all or a portion of the template. Alternatively, for double stranded templates, one or more transcriptional regulatory sequences (e.g., two, three, four, five, seven or more promoters) may be used in combination with one or more polymerases to make nucleic acid molecules complementary to all or a portion of the template. The newly synthesized molecule, according to the invention, may be of equal or shorter length compared to the original template. Mismatch incorporation or strand slippage during the synthesis or extension of the newly synthesized molecule may result in one or a number of mismatched base pairs. Thus, the synthesized molecule need not be exactly complementary to the template. Additionally, a population of nucleic acid templates may be used during synthesis or amplification to produce a population of nucleic acid molecules typically representative of the original template population.

Incorporating: As used herein, the term “incorporating” means becoming a part of a nucleic acid (e.g., DNA) molecule or primer.

Library: As used herein, the term “library” refers to a collection of nucleic acid molecules (circular or linear). In one embodiment, a library may comprise a plurality of nucleic acid molecules (e.g., two, three, four, five, seven, ten, twelve, fifteen, twenty, thirty, fifty, one hundred, two hundred, five hundred one thousand, five thousand, or more), which may or may not be from a common source organism, organ, tissue, or cell. In another embodiment, a library is representative of all or a portion or a substantial portion of the nucleic acid content of an organism (a “genomic” library), or a set of nucleic acid molecules representative of all or a portion or a significant portion of the expressed nucleic acid molecules (a cDNA library or segments derived therefrom) in a cell, tissue, organ or organism. A library may also comprise nucleic acid molecules having random sequences made by de novo synthesis, mutagenesis of one or more nucleic acid molecules, and the like. Such libraries may or may not be contained in one or more vectors (e.g., two, three, four, five, seven, ten, twelve, fifteen, twenty, thirty, fifty, etc.).

Amplification: As used herein, the term “amplification” refers to any in vitro method for increasing the number of copies of a nucleic acid molecule with the use of one or more polypeptides having polymerase activity (e.g., 1, 2, 3, 4 or more nucleic acid polymerases or reverse transcriptases). Nucleic acid amplification results in the incorporation of nucleotides into a DNA and/or RNA molecule or primer thereby forming a new nucleic acid molecule complementary to a template. The formed nucleic acid molecule and its template can be used as templates to synthesize additional nucleic acid molecules. As used herein, one amplification reaction may consist of many rounds of nucleic acid replication. DNA amplification reactions include, for example, polymerase chain reaction (PCR). One PCR reaction may consist of 5 to 100 cycles of denaturation and synthesis of a DNA molecule.

Nucleotide: As used herein, the term “nucleotide” refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Nucleic Acid Molecule: As used herein, the phrase “nucleic acid molecule” refers to a sequence of contiguous nucleotides (riboNTPs, dNTPs or ddNTPs, or combinations thereof) of any length which may encode a full-length polypeptide or a fragment of any length thereof, or which may be non-coding. As used herein, the terms “nucleic acid molecule” and “polynucleotide” may be used interchangeably and include both RNA and DNA.

Oligonucleotide: As used herein, the term “oligonucleotide” refers to a synthetic or natural molecule comprising a covalently linked sequence of nucleotides which are joined by a phosphodiester bond between the 3′ position of the pentose of one nucleotide and the 5′ position of the pentose of the adjacent nucleotide.

Polypeptide: As used herein, the term “polypeptide” refers to a sequence of contiguous amino acids, of any length. The terms “peptide,” “oligopeptide,” or “protein” may be used interchangeably herein with the term “polypeptide.”

Hybridization: As used herein, the terms “hybridization” and “hybridizing” refer to base pairing of two complementary single-stranded nucleic acid molecules (RNA and/or DNA) to give a double stranded molecule. As used herein, two nucleic acid molecules may hybridize, although the base pairing is not completely complementary. Accordingly, mismatched bases do not prevent hybridization of two nucleic acid molecules provided that appropriate conditions, well known in the art, are used. In some aspects, hybridization is said to be under “stringent conditions.” By “stringent conditions,” as the phrase is used herein, is meant overnight incubation at 42EC in a solution comprising: 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65EC.

Other terms used in the fields of recombinant nucleic acid technology and molecular and cell biology as used herein will be generally understood by one of ordinary skill in the applicable arts.

Overview

The present invention relates, in part, to methods of preparing molecular marker molecules and compositions comprising said molecular marker molecules. In particular aspects, these methods involve recombinational joining of two or more segments of nucleic acid molecules resulting in product molecules having a known or predetermined physical characteristic or product molecules encoding proteins having a known or predetermined physical characteristic. Compositions of the invention may comprise a plurality of molecular marker molecules of the invention.

The product nucleic acid molecules produced by the invention may comprise any number of the same or different nucleic acids or other molecules and/or compounds, depending on the starting materials. In addition, deletion or replacement of certain portions or components of the linked products of the invention can be accomplished by recombination (e.g., site-specific recombination).

Recombination Sites

Recombination sites for use in the invention may be any nucleic acid that can serve as a substrate in a recombination reaction. Such recombination sites may be wild-type or naturally occurring recombination sites, or modified, variant, derivative, or mutant recombination sites. Examples of recombination sites for use in the invention include, but are not limited to, phage-lambda recombination sites (such as attP, attB, attL, and attR and mutants or derivatives thereof) and recombination sites from other bacteriophages such as phi80, P22, P2, 186, P4 and P1 (including lox sites such as loxP and loxP511).

Preferred recombination proteins and mutant, modified, variant, or derivative recombination sites for use in the invention include those described in U.S. Pat. Nos. 5,888,732, 6,143,557,6,171,861, 6,270,969, and 6,277,608 and in U.S. application Ser. No. 09/438,358 (filed Nov. 12, 1999), based upon U.S. provisional application No. 60/108,324 (filed Nov. 13, 1998). Mutated att sites (e.g., attB 1-10, attP 1-10, attR 1-10 and attL 1-10) are described in U.S. application Ser. No. 09/517,466, filed Mar. 2, 2000, and Ser. No. 09/732,914, filed Dec. 11, 2000 (published as 2002 0007051-A1) the disclosures of which are specifically incorporated herein by reference in their entirety. Other suitable recombination sites and proteins are those associated with the GATEWAY™ Cloning Technology available from Invitrogen Corporation, Carlsbad, Calif., and described in the product literature of the GATEWAY™ Cloning Technology, the entire disclosures of all of which are specifically incorporated herein by reference in their entireties.

Sites that may be used in the present invention include att sites. The 15 bp core region of the wildtype att site (GCTTTTTTAT ACTAA (SEQ ID NO:1)), which is identical in all wildtype att sites, may be mutated in one or more positions. Other att sites that specifically recombine with other att sites can be constructed by altering nucleotides in and near the 7 base pair overlap region, bases 6-12 of the core region. Thus, recombination sites suitable for use in the methods, molecules, compositions, and vectors of the invention include, but are not limited to, those with insertions, deletions or substitutions of 1, 2, 3, 4, or more nucleotide bases within the 15 base pair core region (see U.S. application Ser. No. 08/663,002, filed Jun. 7, 1996 (now U.S. Pat. No. 5,888,732) and 09/177,387, filed Oct. 23, 1998, which describes the core region in further detail, and the disclosures of which are incorporated herein by reference in their entireties). Recombination sites suitable for use in the methods, compositions, and vectors of the invention also include those with insertions, deletions or substitutions of 1, 2, 3, 4, or more nucleotide bases within the 15 base pair core region that are at least 50% identical, at least 55% identical, at least 60% identical, at least 65% identical, at least 70% identical, at least 75% identical, at least 80% identical, at least 85% identical, at least 90% identical, or at least 95% identical to this 15 base pair core region.

As a practical matter, whether any particular nucleic acid molecule is at least 50%, 60%, 70%, 75%, 80%, 85%, 90%, 95%, 96%, 97%, 98% or 99% identical to, for instance, a given recombination site nucleotide sequence or portion thereof can be determined conventionally using known computer programs such as DNAsis software (Hitachi Software, San Bruno, Calif.) for initial sequence alignment followed by ESEE version 3.0 DNA/protein sequence software (cabot@trog.mbb.sfu.ca) for multiple sequence alignments. Alternatively, such determinations may be accomplished using the BESTFIT program (Wisconsin Sequence Analysis Package, Genetics Computer Group, University Research Park, 575 Science Drive, Madison, Wis. 53711), which employs a local homology algorithm (Smith and Waterman, Advances in Applied Mathematics 2: 482-489 (1981)) to find the best segment of homology between two sequences. When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed. Computer programs such as those discussed above may also be used to determine percent identity and homology between two proteins at the amino acid level.

Analogously, the core regions in attB1, attP1, attL1 and attR1 are identical to one another, as are the core regions in attB2, attP2, attL2 and attR2. Nucleic acid molecules suitable for use with the invention also include those comprising insertions, deletions or substitutions of 1, 2, 3, 4, or more nucleotides within the seven base pair overlap region (TTTATAC, bases 6-12 in the core region). The overlap region is defined by the cut sites for the integrase protein and is the region where strand exchange takes place. Examples of such mutants, fragments, variants and derivatives include, but are not limited to, nucleic acid molecules in which (1) the thymine at position 1 of the seven bp overlap region has been deleted or substituted with a guanine, cytosine, or adenine; (2) the thymine at position 2 of the seven bp overlap region has been deleted or substituted with a guanine, cytosine, or adenine; (3) the thymine at position 3 of the seven bp overlap region has been deleted or substituted with a guanine, cytosine, or adenine; (4) the adenine at position 4 of the seven bp overlap region has been deleted or substituted with a guanine, cytosine, or thymine; (5) the thymine at position 5 of the seven bp overlap region has been deleted or substituted with a guanine, cytosine, or adenine; (6) the adenine at position 6 of the seven bp overlap region has been deleted or substituted with a guanine, cytosine, or thymine; and (7) the cytosine at position 7 of the seven bp overlap region has been deleted or substituted with a guanine, thymine, or adenine; or any combination of one or more (e.g., two, three, four, five, etc.) such deletions and/or substitutions within this seven bp overlap region. The nucleotide sequences of representative seven base pair core regions are set out below.

Altered att sites have been constructed that demonstrate that (1) substitutions made within the first three positions of the seven base pair overlap (TTTATAC) strongly affect the specificity of recombination, (2) substitutions made in the last four positions (TTTATAC) only partially alter recombination specificity, and (3) nucleotide substitutions outside of the seven bp overlap, but elsewhere within the 15 base pair core region, do not affect specificity of recombination but do influence the efficiency of recombination. Thus, nucleic acid molecules and methods of the invention include those comprising or employing 1, 2, 3, 4, 5, 6, 7, 8, 9, 10 or more recombination sites which affect recombination specificity, particularly one or more (e.g., 1, 2, 3, 4, 5, 6, 7, 8, 9, 10, 11, 12, 13, 14, 15, 20, 50, etc.) different recombination sites that may correspond substantially to the seven base pair overlap within the 15 base pair core region, having one or more mutations that affect recombination specificity. Particularly preferred such molecules may comprise a consensus sequence such as NNNATAC wherein “N” refers to any nucleotide (i.e., may be A, G, TIU or C). Preferably, if one of the first three nucleotides in the consensus sequence is a T/U, then at least one of the other two of the first three nucleotides is not a T/U.

The core sequence of each att site (attB, attP, attL and attR) can be divided into functional units consisting of integrase binding sites, integrase cleavage sites and sequences that determine specificity. Specificity determinants are defined by the first three positions following the integrase top strand cleavage site. These three positions are shown with underlining in the following reference sequence: CAACTTTTTTATAC AAAGTTG (SEQ ID NO: 2). Modification of these three positions (64 possible combinations) can be used to generate att sites that recombine with high specificity with other att sites having the same sequence for the first three nucleotides of the seven base pair overlap region. The possible combinations of first three nucleotides of the overlap region are shown in Table 1.

TABLE 1

Modifications of the First Three Nucleotides of
the att Site Seven Base Pair Overlap Region that
Alter Recombination Specificity.

| | | | |
|---|---|---|---|
| AAA | CAA | GAA | TAA |
| AAC | CAC | GAC | TAC |
| AAG | CAG | GAG | TAG |
| AAT | CAT | GAT | TAT |
| ACA | CCA | GCA | TCA |
| ACC | CCC | GCC | TCC |
| ACG | CCG | GCG | TCG |
| ACT | CCT | GCT | TCT |
| AGA | CGA | GGA | TGA |
| AGC | CGC | GGC | TGC |
| AGG | CGG | GGG | TGG |
| AGT | CGT | GGT | TGT |
| ATA | CTA | GTA | TTA |
| ATC | CTC | GTC | TTC |
| ATG | CTG | GTG | TTG |
| ATT | CTT | GTT | TTT |

Representative examples of seven base pair att site overlap regions suitable for in methods, compositions and vectors of the invention are shown in Table 2. The invention further includes nucleic acid molecules comprising one or more (e.g., 1, 2, 3, 4, 5, 6, 7, 8, 9, 10, 11, 12, 13, 14, 15, 20, 50, etc.) nucleotides sequences set out in Table 2. Thus, for example, in one aspect, the invention provides nucleic acid molecules comprising the nucleotide sequence GAAATAC, GATATAC, ACAATAC, or TGCATAC. Table 2. Representative Examples of Seven Base Pair att Site Overlap Regions Suitable for use in the recombination sites of the Invention.

TABLE 2

Representative Examples of Seven Base Pair att
Site Overlap Regions Suitable for use in the
recombination sites of the Invention.

| | | | |
|---|---|---|---|
| AAAATAC | CAAATAC | GAAATAC | TAAATAC |
| AACATAC | CACATAC | GACATAC | TACATAC |
| AAGATAC | CAGATAC | GAGATAC | TAGATAC |
| AATATAC | CATATAC | GATATAC | TATATAC |
| ACAATAC | CCAATAC | GCAATAC | TCAATAC |
| ACCATAC | CCCATAC | GCCATAC | TCCATAC |
| ACGATAC | CCGATAC | GCGATAC | TCGATAC |
| ACTATAC | CCTATAC | GCTATAC | TCTATAC |
| AGAATAC | CGAATAC | GGAATAC | TGAATAC |
| AGCATAC | CGCATAC | GGCATAC | TGCATAC |
| AGGATAC | CGGATAC | GGGATAC | TGGATAC |
| AGTATAC | CGTATAC | GGTATAC | TGTATAC |
| ATAATAC | CTAATAC | GTAATAC | TTAATAC |
| ATCATAC | CTCATAC | GTCATAC | TTCATAC |
| ATGATAC | CTGATAC | GTGATAC | TTGATAC |
| ATTATAC | CTTATAC | GTTATAC | TTTATAC |

As noted above, alterations of nucleotides located 3′ to the three base pair region discussed above can also affect recombination specificity. For example, alterations within the last four positions of the seven base pair overlap can also affect recombination specificity.

For example, mutated att sites that may be used in the practice of the present invention include attB1 (AGCCTGCTTT TTTGTACAAA CTTGT (SEQ ID NO: 3)), attP1 (TACAGGTCAC TAATACCATC TAAGTAGTTG ATTCATAGTG ACTGGATATG TTGTGTTTTA CAGTATTATG TAGTCTGTTT TTTATGCAAA ATCTAATTTA ATATATTGAT ATTTATATCA TTTTACGTTT CTCGTTCAGC TTTTTTGTAC AAAGTTGGCA TTATAAAAAA GCATTGCTCA TCAATTTGTT GCAACGAACA GGTCACTATC AGTCAAAATA AAATCATTAT TTG (SEQ ID NO: 4)), attL1 (CAAATAATGA TTTTATTTTG ACTGATAGTG ACCTGTTCGT TGCAACAAAT TGATAAGCAA TGCTTTTTTA TAATGCCAAC TTTGTACAAA AAAGCAGGCT (SEQ ID NO: 5)), and attR1 (ACAAGTTTGT ACAAAAAAGC TGAACGAGAA ACGTAAAATG ATATAAATAT CAATATATTA AATTAGATTT TGCATAAAAA ACAGACTACA TAATACTGTA AAACACAACA TATCCAGTCA CTATG (SEQ ID NO: 6)). Table 3 provides the sequences of the regions surrounding the core region for the wild type att sites (attB0, P0, R0, and L0) as well as a variety of other suitable recombination sites. Those skilled in the art will appreciated that the remainder of the site may be the same as the corresponding site (B, P, L, or R) listed above.

TABLE 3
Nucleotide sequences of att sites.

| | | |
|---|---|---|
| AttB0 | AGCCTGCTTT TTTATACTAA | (SEQ ID NO: 7) |
| | CTTGAGC | |
| AttP0 | GTTCAGCTTT TTTATACTAA | (SEQ ID NO: 8) |
| | GTTGGCA | |
| AttL0 | AGCCTGCTTT TTTATACTAA | (SEQ ID NO: 9) |
| | GTTGGCA | |
| AttR0 | GTTCAGCTTT TTTATACTAA | (SEQ ID NO: 10) |
| | CTTGAGC | |
| AttB1 | AGCCTGCTTT TTTGTACAAA CTTGT | (SEQ ID NO: 11) |
| AttP1 | GTTCAGCTTT TTTGTACAAA | (SEQ ID NO: 12) |
| | GTTGGCA | |
| AttL1 | AGCCTGCTTT TTTGTACAAA | (SEQ ID NO: 13) |
| | GTTGGCA | |
| AttR1 | GTTCAGCTTT TTTGTACAAA CTTGT | (SEQ ID NO: 14) |
| AttB2 | ACCCAGCTTT CTTGTACAAA GTGGT | (SEQ ID NO: 15) |
| AttP2 | GTTCAGCTTT CTTGTACAAA | (SEQ ID NO: 16) |
| | GTTGGCA | |
| AttL2 | ACCCAGCTTT CTTGTACAAA | (SEQ ID NO: 17) |
| | GTTGGCA | |
| AttR2 | GTTCAGCTTT CTTGTACAAA GTGGT | (SEQ ID NO: 18) |
| AttB5 | CAACTTTATT ATACAAAGTT GT | (SEQ ID NO: 19) |
| AttP5 | GTTCAACTTT ATTATACAAA | (SEQ ID NO: 20) |
| | GTTGGCA | |
| AttL5 | CAACTTTATT ATACAAAGTT GGCA | (SEQ ID NO: 21) |
| AttR5 | GTTCAACTTT ATTATACAAA GTTGT | (SEQ ID NO: 22) |
| AttB11 | CAACTTTTCT ATACAAAGTT GT | (SEQ ID NO: 23) |
| AttP11 | GTTCAACTTT TCTATACAAA | (SEQ ID NO: 24) |
| | GTTGGCA | |
| AttL11 | CAACTTTTCT ATACAAAGTT GGCA | (SEQ ID NO: 25) |
| AttR11 | GTTCAACTTT TCTATACAAA GTTGT | (SEQ ID NO: 26) |
| AttB17 | CAACTTTTGT ATACAAAGTT GT | (SEQ ID NO: 27) |
| AttP17 | GTTCAACTTT TGTATACAAA | (SEQ ID NO: 28) |
| | GTTGGCA | |
| AttL17 | CAACTTTTGT ATACAAAGTT GGCA | (SEQ ID NO: 29) |
| AttR17 | GTTCAACTTT TGTATACAAA GTTGT | (SEQ ID NO: 30) |
| AttB19 | CAACTTTTTC GTACAAAGTT GT | (SEQ ID NO: 31) |
| AttP19 | GTTCAACTTT TTCGTACAAA | (SEQ ID NO: 32) |
| | GTTGGCA | |
| AttL19 | CAACTTTTTC GTACAAAGTT GGCA | (SEQ ID NO: 33) |
| AttR19 | GTTCAACTTT TTCGTACAAA GTTGT | (SEQ ID NO: 34) |
| AttB20 | CAACTTTTTG GTACAAAGTT GT | (SEQ ID NO: 35) |
| AttP20 | GTTCAACTTT TTGGTACAAA | (SEQ ID NO: 36) |
| | GTTGGCA | |
| AttL20 | CAACTTTTTG GTACAAAGTT GGCA | (SEQ ID NO: 37) |
| AttR20 | GTTCAACTTT TTGGTACAAA GTTGT | (SEQ ID NO: 38) |
| AttB21 | CAACTTTTTA ATACAAAGTT GT | (SEQ ID NO: 39) |
| AttP21 | GTTCAACTTT TTAATACAAA | (SEQ ID NO: 40) |
| | GTTGGCA | |
| AttL21 | CAACTTTTTA ATACAAAGTT GGCA | (SEQ ID NO: 41) |
| AttR21 | GTTCAACTTT TTAATACAAA GTTGT | (SEQ ID NO: 42) |

Other recombination sites having unique specificity (i.e., a first site will recombine with its corresponding site and will not substantially recombine with a second site having a different specificity) are known to those skilled in the art and may be used to practice the present invention. Corresponding recombination proteins for these systems may be used in accordance with the invention with the indicated recombination sites. Other systems providing recombination sites and recombination proteins for use in the invention include the FLP/FRT system from Saccharomyces cerevisiae, the resolvase family (e.g., γδ, TndX, TnpX, Tn3 resolvase, Hin, Hjc, Gin, SpCCE1, ParA, and Cin), and IS231 and other Bacillus thuringiensis transposable elements. Other suitable recombination systems for use in the present invention include the XerC and XerD recombinases and the psi, dif and cer recombination sites in E. coli. Other suitable recombination sites may be found in U.S. Pat. No. 5,851,808 issued to Elledge and Liu which is specifically incorporated herein by reference.

The materials and methods of the invention may further encompass the use of “single use” recombination sites which undergo recombination one time and then either undergo recombination with low frequency (e.g., have at least five fold, at least ten fold, at least fifty fold, at least one hundred fold, or at least one thousand fold lower recombination activity in subsequent recombination reactions) or are essentially incapable of undergoing recombination. The invention also provides methods for making and using nucleic acid molecules which contain such single use recombination sites and molecules which contain these sites. Examples of methods which can be used to generate and identify such single use recombination sites are set out below. Further examples of methods which can be used to generate and identify such single use recombination sites are set out in PCT/US00/21623, published as WO 01/11058, which claims priority to U.S. provisional patent application 60/147,892, filed Aug. 9, 1999, both of which are specifically incorporated herein by reference.

The att system core integrase binding site comprises an interrupted seven base pair inverted repeat having the following nucleotide sequence:

| | |
|---|---|
| ------>.......<------ | |
| caactttnnnnnnnaaagttg, | (SEQ ID NO: 43) |

as well as variations thereof which can comprise either perfect or imperfect repeats.

The repeat elements can be subdivided into two distal and/or proximal “domains” composed of caac/gttg segments (underlined), which are distal to the central undefined sequence (the nucleotides of which are represented by the letter “n”), and ttt/aaa segments, which are proximal to the central undefined sequence.

Alterations in the sequence composition of the distal and/or proximal domains on one or both sides of the central undefined region can affect the outcome of a recombination reaction. The scope and scale of the effect is a function of the specific alterations made, as well as the particular recombinational event (e.g., LR vs. BP reactions).

For example, it is believed that an attB site altered to have the following nucleotide sequence:

| | |
|---|---|
| ------>.......<------ | |
| caactttnnnnnnnaaacaag, | (SEQ ID NO: 44) |

will functionally interact with a cognate attP and generate attL and attR. However, whichever of the latter two recombination sites acquires the segment containing “caag” (located on the left side of the sequence shown above) will be rendered non-functional to subsequent recombination events. The above is only one of many possible alterations in the core integrase binding sequence which can render att sites non-functional after engaging in a single recombination event. Thus, single use recombination sites may be prepared by altering nucleotides in the seven base pair inverted repeat regions which abut seven base pair overlap regions of att sites. This region is represented schematically as: CAAC TTT [Seven Base Pair Overlap Region] AAA GTTG.

In generating single use recombination sites, 1, 2, 3, 4, or more of nucleotides of the sequences CAACTTT or AAAGTTG (i.e., the seven base pair inverted repeat regions) may be substituted with other nucleotides or deleted altogether. These seven base pair inverted repeat regions represent complementary sequences with respect to each other. Thus, alterations may be made in either seven base pair inverted repeat region in order to generate single use recombination sites. Further, when DNA is double stranded and one seven base pair inverted repeat region is present, the other seven base pair inverted repeat region will also be present on the other strand.

Using the sequence CAACTTT for illustration, examples of seven base pair inverted repeat regions which can form single use recombination sites include, but are not limited to, nucleic acid molecules in which (1) the cytosine at position 1 of the seven base pair inverted repeat region has been deleted or substituted with a guanine, adenine, or thymine; (2) the adenine at position 2 of the seven base pair inverted repeat region has been deleted or substituted with a guanine, cytosine, or thymine; (3) the adenine at position 3 of the seven base pair inverted repeat region has been deleted or substituted with a guanine, cytosine, or thymine; (4) the cytosine at position 4 of the seven base pair inverted repeat region has been deleted or substituted with a guanine, adenine, or thymine; (5) the thymine at position 5 of the seven base pair inverted repeat region has been deleted or substituted with a guanine, cytosine, or adenine; (6) the thymine at position 6 of the seven base pair inverted repeat region has been deleted or substituted with a guanine, cytosine, or adenine; and (7) the thymine at position 7 of the seven base pair inverted repeat region has been deleted or substituted with a guanine, cytosine, or adenine; or any combination of 1, 2, 3, 4, or more such deletions and/or substitutions within this seven base pair region. Representative examples of nucleotide sequences of the above described seven base pair inverted repeat regions are set out below in Table 4.

TABLE 4

Representative examples of nucleotide sequences of
seven base pair inverted repeat regions.

| | | | |
|---|---|---|---|
| Aagaaaa | aagagcg | aagagaa | aagatat |
| Ccgccac | ccgcctc | ccgcaca | ccgcttt |
| Ggtggga | ggtgctc | ggtgata | ggtgtat |
| Ttctttg | ttctctc | ttctgaa | ttctttt |
| Aatacac | aatagcg | aataaca | aatatat |
| Cctcgga | cctcccg | cctcaca | cctcttt |
| Ggcgaaa | ggcgccg | ggcggaa | ggcgtat |
| Ttgtcac | ttgtgcg | ttgtaca | ttgtttt |
| Acaagga | acaaccg | acaaata | acaattt |
| Caccttg | caccaga | caccgaa | cacctat |
| Gaggcac | gagggcg | gaggaca | gaggttt |
| Tattgga | tattaga | tattaca | tatttat |
| Agaaaaa | agaaaga | agaagaa | agaattt |
| Cgcccac | cgccctc | cgccaca | cgccttt |
| Gcgggga | gcgggcg | gcggata | gcggtat |
| Tcttttg | tcttccg | tcttgaa | tcttttt |
| Ataacac | ataactc | ataaaca | ataattt |
| Ctccaaa | ctccgcg | ctccata | ctcctat |
| Gtgggga | gtggccg | gtgggaa | gtggtat |
| Tgttttg | tgttctc | tgttaca | tgttttt |

Representative examples of nucleotide sequences which form single use recombination sites may also be prepared by combining a nucleotide sequence set out in Table 5, Section 1, with a nucleotide sequence set out in Table 5, Section 2. Single use recombination sites may also be prepared by the insertion of one or more (e.g., 1, 2, 3, 4, 5, 6, 7, etc.) nucleotides internally within these regions.

TABLE 5

Representative examples of nucleotide sequences
which form single use recombination sites.

| | |
|---|---|
| Section 1 (CAAG) | Section 2 (TTT) |

| | | | | | | |
|---|---|---|---|---|---|---|
| Aaaa | cccc | gggg | tttt | aaa | cca | ttc |
| Aaac | ccca | ggga | ttta | aac | cac | ttg |
| Aaag | ccct | gggc | tttc | aag | cgc | tat |
| Aaat | cccg | gggt | tttg | aat | ctc | tct |
| Aaca | ccac | ggag | ttat | aca | ggg | tgt |
| Aaga | ccgc | ggtg | ttct | aga | gga | |
| Aata | cctc | ggcg | ttgt | ata | ggc | |
| Acaa | cacc | gagg | tatt | caa | ggt | |
| Agaa | cgcc | gcgg | tctt | gaa | gag | |
| Ataa | ctcc | gtgg | tgtt | taa | gcg | |
| Caaa | accc | aggg | attt | ccc | gtg | |
| Gaaa | gccc | CGG | cttt | ccg | ttt | |
| Taaa | tccc | tggg | gttt | cct | tta | |

In most instances where one seeks to prevent recombination events with respect to a particular nucleic acid segment, the altered sequence will be located proximally to the nucleic acid segment. Using the following schematic for illustration:

=5′ Nucleic Acid Segment 3′=caac ttt (Seven Base Pair Overlap Region) AAA GTTG,

the lower case nucleotide sequence which represent a seven base pair inverted repeat region (i.e., caac ttt) will generally have a sequence altered by insertion, deletion, and/or substitution to form a single use recombination site when one seeks to prevent recombination at the 3′ end (i.e., proximal end with respect to the nucleic acid segment) of the nucleic acid segment shown. Thus, a single recombination reaction can be used, for example, to integrate the nucleic acid segments into another nucleic acid molecule, then the recombination site becomes effectively non-functional, preventing the site from engaging in further recombination reactions. Similarly, single use recombination sites can be position at both ends of a nucleic acid segment so that the nucleic acid segment can be integrated into another nucleic acid molecule, or circularized, and will remain integrated, or circularized even in the presence of recombinases.

A number of methods may be used to screen potential single use recombination sites for functional activity (e.g., undergo one recombination event followed by the failure to undergo subsequent recombination events). For example, with respect to the screening of recombination sites to identify those which become non-functional after a single recombination event, a first recombination reaction may be performed to generate a plasmid in which a negative selection marker is linked to one or more potentially defective recombination sites. The plasmid may then be reacted with another nucleic acid molecule which comprises a positive selection marker similarly linked to recombination sites. Thus, this selection system is designed such that molecules which recombine are susceptible to negative selection and molecules which do not recombine may be selected for by positive selection. Using such a system, one may then directly select for desired single use core site mutants.

As one skilled in the art would recognize, any number of screening assays may be designed which achieve the same results as those described above. In many instances, these assays will be designed so that an initial recombination event takes place and then recombination sites which are unable to engage in subsequent recombination events are identified or molecules which contain such recombination sites are selected for. A related screening assay would result in selection against nucleic acid molecule which have undergone a second recombination event. Further, as noted above, screening assays can be designed where there is selection against molecules which have engaged in subsequent recombination events and selection for those which have not engaged in subsequent recombination events.

Single use recombination sites are especially useful for either decreasing the frequency of or preventing recombination when either large number of nucleic acid segments are attached to each other or multiple recombination reactions are performed. Thus, the invention further includes nucleic acid molecules which contain single use recombination sites, as well as methods for performing recombination using these sites.

Recombination sites used with the invention may also have embedded functions or properties. An embedded functionality is a function or property conferred by a nucleotide sequence in a recombination site that is not directly associated with recombination efficiency or specificity. For example, recombination sites may contain protein coding sequences (e.g., intein coding sequences), intron/exon splice sites, origins of replication, and/or stop codons. Further, recombination sites that have more than one (e.g., two, three, four, five, etc.) embedded functions or properties may also be prepared.

In some instances it will be advantageous to remove either RNA corresponding to recombination sites from RNA transcripts or amino acid residues encoded by recombination sites from polypeptides translated from such RNAs. Removal of such sequences can be performed in several ways and can occur at either the RNA or protein level. One instance where it may be advantageous to remove RNA transcribed from a recombination site will be when constructing a fusion polypeptide between a polypeptide of interest and a coding sequence present on the vector. The presence of an intervening recombination site between the ORF of the polypeptide of interest and the vector coding sequences may result in the recombination site (1) contributing codons to the mRNA that result in the inclusion of additional amino acid residues in the expression product, (2) contributing a stop codon to the mRNA that prevents the production of the desired fusion protein, and/or (3) shifting the reading frame of the mRNA such that the two protein are not fused “in-frame.”

In one aspect, the invention provides methods for removing nucleotide sequences encoded by recombination sites from RNA molecules. One example of such a method employs the use of intron/exon splice sites to remove RNA encoded by recombination sites from RNA transcripts. Nucleotide sequences that encode intron/exon splice sites may be fully or partially embedded in the recombination sites used in the present invention and/or may encoded by adjacent nucleic acid sequence. Sequences to be excised from RNA molecules may be flanked by splice sites that are appropriately located in the sequence of interest and/or on the vector. For example, one intron/exon splice site may be encoded by a recombination site and another intron/exon splice site may be encoded by other nucleotide sequences (e.g., nucleic acid sequences of the vector or a nucleic acid of interest). Nucleic acid splicing is well known to those skilled in the art and is discussed in the following publications: R. Reed, Curr. Opin. Genet. Devel. 6:215-220 (1996); S. Mount, Nucl. Acids. Res. 10:459-472, (1982); P. Sharp, Cell 77:805-815, (1994); K. Nelson and M. Green, Genes and Devel. 23:319-329 (1988); and T. Cooper and W. Mattox, Am. J. Hum. Genet. 61:259-266 (1997).

Splice sites can be suitably positioned in a number of locations. For example, a Destination Vector designed to express an inserted ORF with an N-terminal fusion—for example, with a detectable marker—the first splice site could be encoded by vector sequences located 3′ to the detectable marker coding sequences and the second splice site could be partially embedded in the recombination site that separates the detectable marker coding sequences from the coding sequences of the ORF. Further, the second splice site either could abut the 3′ end of the recombination site or could be positioned a short distance (e.g., 2, 4, 8, 10, 20 nucleotides) 3′ to the recombination site. In addition, depending on the length of the recombination site, the second splice site could be fully embedded in the recombination site.

A modification of the method described above involves the connection of multiple nucleic acid segments that, upon expression, results in the production of a fusion protein. In one specific example, one nucleic acid segment encodes detectable marker—for example, GFP—and another nucleic acid segment that encodes an ORF of interest. Each of these segments is flanked by recombination sites. In addition, the nucleic acid segments that encodes the detectable marker contains an intron/exon splice site near its 3′ terminus and the nucleic acid segments that contains the ORF of interest also contains an intron/exon splice site near its 5′ terminus. Upon recombination, the nucleic acid segment that encodes the detectable marker is positioned 5′ to the nucleic acid segment that encodes the ORF of interest. Further, these two nucleic acid segments are separated by a recombination site that is flanked by intron/exon splice sites. Excision of the intervening recombination site thus occurs after transcription of the fusion mRNA. Thus, in one aspect, the invention is directed to methods for removing RNA transcribed from recombination sites from transcripts generated from nucleic acids described herein.

Splice sites may introduced into nucleic acid molecules to be used in the present invention in a variety of ways. One method that could be used to introduce intron/exon splice sites into nucleic acid segments is PCR. For example, primers could be used to generate nucleic acid segments corresponding to an ORF of interest and containing both a recombination site and an intron/exon splice site.

The above methods can also be used to remove RNA corresponding to recombination sites when the nucleic acid segment that is recombined with another nucleic acid segment encodes RNA that is not produced in a translatable format. One example of such an instance is where a nucleic acid segment is inserted into a vector in a manner that results in the production of antisense RNA. As discussed below, this antisense RNA may be fused, for example, with RNA that encodes a ribozyme. Thus, the invention also provides methods for removing RNA corresponding to recombination sites from such molecules.

The invention further provides methods for removing amino acid sequences encoded by recombination sites from protein expression products by protein splicing. Nucleotide sequences that encode protein splice sites may be fully or partially embedded in the recombination sites that encode amino acid sequences excised from proteins or protein splice sites may be encoded by adjacent nucleotide sequences. Similarly, one protein splice site may be encoded by a recombination site and another protein splice sites may be encoded by other nucleotide sequences (e.g., nucleic acid sequences of the vector or a nucleic acid of interest).

It has been shown that protein splicing can occur by excision of an intein from a protein molecule and ligation of flanking segments (see, e.g., Derbyshire, et al., Proc. Natl. Acad. Sci. (USA) 95:1356-1357 (1998)). In brief, inteins are amino acid segments that are post-translationally excised from proteins by a self-catalytic splicing process. A considerable number of intein consensus sequences have been identified (see, e.g., Perler, Nucleic Acids Res. 27:346-347 (1999)).

Similar to intron/exon splicing, N- and C-terminal intein motifs have been shown to be involved in protein splicing. Thus, the invention further provides compositions and methods for removing amino acid residues encoded by recombination sites from protein expression products by protein splicing. In particular, this aspect of the invention is related to the positioning of nucleic acid sequences that encode intein splice sites on both the 5′ and 3′ end of recombination sites positioned between two coding regions. Thus, when the protein expression product is incubated under suitable conditions, amino acid residues encoded these recombination sites will be excised.

Protein splicing may be used to remove all or part of the amino acid sequences encoded by recombination sites. Nucleic acid sequence that encode inteins may be fully or partially embedded in recombination sites or may adjacent to such sites. In certain circumstances, it may be desirable to remove considerable numbers of amino acid residues beyond the N- and/or C-terminal ends of amino acid sequences encoded by recombination sites. In such instances, intein coding sequence may be located a distance (e.g., 30, 50, 75, 100, etc. nucleotides) 5′ and/or 3′ to the recombination site.

While conditions suitable for intein excision will vary with the particular intein, as well as the protein that contains this intein, Chong, et al., Gene 192:271-281 (1997), have demonstrated that a modified Saccharomyces cerevisiae intein, referred to as Sce VMA intein, can be induced to undergo self-cleavage by a number of agents including 1,4-dithiothreitol (DTT), β-mercaptoethanol, and cysteine. For example, intein excision/splicing can be induced by incubation in the presence of 30 mM DTT, at 4° C. for 16 hours.

Nucleic Acid Molecules of the Invention

Nucleic acid molecules suitable for use with the present invention include any nucleic acid molecule derived from any source or produced by any method. Such molecules may be derived from natural sources (such as cells (e.g., prokaryotic cells such as bacterial cells, eukaryotic cells such as fungal cells (e.g., yeast cells), plant cells, animals cells (e.g., mammalian cells such as human cells), etc.), viruses, tissues, organs from any animal or non-animal source, and organisms) or may be non-natural (e.g., derivative nucleic acids) or synthetically derived. Such molecules may also include prokaryotic and eukaryotic vectors, plasmids, integration sequences (e.g., transposons), phage or viral vectors, phagemids, cosmids, and the like. The segments or molecules for use in the invention may be produced by any means known to those skilled in the art including, but not limited to, amplification such as by PCR, isolation from natural sources, chemical synthesis, shearing or restriction digest of larger nucleic acid molecules (such as genomic or cDNA), transcription, reverse transcription and the like, and recombination sites may be added to such molecules by any means known to those skilled in the art including ligation of adapters containing recombination sites, attachment with topoisomerases of adapters containing recombination sites, attachment with topoisomerases of adapter primers containing recombination sites, amplification or nucleic acid synthesis using primers containing recombination sites, insertion or integration of nucleic acid molecules (e.g., transposons or integration sequences) containing recombination sites etc.

The nucleic acid molecules of the present invention may be any size, length or conformation. The nucleic acid molecules of the present invention may linear, coiled, closed circles, super-coiled, nicked, single stranded and/or double-stranded. Further, the nucleic acid molecules of the present invention may be DNA or RNA.

In certain embodiments, nucleic acid molecules of the invention may range in size from about 10 bp to about 75 kilobasepairs (kbp), from about 10 bp to about 50 kbp, from about 10 bp to about 40 kbp, from about 10 bp to about 30 kbp, from about 10 bp to about 20 kbp, from about 10 bp to 10 about kbp, from about 10 bp to about 9 kbp, from about 10 bp to about 8 kbp, from about 10 bp to about 7 kbp, from about 10 bp to about 6 kbp, from about 10 bp to about 5 kbp, from about 10 bp to about 2.5 kbp, from about 10 bp to about 1 kbp, from about 10 bp to about 500 bp, from about 10 bp to about 400 bp, from about 10 bp to about 300 bp, from about 10 bp to about 200 bp, from about 10 bp to about 100 bp, from about 10 bp to about 75 bp, from about 10 bp to about 50 bp, from about 10 bp to about 40 bp, from about 10 bp to about 30 bp, from about 10 bp to about 20 bp, from about 10 bp to about 3.

In additional embodiments, nucleic acid molecules of the invention may range in size from about 50 bp to about 75 kilobasepairs (kbp), from about 50 bp to about 50 kbp, from about 50 bp to about 40 kbp, from about 10 bp to about 30 kbp, from about 50 bp to about 20 kbp, from about 50 bp to 10 about kbp, from about 50 bp to about 9 kbp, from about 50 bp to about 8 kbp, from about 50 bp to about 7 kbp, from about 50 bp to about 6 kbp, from about 50 bp to about 5 kbp, from about 50 bp to about 2.5 bp, from about 50 bp to about 1 kbp, from about 50 bp to about 500 bp, from about 50 bp to about 400 bp, from about 50 bp to about 300 bp, from about 50 bp to about 200 bp, from about 50 bp to about 100 bp, from about 50 bp to about 75 bp.

In additional embodiments, nucleic acid molecules of the invention may range in size from about 100 bp to about 75 kilobasepairs (kbp), from about 100 bp to about 50 kbp, from about 100 bp to about 40 kbp, from about 100 bp to about 30 kbp, from about 100 bp to about 20 kbp, from about 100 bp to 10 about kbp, from about 100 bp to about 9 kbp, from about 100 bp to about 8 kbp, from about 100 bp to about 7 kbp, from about 100 bp to about 6 kbp, from about 100 bp to about 5 kbp, from about 100 bp to about 2.5 bp, from about 100 bp to about 1 kbp, from about 100 bp to about 500 bp, from about 100 bp to about 400 bp, from about 100 bp to about 300 bp, or from about 100 bp to about 200 bp.

In additional embodiments, nucleic acid molecules of the invention may range in size from about 5 bp, 10 bp, 15, bp, 20 bp, 25, bp, 30 bp, 40 bp, 50 bp, 60 bp, 70 bp, 75 bp, 80 bp, 90 bp, 100 bp, 125 bp, 150 bp, 175 bp, 200 bp, 250 bp, 300 bp, 350 bp, 400 bp, 450 bp, 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2500 bp, 3000 bp, 4000 bp, 5000 bp, 6000 bp, 7000 bp, 8000 bp, 9000 bp, 10,000 bp, 11,000 bp, 12,000 bp, 13,000 bp, 14,000 bp, 15,000 bp, 16,000 bp, 17,000 bp, 18,000 bp, 19,000 bp, 20,000 bp, 25,000 bp, 30,000 bp, 40,000 bp, 50,000 bp, 60,000 bp, or 75,000 bp in length. In further embodiments, the nucleic acid molecules may comprise at least one segment having one of the above lengths.

In particular embodiments, nucleic acid marker molecules of the invention may range in size from about 5 to about 100 bp, 100 to about 200 bp, 200 to about 300 bp, 400 to about 500 bp, 500 to about 600 bp, 600 to about 700 bp, 700 to about 800 bp, 800 to about 900 bp, 900 to about 1000 bp, 1000 to about 1100 bp, 1100 to about 1200 bp, 1200 to about 1300 bp, 1300 to about 1400 bp, 1400 to about 1500 bp, 1500 to about 1600 bp, 1600 to about 1700 bp, 1700 to about 1800 bp, 1900 to about 2000 bp, 2000 to about 3000 bp, 3000 to about 4000 bp, 4000 to about 5000 bp, 5000 to about 6000 bp, 6000 to about 7000 bp, 7000 to about 8000 bp, 8000 to about 9000 bp, 9000 to about 10,000 bp, 10,000 to about 11,000 bp, 11,000 to about 12,000 bp, 12,000 to about 13,000 bp, 13,000 to about 14,000 bp, 14, 000 to about 15,000 bp, 15,000 to about 16,000 bp, 16,000 to about 17,000 bp, 17,000 to about 18,000 bp, 18,000 to about 19,000 bp, 19,000 to about 20,000 bp, 20,000 to about 25,000 bp, 25,000 to about 30,000 bp, 30,000 to about 40,000 bp, 40,000 to about 50,000 bp, 50,000 to about 60,000 bp, or 60,000 to about 75,000 bp in length.

In other embodiments, nucleic acid marker molecules of the invention may be about 5 bp, 10 bp, 15, bp, 20 bp, 25, bp, 30 bp, 40 bp, 50 bp, 60 bp, 70 bp, 80 bp, 90 bp, 100 bp, 125 bp, 150 bp, 175 bp, 200 bp, 250 bp, 300 bp, 350 bp, 400 bp, 450 bp, 500 bp, 600 bp, 700 bp, 800 bp, 900 bp, 1000 bp, 1100 bp, 1200 bp, 1300 bp, 1400 bp, 1500 bp, 1600 bp, 1700 bp, 1800 bp, 1900 bp, 2000 bp, 2500 bp, 3000 bp, 4000 bp, 5000 bp, 6000 bp, 7000 bp, 8000 bp, 9000 bp, 10,000 bp, 11,000 bp, 12,000 bp, 13,000 bp, 14,000 bp, 15,000 bp, 16,000 bp, 17,000 bp, 18,000 bp, 19,000 bp, 20,000 bp, 30,000 bp, 40,000 bp, 50,000 bp, 60,000 bp, or 75,000 bp in length.

In certain embodiments, nucleic acid molecules of the invention may comprise regions which are recognized by and/or capable of binding proteins.

In further embodiments, these nucleic acid molecules may comprise regions suitable for use as primer sites (e.g., sequences which a primer such as a sequencing primer or amplification primer may hybridize to initiate nucleic acid synthesis, amplification or sequencing), transcription or translation signals or regulatory sequences such as promoters or enhancers, ribosomal binding sites, Kozak sequences, start codons, transcription and/or translation termination signals such as stop codons (which may be optimally suppressed by one or more suppressor tRNA molecules), origins of replication, open reading frame (ORF) sequences and selectable markers.

In another embodiment, nucleic acid molecules of the invention may comprise genes encoding proteins or polypeptides. For example, the nucleic acid molecules of the invention may encode glutathione S-transferase (GST), β-glucuronidase (GUS), histidine tags (HIS6), green fluorescent protein (GFP), yellow fluorescent protein (YFP), cyan fluorescent protein (CFP), blue fluorescent protein (BFP), open reading frame (ORF) sequences, antibody binding domains (e.g. staphylococcal protein A and protein G IgG binding domains), biotin binding domains (e.g. streptavidin, avidin, and domains capable of biotinylation), capture tags (e.g., fluorescein, digoxigenin, FLASH, FLAG, chitin-binding domains), enzymes (e.g. β-galactosidase, β-lactamase, β-glucuronidase (GUS), nucleases, proteases, kinases, phosphatases), a heme group (e.g., cytochrome C, microperoxidase MP-11, horse radish peroxidase) and proteins susceptible to post-translational modification (e.g., phosphorylation (β-casein) and glycosylation).

In a further embodiment, nucleic acid molecules of the invention may comprise genes encoding a binding domain such as complementary determining residues (CDR) of an antibody, a camel antibody, a single-chain antibody, a domain that binds a constant region of an antibody, a domain that binds a nucleic acid (e.g., DNA, RNA, tRNA, ribosomal RNA, mRNA, antisense DNA, a site that regulates gene expression, a nucleic acid from a pathogen, a nucleic acid from a sample, etc.), or a domain that binds a ligand of a receptor. The nucleic acid molecules of the invention may also comprise genes encoding a domain which binds a substance such as a lipid, a small organic molecule, biotin or biotinylated compound, a cell surface receptor or antigen, a crystal or an artificial polymer (e.g., plastic).

In addition, the present invention provides nucleic acid molecules comprising genes encoding segments of highly basic proteins such as histones, or segments of proteins encoded by genes from organisms having highly acidic proteins such as Helobacter, or entirely synthetic nucleic acid molecules encoding segments enriched in basic amino acids such as arginine or lysine, or alternatively, segments enriched in acidic amino acids such as aspartic acid or glutamic acid.

In certain embodiments, nucleic acid molecules of the invention may encode myosin (e.g., H-chain), aprotinin, phosphorylase B, bovine serum albumin (BSA), ovalbumin, carbonic anhydrase, soybean trypsin inhibitor, glutamic dehydrogenase, lactate dehydrogenase, trypsin inhibitor, lysozyme, insulin B chain, insulin A chain, lactate dehydrogenase, RE BPsp98I, β-lactoglobulin, carbonic anhydrase, lysozyme, or fragments or variants thereof.

In a further embodiment, nucleic acid molecules of the invention may encode a protein having a known or predetermined molecular weight and/or known or predetermined pI.

The proteins or fusion proteins encoded by nucleic acid molecules of the invention may be any size and may have any molecular weight and/or pI. In one embodiment, the present invention relates to a protein marker molecule having a molecular weight from about 5 kilodaltons (kDa) to about 500 kDa, from about 5 kDa to about 400 kDa, from about 5 kDa to about 250 kDa, from about 5 kDa to about 125 kDa, from about 5 kDa to about 100 kDa, from about 5 kDa to about 75 kDa, from about 5 kDa to about 50 kDa, from about 5 kDa to about 25 kDa.

In an additional embodiment, the present invention relates to a protein marker molecule having a molecular weight from about 5 kDa to about 10 kDa, from about 10 kDa to about 20 kDa, from about 20 kDa to about 30 kDa, from about 30 kDa to about 40 kDa, from about 40 kDa to about 50 kDa, from about 50 kDa to about 60 kDa, from about 60 kDa to about 80 kDa, from about 80 kDa to about 100 kDa, from about 100 kDa to about 120 kDa, from about 120 kDa to about 200 kDa, from about 200 kDa to about 300 kDa, or from about 300 kDa to about 400 kDa.

In a further embodiment, the present invention relates to a protein marker molecule having a molecular weight of about 20 kDa, about 30 kDa, about 40 kDa, about 50 kDa, about 60 kDa, about 80 kDa, about 100 kDa, about 120 kDa, about 200 kDa, about 300 kDa, or about 400 kDa.

In one embodiment the protein marker molecule may have an isoelectric point (pI) from about 0 to about 14, from about 2 to about 12, from about 2 to about 10, from about 2 to about 9, from about 2 to about 7, from 2 to about 5, from about 3 to about 11, from about 3 to about 10, from about 3 to about 9, from about 3 to about 8, from about 3 to about 7, from about 3 to about 6, from about 3 to about 5, from about 4 to about 10, from about 5 to about 9, from about 5 to about 9, from about 6 to about 8.

In another embodiment the protein marker molecule may have a pI from about 0 to about 12, from about 0 to about 10, from about 0 to about 8, from about 0 to about 6, from about 0 to about 4, from about 0 to about 2, from about 2 to about 14, from about 2 to about 12, from about 2 to about 8, from about 2 to about 6, from about 2 to about 4, from about 4 to about 14, from about 4 to about 12, from about 4 to about 10, from about 4 to about 8, or from about 4 to about 6.

As discussed elsewhere herein, the invention provides a modular system for constructing nucleic acid molecules having a known or predetermined physical characteristic or nucleic acid molecules encoding proteins having a known or predetermined physical characteristic. The methods involve linking at least two nucleic acid molecules, each nucleic acid molecule comprising at least one recombination site. The present invention also includes methods for preparing vectors containing one or more nucleic acid inserts (e.g., 1, 2, 3, 4, 5, 6, 7, 8, 9, 10, 11, 12, 13, 14, 15, 20, 50, etc. inserts).

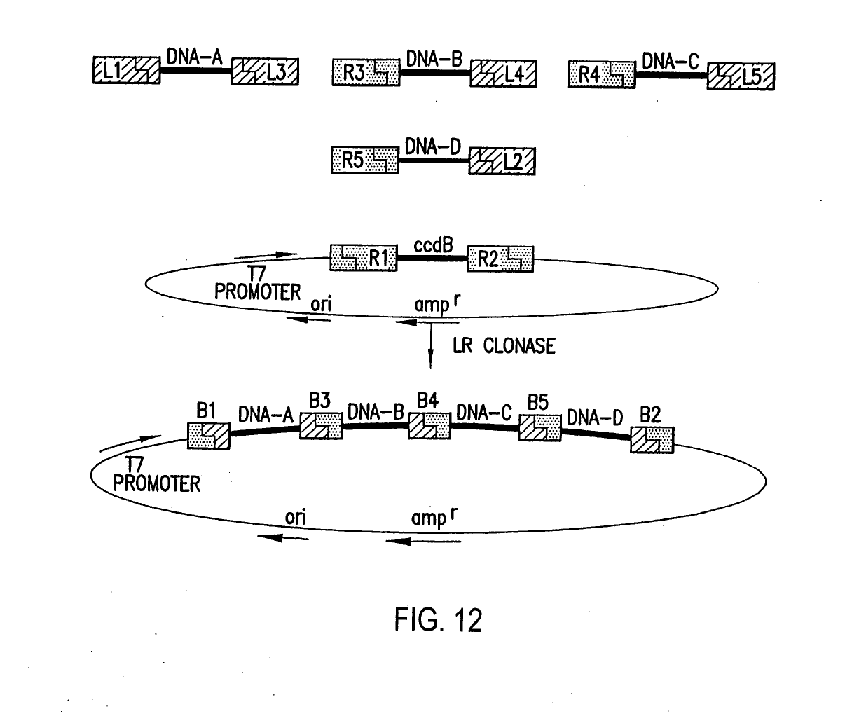

In one general embodiment of the invention, vectors of the invention may be prepared as follows. Nucleic acid molecules which are to ultimately be inserted into the Destination Vector are obtained (e.g., purchased, prepared by PCR or by the preparation of cDNA using reverse transcriptase). Suitable recombination sites are either incorporated into the 5′ and 3′ ends of the nucleic acid molecules during synthesis or added later using, for example, adapter-linkers. When one seeks to prepare a vector containing multiple nucleic acid inserts, these inserts can be inserted into a vector in either one reaction mixture or a series of reaction mixtures. For example, as shown in FIG. 12, multiple nucleic acid segments can be linked end to end and inserted into a vector using reactions performed, for example, in a single reaction mixture. The nucleic acid segments in this reaction mixture can be designed so that recombination sites on their 5′ and 3′ ends result in their insertion into a Destination Vector in a specific order and a specific 5′ to 3′ orientation. Alternatively, nucleic acid segments can be designed so that they are inserted into a Destination Vector without regard to order, orientation (i.e., 5′ to 3′ orientation), the number of inserts, and/or the number of duplicate inserts.

Further, in some instances, one or more of the nucleic acid segments will have a recombination site on only one end. Also, if desired, this end, or these ends, may be linked to other nucleic acid segments by the use of, for example, ligases or topoisomerases. As an example, a linear nucleic acid molecule with an attR1 site on its 5′ terminus can be recombined with a Destination Vector containing a ccdB gene flanked by an attL1 site and an attL2 site. Before, during, or after an LR reaction, the Destination Vector can be cut, for example, by a restriction enzyme on the side of the attR2 site which is opposite to the ccdB gene. Thus, the Destination Vector will be linear after being cut and undergoing recombination. Further, the attR1 site of the nucleic acid molecule will undergo recombination with the attL1 site of the Destination Vector to produce a linear vector which contains the nucleic acid molecule. The resulting linear product can then be circularized using an enzyme such as a ligase or topoisomerases.

Using the embodiment shown in FIG. 12 to exemplify another aspect of the invention, a first DNA segment having an attL1 site at the 5′ end and an attL3 site at the 3′ end is attached by recombination to a second DNA segment having an attR3 site at the 5′ end and an attL4 site at the 3′ end. A third DNA segment having an attR4 site at the 5′ end and an attL5 site at the 3′ end is attached by recombination with the attL4 site on the 3′ end of the second DNA segment. A fourth DNA segment having an attR5 site at the 5′ end and an attL2 site at the 3′ end is attached by recombination with the attL5 site on the 3′ end of the third DNA segment. The Destination Vector contains an attR1 site and an attR2 site which flanks a ccdB gene. Thus, upon reaction with LR CLONASE™, the first, second, third, and fourth DNA segments are inserted into the insertion vector but are flanked or separated by attB1, attB3, attB4, attB5, and attB2 sites.

As one skilled in the art would recognize, multiple variations of the process shown in FIG. 12 are possible. For example, various combinations of attB, attP, attL, and attR sites, as well as other recombination sites, can be used. Similarly, various selection markers, origins of replication, promoters, and other genetic elements can be used. Further, regions which allow for integration into eukaryotic chromosomes (e.g., transposable elements) can be added to these vectors.

As one skilled in the art would recognize, multiple variations of the processes described herein are possible. For example, single use recombination sites can be used to connect individual nucleic acid segments. Thus, eliminating or reducing potential problems associated with arrays of nucleic acid segments engaging in undesired recombination reactions. Further, the processes described above can be used to connect large numbers of individual nucleic acid molecules together in a varying ways. For example, nucleic acid segments can be connected randomly, or in a specified order, both with or without regard to 5′ to 3′ orientation of the segments.

Further, identical copies of one or more nucleic acid segments can be incorporated into another nucleic acid molecule. Thus, the invention also provides nucleic acid molecules which contain multiple copies of a single nucleic acid segment (e.g. nucleic acid segments having the same length in bp or encoding the same protein). Further, the selection of recombination sites positioned at the 5′ and 3′ ends of these segments can be used to determine the exact number of identical nucleic acid segments which are connected and then inserted, for example, into a vector. Such vectors may then be inserted into a host cell where they can, for example, replicate autonomously or integrate into one or more nucleic acid molecules which normally reside in the host cell (e.g., integrate by site-specific recombination or homologous recombination).

As another example, two different nucleic acid segments can be connected using processes of the invention. Recombination sites can be positioned on these segments, for example, such that the segments alternate upon attachment (e.g., Segment A+Segment B+Segment A+Segment B, etc.). In such an instance, “Segment A” can be, for example, a nucleic acid molecule comprising an inducible promoter and “Segment B” can be, for example, a nucleic acid molecule comprising an ORF.

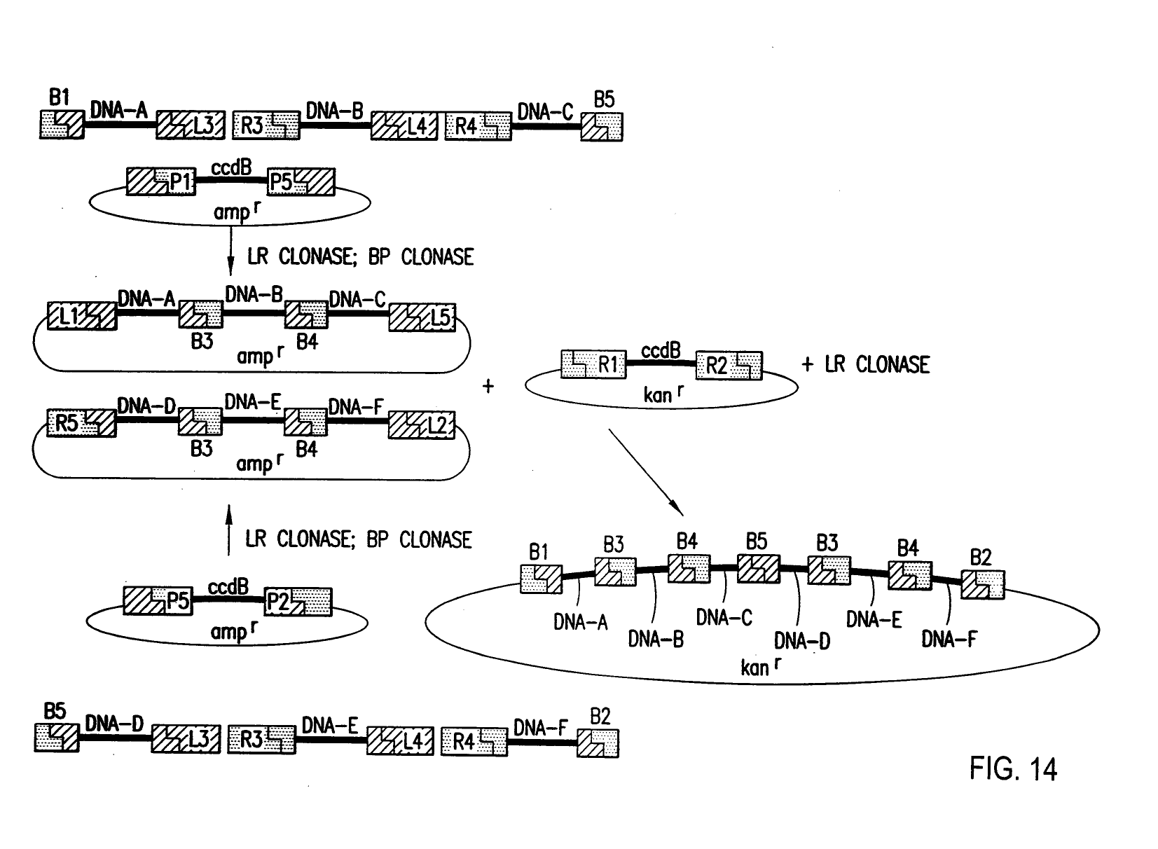

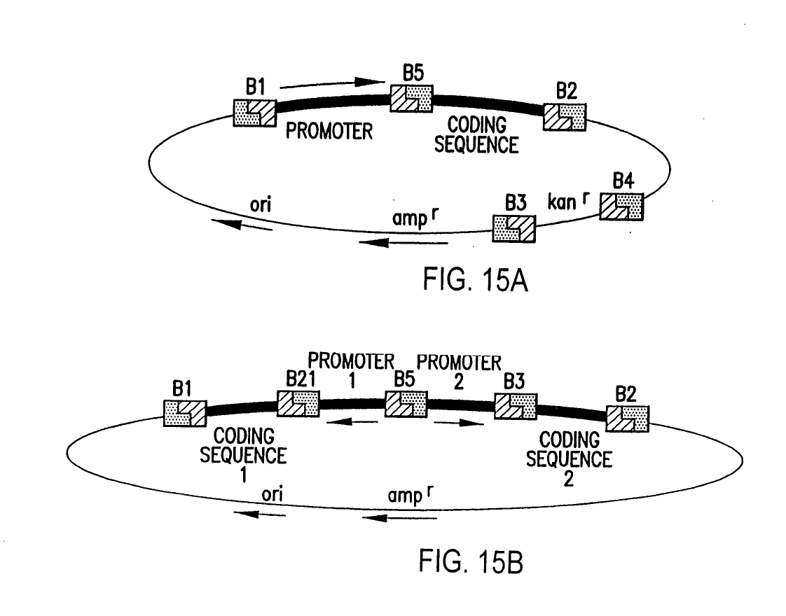

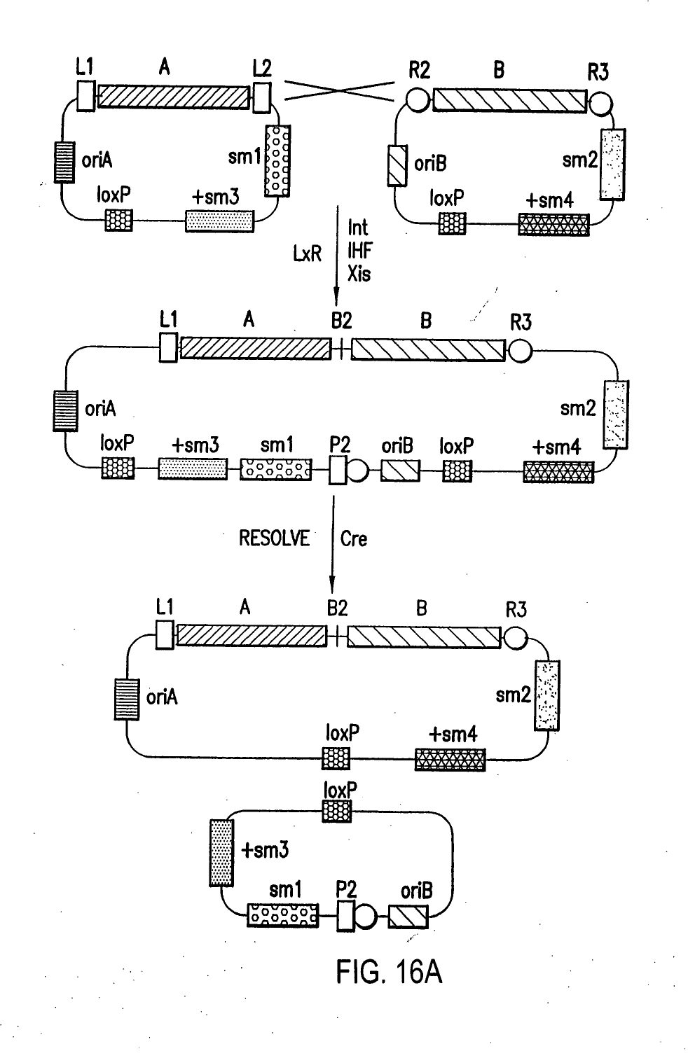

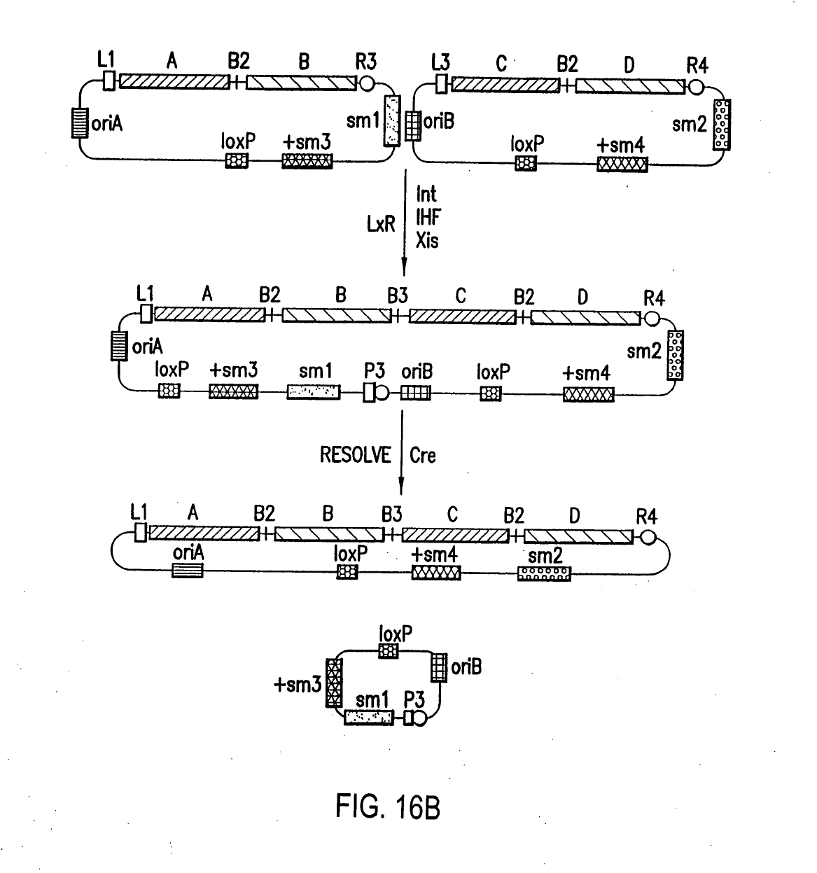

Another example of a multi-step process for inserting multiple DNA segments into a vector is shown in FIG. 14. In this embodiment, three DNA segments are linked to each other in separate recombination reactions and then inserted into separate vectors using LR and BP CLONASE™ reactions. After construction of these two vectors, the inserted DNA segments are transferred to another vector using an LR reaction. This results in all six DNA segments being inserted into a single Destination Vector. As one skilled in the art would recognize, numerous variations of the process shown in FIG. 14 are possible and are included within the scope of the invention.

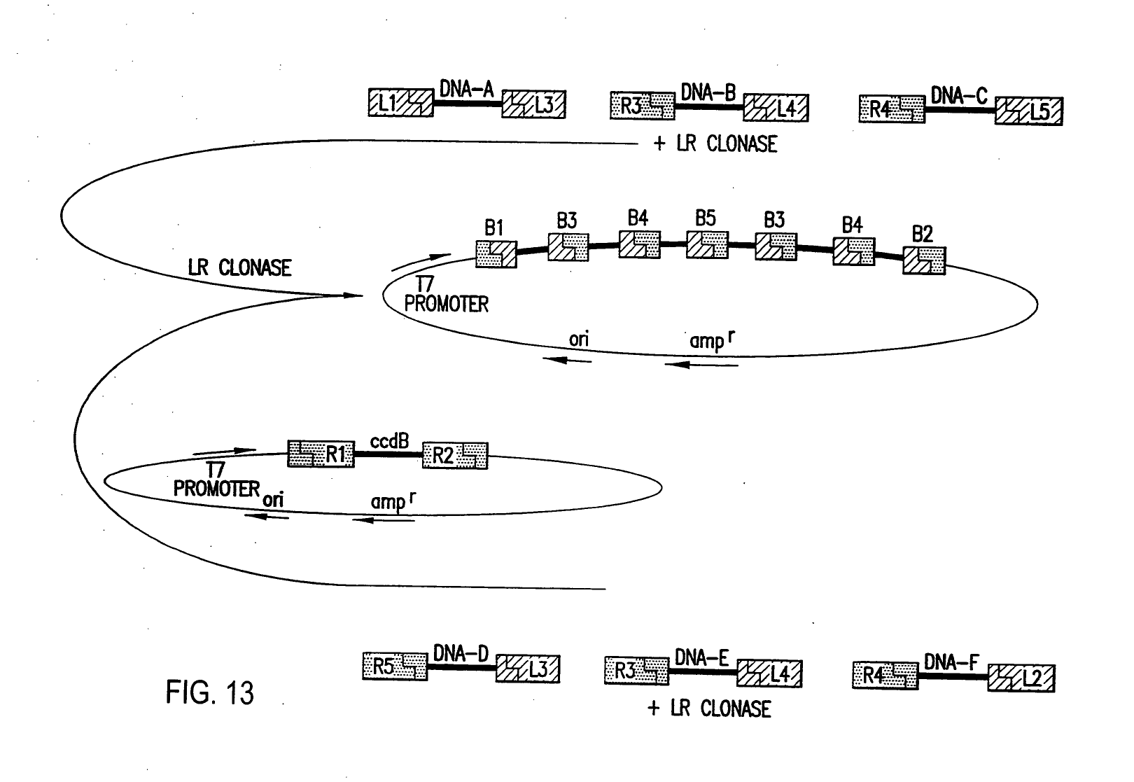

The number of segments which may be connected using methods of the invention in a single step will in general be limited by the number of recombination sites with different specificities which can be used. Further, as described above and represented schematically in FIGS. 13 and 14, recombination sites can be chosen so as to link nucleic acid segments in one reaction and not engage recombination in later reactions. For example, again using the process set out in FIG. 13 for reference, a series of concatamers of ordered nucleic acid segments can be prepared using attL and attR sites and LR Clonase™. These concatamers can then be connected to each other and, optionally, other nucleic acid molecules using another LR reaction. Numerous variations of this process are possible.

Similarly, single use recombination sites may be used to prevent nucleic acid segments, once incorporated into another nucleic acid molecule, from engaging in subsequent recombination reactions. The use of single use recombination sites allows for the production of nucleic acid molecules prepared from an essentially limitless number of individual nucleic acid segments.

Host Cells

Representative host cells that may be used according to the invention include, but are not limited to, bacterial cells, yeast cells, plant cells and animal cells. Bacterial host cells may include Escherichia spp. cells (particularly E. coli cells and most particularly E. coli strains DH10B, Stb12, DH5α, DB3, DB3.1 (preferably E. coli LIBRARY EFFICIENCY™ DB3.1J Competent Cells; Invitrogen Corporation (Carlsbad, Calif.)), DB4 and DB5 (see U.S. application Ser. No. 09/518,188, filed on Mar. 2, 2000, and U.S. Provisional Application No. 60/122,392, filed on Mar. 2, 1999, the disclosures of which are incorporated by reference herein in their entireties), Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Animal host cells may include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly NIH3T3, CHO, COS, VERO, BHK and human cells). Yeast host cells may include Saccharomyces cerevisiae cells and Pichia pastoris cells. These and other suitable host cells are available commercially, for example, from Invitrogen Corporation (Carlsbad, Calif.), American Type Culture Collection (Manassas, Va.), and Agricultural Research Culture Collection (NRRL; Peoria, Ill.).

Methods for introducing the nucleic acid molecules and/or vectors of the invention into the host cells described herein, to produce host cells comprising one or more of the nucleic acid molecules and/or vectors of the invention, will be familiar to those of ordinary skill in the art. For instance, the nucleic acid molecules and/or vectors of the invention may be introduced into host cells using well known techniques of infection, transduction, electroporation, transfection, and transformation. The nucleic acid molecules and/or vectors of the invention may be introduced alone or in conjunction with other the nucleic acid molecules and/or vectors and/or proteins, peptides or RNAs. Alternatively, the nucleic acid molecules and/or vectors of the invention may be introduced into host cells as a precipitate, such as a calcium phosphate precipitate, or in a complex with a lipid. Electroporation also may be used to introduce the nucleic acid molecules and/or vectors of the invention into a host. Likewise, such molecules may be introduced into chemically competent cells such as E. coli. If the vector is a virus, it may be packaged in vitro or introduced into a packaging cell and the packaged virus may be transduced into cells. Thus nucleic acid molecules of the invention may contain and/or encode one or more packaging signal (e.g., viral packaging signals which direct the packaging of viral nucleic acid molecules). Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W.H. Freeman and Co., pp. 213-234 (1992), and Winnacker, E.-L., From Genes to Clones, New York: VCH Publishers (1987), which are illustrative of the many laboratory manuals that detail these techniques and which are incorporated by reference herein in their entireties for their relevant disclosures.

(a) Use of Molecular Markers as Standards

(b) The marker molecules and marker molecule compositions of the present invention may be used as standards in any system commonly used to separate macromolecules, e.g. by size, pI, or other physical or chemical property. The marker molecules and marker molecule compositions may be added to a matrix and exposed to an electromagnetic field which results in movement of the molecular markers through the matrix. Examples of such matrixes include, without limitation, agarose, cross-linked polyacrylamide gels, cross-linked dextran, DEAE-cellulose, DEAE-Sephadex, DEAE Sephacel and the like. The matrices may be in any form or shape, size or porosity. The shapes include slabs, blocks, tubes, columns, membranes and the like. The matrices may contain a number of additives which include, without limitation, denaturant, and buffers. In another embodiment, the marker molecules and marker molecule compositions may be used as markers in capillary electrophoresis. In another embodiment, the marker molecules and marker molecule compositions are used as standards when separating macromolecules by any other method including column chromatography, density gradient centrifugation, ion-exchange chromatography, size exclusion chromatography, thin layer chromatography, liquid chromatography, and the like.

(c) In particular, marker molecules of the present invention may be used in gel electrophoresis systems such as those described below. A considerable number of gel electrophoresis separation systems are known in the art. Further, these systems operate to separate molecules by a variety of properties associated with the molecules being separated. Further, multiple separation principles may be combined to separate molecules (1) in a single gel electrophoresis system or (2) in different gels electrophoresis systems. In other words, molecules may be separated from each other in a one-dimensional gel system which separates molecules based on one or more (e.g., 1, 2, 3, 4, 5, 6, etc.) properties or the same molecules may be separated from each other using a two-dimensional gel, wherein each phase of the separation process separates molecules based on one or more (e.g., 1, 2, 3, 4, 5, 6, etc.) properties. Typically, when a two-dimensional gel system is used, molecules are separated in each of the two dimensions based on at least one different property (e.g., charge in the first dimension and molecular weight in the second dimension). Marker molecules of the present invention may be employed in one-dimensional and two-dimensional gel electrophoresis systems.

(d) As noted above, gel electrophoresis systems may separate molecules based on a variety of properties. Examples of these properties including molecular weight, isoelectric point, and the ability of the molecules to bind detergents (e.g., non-ionic detergents), as well as combinations of these properties. Further, examples of gel electrophoresis systems in which marker molecules of the invention may be employed include polyacrylamide gel electrophoresis (PAGE) (with and without denaturants such as SDS and with and without reducing agents such as dithiothreitol, mercaptoethanol, tris-carboxyethyl-phosphine, tribuytl phoshine and the like), acid-urea gel electrophoresis, acid-urea gel electrophoresis conducted in the presence of one or more detergents (e.g., one or more non-ionic detergent such as TRITON X-100™, sodium deoxycholate, NONIDET P-40™, etc.), and isoelectric focusing. Markers molecules of the invention may be used, for example, with electrophoretic systems such as one-dimensional gel electrophoresis systems, two-dimensional gel electrophoresis systems, capillary electrophoresis systems, and electrokinetic chromatography systems, as well as other gel electrophoresis systems.

(e) In one aspect, the invention includes marker molecules of uniform molecule weight, as well as compositions containing one or more (e.g. 1, 2, 3, 4, 5, 6, 7, 8, 9, 10, 11, 12, 13, 14, 15, 20, 50, etc.) marker molecules which differ in molecular weight. These marker molecules are particularly suited for use with gel electrophoresis systems which separate molecules on the basis of molecular weight. Examples of gel electrophoresis systems which separate molecules mainly on the basis of molecular weight include SDS-PAGE systems (Laemmli, U. K., Nature 227:680-685 (1970)).

(f) In another aspect, the invention includes marker molecules of uniform isoelectric point, as well as compositions containing one or more (e.g., 1, 2, 3, 4, 5, 6, 7, 8, 9, 10, 11, 12, 13, 14, 15, 20, 50, etc.) marker molecules which differ in isoelectric point. These marker molecules are particularly suited for use with gel electrophoresis systems which separate molecules on the basis of isoelectric point (e.g., isoelectric focusing systems).

(g) It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof.

(h) Kits

In another aspect, the present invention provides kits comprising one or more of the marker molecules or compositions described. Kits serve to expedite the performance of, for example, methods of the invention by providing multiple components and reagents packed together. Further, reagents of these kits can be supplied in pre-measured units so as to increase precision and reliability of the methods. Kits of the present invention will generally comprise a carton such as a box; one or more containers such as boxes, tubes, ampoules, jars, or bags; one or more (e.g., 1, 2, 3, etc.) pre-cast gels and the like; one or more (e.g., 1, 2, 3, etc.) buffers; and instructions for use of kit components.

The present invention further relates to instructions for performing one or more methods of the invention (e.g., preparing molecular markers). Such instructions can instruct a user of conditions suitable for performing methods of the invention. Instructions of the invention can be in a tangible form, for example, written instructions (e.g., typed on paper), or can be in an intangible form, for example, accessible via a computer (e.g., over the internet). Also provided is an instruction set that provides, in part, directions for performing one or more method of the invention. Such an instruction set can instruct a user of conditions suitable for, for example, preparing molecular markers. Thus, the invention include instructions and instructions sets for performing one or more methods of the invention, as well as methods for performing methods of the invention by following such instructions.

In various aspects, a kit of the invention can contain one or more (e.g., 1, 2, 3, 4, 5, 6, 7, etc.) of the following components: (1) one or more sets of instructions, including, for example, instructions for performing methods of the invention; (2) one or more cells, including, for example, one or more prokaryotic (e.g., bacterial) cells; one or more insect cells; one or more mammalian cells, for example, cells that are adapted for growth in a tissue culture medium, (3) one or more topoisomerases, including, for example, one or more type IA, type IB, or type II topoisomerases, or combinations thereof; (4) one or more nucleic acid molecules, including, for example, one or more vectors, which can be cloning vector or expression vector, one or more transcriptional or translational regulatory elements (e.g., a Shine-Delgarno sequence, a ribosome binding site, a transcriptional promoter and/or enhancer, or a polyadenylation site), any or all of which can be bound to one or more topoisomerases), or one or more coding sequences (e.g., a nucleotide sequence encoding a reporter molecule, detectable transcription or translation product, affinity tag, etc.); (5) one or more cartons, boxes and/or containers for storing and/or transporting kit components (e.g., a box in which to ship components, or a plastic vial in which to store dry, liquid or lyophilized reagents or other kit materials); (6) one or more container containing water (e.g., distilled water) or other aqueous or liquid material; (7) one or more containers containing one or more buffers, which can be buffers in dry, powder form or reconstituted in a liquid such as water, including in a concentrated form such as 2×, 3×, 4×, 5×, etc.); and/or (8) one or more containers containing one or more salts (e.g., sodium chloride, potassium chloride, magnesium chloride, which can be in a dry, powder form or reconstituted in a liquid such as water).

A kit of the invention can include an instruction set, or the instructions can be provided independently of a kit. Such instructions are characterized, in part, in that they provide a user with information related to preparing and/or using a molecular marker molecule. Instructions can be provided in a kit, for example, written on paper or in a computer readable form provided with the kit, or can be made accessible to a user via the internet, for example, on the world wide web at a URL (uniform resources link; i.e., “address”) specified by the provider of the kit or an agent of the provider. Such instructions direct a user of the kit or other party of particular tasks to be performed or of particular ways for performing a task. In one aspect, the instructions can, for example, instruct a user of the kit as to reaction and/or culture conditions, including, for example, buffers, temperature, and time, to prepare and/or use a molecular marker.

The present invention also provides instructions for performing methods of the invention, such as instructions for a method of preparing a nucleic acid marker molecule comprising providing two or more starting nucleic acid molecules comprising a nucleic acid segment having a known or predetermined physical characteristic, each segment flanked by at least one recombination site, wherein said recombination sites are chosen such that at least one recombination site is capable of recombining with at least one recombination site of another starting nucleic acid molecule, and contacting said starting nucleic acid molecules under conditions causing recombination between the recombination sites, thereby producing a product nucleic acid molecule; and isolating said product nucleic acid molecule.

Kits of the invention may also provide instructions and/or materials for practicing a method of preparing a nucleic acid marker molecule comprising providing n starting nucleic acid molecules comprising a nucleic acid segment L, each segment flanked by at least one recombination site, wherein said recombination sites are chosen such that at least one recombination site is capable of recombining with at least one recombination site of another starting nucleic acid molecule, and wherein L is a segment consisting of a known or predetermined number of bp, contacting said starting nucleic acid molecules under conditions causing recombination between the recombination sites, thereby producing a product nucleic acid molecule comprising (L)_{n,}, wherein n is any integer greater than 2, and isolating said product nucleic acid molecule.

Kits of the invention may also provide instructions and/or materials for practicing a method of preparing a nucleic acid marker molecule comprising providing one or more starting nucleic acid molecules comprising a nucleic acid segment B, wherein said segment B is flanked by at least one recombination site capable of recombining with at least one recombination site of another starting nucleic acid molecule, providing n starting nucleic acid molecules comprising a nucleic acid segment L, each segment flanked by at least one recombination site, wherein said recombination sites are chosen such that at least one recombination site is capable of recombining with at least one recombination site of another starting nucleic acid molecule and wherein L is a segment consisting of a known or predetermined number of bp, contacting said starting nucleic acid molecules under conditions causing recombination between the recombination sites, thereby joining end to end said segments and producing a product nucleic acid molecule comprising B-(L)ₙ, and isolating said product nucleic acid molecule.

Kits of the invention may comprise instructions and/or materials for practicing a method of preparing a protein marker molecule comprising providing at least two starting nucleic acid molecules, wherein at least one nucleic acid molecule comprises a segment encoding a protein having a known or predetermined physical characteristic and wherein each nucleic acid molecule comprises at least one recombination site capable of recombining with a recombination site present on another segment or starting nucleic acid, contacting said starting nucleic acid molecules under conditions causing recombination between the recombination sites, thereby joining said nucleic acid molecules, or said segments, and producing a product nucleic acid molecule, transforming said product nucleic acid molecule into a host cell, causing the product nucleic molecule of (c) to express the encoded protein, and purifying said expressed protein. Kits of the invention may comprise instructions for practicing a method of preparing a protein marker molecule comprising providing n starting nucleic acid molecules comprising a nucleic acid segment encoding M, each segment flanked by at least one recombination site, and wherein said recombination sites are chosen such that at least one recombination site is capable of recombining with at least one recombination site of another starting nucleic acid molecule, contacting said starting nucleic acid molecules under conditions causing recombination between the recombination sites, thereby joining end to end said segments and producing a product nucleic acid molecule which encodes a fusion protein comprising (M)ₙ, transforming said product nucleic acid molecule into a host cell, causing the nucleic acid molecule of (c) to express the encoded protein, and purifying said expressed proteins.

Kits of the invention may comprise instructions and/or materials for practicing a method of preparing a protein marker molecule comprising providing one or more starting nucleic acid molecules comprising a nucleic acid segment encoding E, wherein said segment is flanked by at least one recombination site capable of recombining with at least one recombination site of another starting nucleic acid molecule, providing n starting nucleic acid molecules comprising a nucleic acid segment encoding M, each segment flanked by at least one recombination site, and wherein said recombination sites are chosen such that at least one recombination site is capable of recombining with at least one recombination site of another starting nucleic acid molecule, contacting said starting nucleic acid molecules under conditions causing recombination between the recombination sites, thereby joining end to end said segments and producing a product nucleic acid molecule encoding a protein comprising E-(M)ₙ, transforming said product nucleic acid molecule into a host cell, causing the nucleic acid molecule of (c) to express the encoded protein, and purifying said expressed proteins.

Such instructions can further include directions for providing conditions such as buffer and salt conditions, as well as temperature and time for performing reactions of the invention as described, for example, elsewhere herein. The instructions of the invention can be in a tangible form, for example, printed or otherwise imprinted on paper, or in an intangible form, for example, present on an internet web page at a defined and accessible URL).

It will be recognized that a full text of instructions for performing a method of the invention or, where the instructions are included with a kit, for using the kit, need not be provided. One example of a situation in which a kit of the invention, for example, would not contain such full length instructions is where the provided directions inform a user of the kits where to obtain instructions for practicing methods for which the kit can be used. Thus, instructions for performing methods of the invention can be obtained from internet web pages, separately sold or distributed manuals or other product literature, etc. The invention thus includes kits that direct a kit user to one or more locations where instructions not directly packaged and/or distributed with the kits can be found. Such instructions can be in any form including, but not limited to, electronic or printed forms.

Business Methods

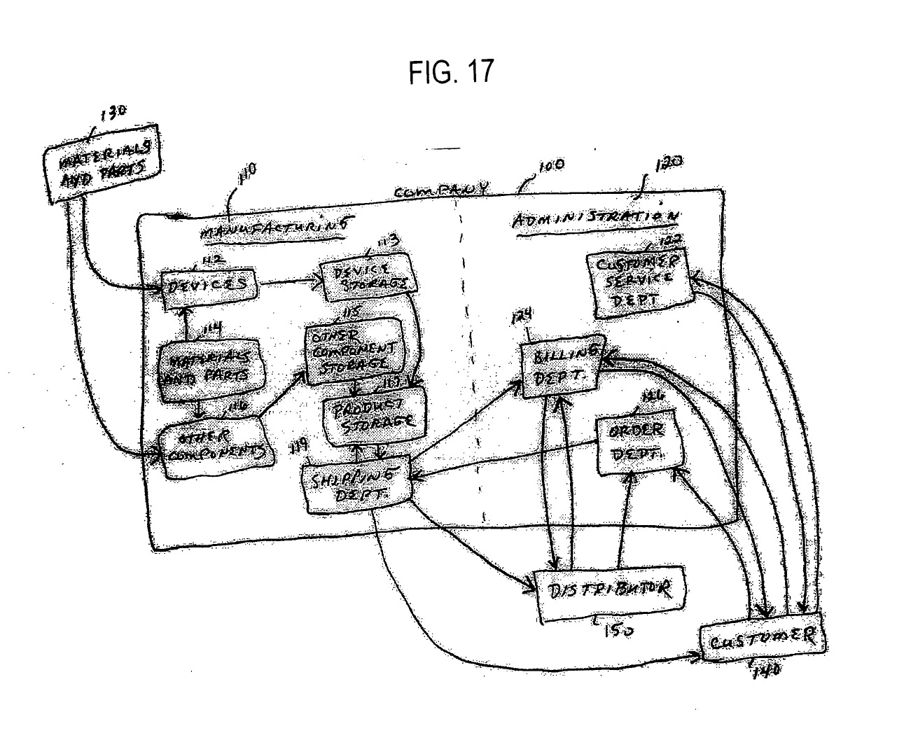

The present invention also provides a system and method of providing company products to a party outside of the company, for example, a system and method for providing a customer or a product distributor a product of the company such as a kit containing materials for the preparation and use of molecular markers and/or instructions for such preparation and use. FIG. 17 provides a schematic diagram of a product management system. In practice, the blocks in FIG. 17 can represent an intra-company organization, which may include departments in a single building or in different buildings, a computer program or suite of programs maintained by one or more computers, a group of employees, a computer I/O device such as a printer or fax machine, a third party entity or company that is otherwise unaffiliated with the company, or the like.

The product management system as shown in FIG. 17 is exemplified by company 100, which receives input in the form of an order from a party outside of the company, e.g., distributor 150 or customer 140, to order department 126, or in the form of materials and parts 130 from a party outside of the company; and provides output in the form of a product delivered from shipping department 119 to distributor 150 or customer 140. Company 100 system is organized to optimize receipt of orders and delivery of a products to a party outside of the company in a cost efficient manner, particularly instructions or a kit of the present invention, and to obtain payment for such product from the party in a timely manner.

With respect to the methods of the present invention, the term “materials and parts” refers to items that are used to make a device, other component, or product, which generally is a device, other component, or product that company sells to a party outside of the company. As such, materials and parts include, for example, topoisomerases, recombinases, starting nucleic acid molecules with one or more recombination sites, nucleic acid segments, nucleotides, host cells, polymerases, amino acids, culture media, buffers, paper, ink, reaction vessels, etc. In comparison, the term “devices”, “other components”, and “products” refer to items sold by the company. Devices are exemplified by nucleic acid marker molecules or protein marker molecules that are to be sold by the company. Other components are exemplified by instructions, including instructions for the preparation and use of molecular markers. Other components also can be items that may be included in a kit, e.g., a kit product, for example, reagents for manipulating nucleic acid molecules (e.g., performing recombinational cloning and/or topoisomerase mediated joining). Kits may include multiple marker molecules and instructions for use. Reagents may include, for example, buffers, salts, cofactors and the like. Other components may include host cells. As such, it will be recognized that an item useful as materials and parts as defined herein further can be considered an other component, which can be sold by the company. The term “commercial products” refers to devices, other components, or combinations thereof, including combinations with additional materials and parts, that are sold or desired to be sold or otherwise provided by a company to one or more parties outside of the company. Commercial products are exemplified herein by kits, which can contain instructions according to the present invention, and one or more nucleic acid molecules and/or protein marker molecules, which may be comprise one or more recombination sites, reagents, or combinations thereof.

Referring to FIG. 17, company 100 includes manufacturing 110 and administration 120. Devices 112 and other components 114 are produced in manufacturing 110, and can be stored separately therein such as in device storage 113 and other component storage 115, respectively, or can be further assembled and stored in product storage 117. Materials and parts 130 can be provided to company 100 from an outside source and/or materials and parts 114 can be prepared in company, and used to produce devices 112 and other components 116, which, in turn, can be assembled and sold as a product. Manufacturing 110 also includes shipping department 119, which, upon receiving input as to an order, can obtain products to be shipped from product storage 117 and forward the product to a party outside the company.

For purposes of the present invention, product storage 117 can be used to store instructions for preparation and use of molecular markers; or can store kits, which can contain at least one nucleic acid marker molecule and/or protein marker molecule, and, optionally, instructions as disclosed herein; or can store a combination of such instructions and/or kits (e.g., collections of more than one marker molecule packaged for shipment to customers). Upon receiving input from order department 126, for example, that customer 140 has ordered such a kit and instructions, shipping department 119 can obtain from product storage 117 such kit for shipping, and can further obtain such instructions in a written form to include with the kit, and ship the kit and instructions to customer 140 (and providing input to billing department 124 that the product was shipped; or shipping department 119 can obtain from product storage 117 the kit for shipping, and can further provide the instructions to customer 140 in an electronic form, by accessing a database in company 100 that contains the instructions, and transmitting the instructions to customer 140 via the internet (not shown).

As further exemplified in FIG. 17, administration 120 includes order department 126, which receives input in the form of an order for a product from customer 140 or distributor 150. Order department 126 then provides output in the form of instructions to shipping department 119 to fill the order, i.e., to forward products as requested to customer 140 or distributor 150. Shipping department 119, in addition to filling the order, further provides input to billing department 124 in the form of confirmation of the products that have been shipped. Billing department 124 then can provide output in the form of a bill to customer 140 or distributor 150 as appropriate, and can further receive input that the bill has been paid, or, if no such input is received, can further provide output to customer 140 or distributor 150 that such payment may be delinquent.

Additional optional component of company 100 include customer service department 122, which can receive input from customer 140 and can provide output in the form of feedback or information to customer 140. Furthermore, although not shown in FIG. 17, customer service 122 can receive input or provide output to any other component of company. For example, customer service department 122 can receive input from customer 140 indicating that an ordered product was not received, wherein customer service department 122 can provide output to shipping department 119 and/or order department 126 and/or billing department 124 regarding the missing product, thus providing a means to assure customer 140 satisfaction. Customer service department 122 also can receive input from customer 140 in the form of requested technical information, for example, for confirming that instructions of the invention can be applied to the particular need of customer 140, and can provide output to customer 140 in the form of a response to the requested technical information.

As such, the components of company 100 are suitably configured to communicate with each other to facilitate the transfer of materials and parts, devices, other components, products, and information within company 100, and company 100 is further suitably configured to receive input from or provide output to an outside party. For example, a physical path can be utilized to transfer products from product storage 117 to shipping department 119 upon receiving suitable input from order department 126. Order department 126, in comparison, can be linked electronically with other components within company 100, for example, by a communication network such as an intranet, and can be further configured to receive input, for example, from customer 140 by a telephone network, by mail or other carrier service, or via the internet. For electronic input and/or output, a direct electronic link such as a T1 line or a direct wireless connection also can be established, particularly within company 100 and, if desired, with distributor 150 or materials or parts 130 provider, or the like.

Although not illustrated, company 100 may comprise one or more data collection systems, including, for example, a customer data collection system, which can be realized as a personal computer, a computer network, a personal digital assistant (PDA), an audio recording medium, a document in which written entries are made, any suitable device capable of receiving data, or any combination of the foregoing. Data collection systems can be used to gather data associated with a customer 140 or distributor 150, including, for example, a customer's shipping address and billing address, as well as more specific information such as the customer's ordering history and payment history, such data being useful, for example, to determine that a customer has made sufficient purchases to qualify for a discount on one or more future purchases.

Company 100 can utilize a number of software applications to provide components of company 100 with information or to provide a party outside of company access to one or more components of company 100, for example, access to order department 126 or customer service department 122. Such software applications can comprise a communication network such as the Internet, a local area network, or an intranet. For example, in an internet-based application, customer 140 can access a suitable web site and/or a web server that cooperates with order department 126 such that customer 140 can provide input in the form of an order to order department 126. In response, order department 126 can communicate with customer 140 to confirm that the order has been received, and can further communicate with shipping department 119, providing input that products such as a kit of the invention, which contains, for example, a topoisomerase charged nucleic acid molecule and instructions for use, should be shipped to customer 140. In this manner, the business of company 100 can proceed in an efficient manner.

In a networked arrangement, billing department 124 and shipping department 119, for example, can communicate with one another by way of respective computer systems. As used herein, the term “computer system” refers to general purpose computer systems such as network servers, laptop systems, desktop systems, handheld systems, personal digital assistants, computing kiosks, and the like. Similarly, in accordance with known techniques, distributor 150 can access a web site maintained by company 100 after establishing an online connection to the network, particularly to order department 126, and can provide input in the form of an order. If desired, a hard copy of an order placed with order department 126 can be printed from the web browser application resident at distributor 150.

The various software modules associated with the implementation of the present invention can be suitably loaded into the computer systems resident at company 100 and any party outside of company 100 as desired, or the software code can be stored on a computer-readable medium such as a floppy disk, magnetic tape, or an optical disk. In an online implementation, a server and web site maintained by company 100 can be configured to provide software downloads to remote users such as distributor 150, materials and parts 130, and the like. When implemented in software, the techniques of the present invention are carried out by code segments and instructions associated with the various process tasks described herein.

Accordingly, the present invention further includes methods for providing various aspects of a product (e.g., a kit and/or instructions of the invention), as well as information regarding various aspects of the invention, to parties such as the parties shown as customer 140 and distributor 150 in FIG. 17. Thus, methods for selling devices, products and methods of the invention to such parties are provided, as are methods related to those sales, including customer support, billing, product inventory management within the company, etc. Examples of such methods are shown in FIG. 17, including, for example, wherein materials and parts 130 can be acquired from a source outside of company 100 (e.g., a supplier) and used to prepare devices (e.g., nucleic acid and/or protein marker molecules) used in preparing a composition or practicing a method of the invention, for example, kits, which can be maintained as an inventory in product storage 117. It should be recognized that devices 112 can be sold directly to a customer and/or distributor (not shown), or can be combined with one or more other components 116, and sold to a customer and/or distributor as the combined product. The other components 116 can be obtained from a source outside of company 100 (materials and parts 130) or can be prepared within company 100 (materials and parts 114). As such, the term “commercial product” is used generally herein to refer an item sent to a party outside of the company (a customer, a distributor, etc.) and includes items such as devices 112, which can be sent to a party alone or as a component of a kit or the like.

At the appropriate time, the product is removed from product storage 117, for example, by shipping department 119, and sent to a requesting party such as customer 140 or distributor 150. Typically, such shipping occurs in response to the party placing an order, which is then forwarded the within the organization as exemplified in FIG. 17, and results in the ordered product being sent to the party. Data regarding shipment of the product to the party is transmitted further within the organization, for example, from shipping department 119 to billing department 124, which, in turn, can transmit a bill to the party, either with the product, or at a time after the product has been sent. Further, a bill can be sent in instances where the party has not paid for the product shipped within a certain period of time (e.g., within 30 days, within 45 days, within 60 days, within 90 days, within 120 days, within from 30 days to 120 days, within from 45 days to 120 days, within from 60 days to 120 days, within from 90 days to 120 days, within from 30 days to 90 days, within from 30 days to 60 days, within from 30 days to 45 days, within from 60 days to 90 days, etc.). Typically, billing department 124 also is responsible for processing payment(s) made by the party. It will be recognized that variations from the exemplified method can be utilized; for example, customer service department 122 can receive an order from the party, and transmit the order to shipping department 119 (not shown), thus serving the functions exemplified in FIG. 17 by order department 126 and the customer service department 122.

The methods of the invention also include providing technical service to parties using a product, particularly a kit of the invention. While such a function can be performed by individuals involved in product research and development, inquiries related to technical service generally are handled, routed, and/or directed by an administrative department of the organization (e.g., customer service department 122). Often communications related to technical service (e.g., solving problems related to use of the product or individual components of the product) require a two way exchange of information, as exemplified by arrows indicating pathways of communication between customer 150 and customer service department 122.

As mentioned above, any number of variations of the process exemplified in FIG. 17 are possible and within the scope of the invention. Accordingly, the invention includes methods (e.g., business methods) that involve (1) the production of products (e.g., nucleic acid and/or protein molecules, kits that contain instructions for performing methods of the invention, etc.); (2) receiving orders for these products; (3) sending the products to parties placing such orders; (4) sending bills to parties obliged to pay for products sent to such; and/or (5) receiving payment for products sent to parties. For example, methods are provided that comprise two or more of the following steps: (a) obtaining parts, materials, and/or components from a supplier; (b) preparing one or more first products (e.g., one or more nucleic acid and/or protein marker molecules); (c) storing the one or more first products of step (b); (d) combining the one or more first products of step (b) with one or more other components to form one or more second products (e.g., a kit); (e) storing the one or more first products of step (b) or one or more second products of step (d); (f) obtaining an order a first product of step (b) or a second product of step (d); (g) shipping either the first product of step (b) or the second product of step (d) to the party that placed the order of step (f); (h) tracking data regarding to the amount of money owed by the party to which the product is shipped in step (g); (i) sending a bill to the party to which the product is shipped in step (g); (i) obtaining payment for the product shipped in step (g) (generally, but not necessarily, the payment is made by the party to which the product was shipped in step (g); and (k) exchanging technical information between the organization and a party in possession of a product shipped in step (d) (typically, the party to which the product was shipped in step (g)).

The present invention also provides a system and method for providing information as to availability of a product (e.g., a device product, a kit product, and the like) to parties having potential interest in the availability of the kit product. Such a method of the invention, which encompasses a method of advertising to the general or a specified public, the availability of the product, particularly a product comprising instructions and/or a kit of the present invention, can be performed, for example, by transmitting product description data to an output source, for example, an advertiser; further transmitting to the output source instructions to publish the product information data in media accessible to the potential interested parties; and detecting publication of the data in the media, thereby providing information as to availability of the product to parties having potential interest in the availability of the product.

Accordingly, the present invention provides methods for advertising and/or marketing devices, products, and/or methods of the invention, such methods providing the advantage of inducing and/or increasing the sales of such devices, products, and/or methods. For example, advertising and/or marketing methods of the invention include those in which technical specifications and/or descriptions of devices and/or products; methods of using the devices and/or products; and/or instructions for practicing the methods and/or using the devices and/or products are presented to potential interested parties, particularly potential purchasers of the product such as customers, distributors, and the like. In particular embodiments, the advertising and/or marketing methods involve presenting such information in a tangible form or in an intangible to the potential interested parties. As disclosed herein and well known in the art, the term “intangible form” means a form that cannot be physically handled and includes, for example, electronic media (e.g., e-mail, internet web pages, etc.), broadcasts (e.g., television, radio, etc.), and direct contacts (e.g., telephone calls between individuals, between automated machines and individuals, between machines, etc.); whereas the term “tangible form” means a form that can be physically handled.

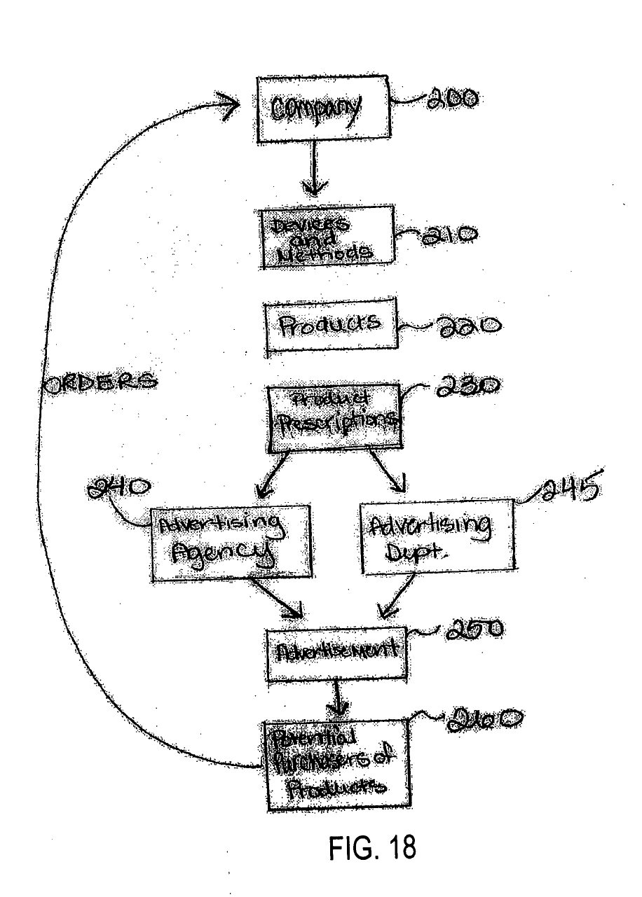

FIG. 18 provides a schematic diagram of an information providing management system as encompassed within the present invention. In practice, the blocks in FIG. 18 can represent an intra-company organization, which can include departments in a single building or in different buildings, a computer program or suite of programs maintained by one or more computers, a group of employees, a computer I/O device such as a printer or fax machine, a third party entity or company that is otherwise unaffiliated with the company, or the like.

The information providing management system as shown in FIG. 18 is exemplified by company 200, which makes, purchases, or otherwise makes available devices and methods 210 that alone, or in combination, provide products 220, for example, instructions, devices and/or kits of the present invention, that company 200 wishes to sell to interested parties. To this end, product descriptions 230 are made, providing information that would lead potential users to believe that products 220 can be useful to user. In order to effect transfer of product descriptions 230 to the potential users, product descriptions 230 is provided to advertising agency 240, which can be an entity separate from company 200, or to advertising department 260, which can be an entity related to company 200, for example, a subsidiary. Based on the product descriptions 230, advertisement 250 is generated and is provided to media accessible to potential purchasers of products 260, whom may then contact company 200 to purchase products 220.

By way of example, product descriptions 230 can be in a tangible form such as written descriptions, which can be delivered (e.g., mailed, sent by courier, etc) to advertising agency 240 and/or advertising department 250, or can be in an intangible form such as entered into and stored in a database (e.g., on a computer, in an electronic media, etc.) and transmitted to advertising agency 240 and/or advertising department 250 over a telephone line, T1 line, wireless network, or the like. Similarly, advertisement 250 can be a tangible or intangible form such that it conveniently and effectively can be provided to potential parties of interest (e.g., potential purchasers of product 260). For example, advertisement 250 can be provided in printed form as flyers (e.g., at a meeting or other congregation of potential interested parties) or as printed pages (or portions thereof) in magazines known to be read by the potential interested parties (e.g., trade magazines, journals, newspapers, etc.). In addition, or alternatively, advertisement 250 can be provided in the form of directed mailing of computer media containing the advertisement (e.g., CDs, DVDs, floppy discs, etc.) or of e-mail (i.e., mail or e-mail that is sent only to selected parties, for example, parties known to members of an organization that includes or is likely to include potential users of products 220); of web pages (e.g., on a website provided by company 200, or having links to the company 200 website); or of pop-up or pop-under ads on web pages known to be visited by potential purchaser of products 260, and the like. Potential purchasers of products 260, upon being apprised of the availability of the products 220, for example, the kits of the present invention, then can contact company 200 and, if so desired, can order said products 220 for company 200 (see FIG. 17).

(a) Having now described the present invention in detail, the same will be more clearly understood by reference to the following examples, which are included herewith for purposes of illustration only and are not intended to be limiting of the invention.

The entire disclosures of U.S. application Ser. No. 08/486,139 (now abandoned), filed Jun. 7, 1995, U.S. application Ser. No. 08/663,002, filed Jun. 7, 1996 (now U.S. Pat. No. 5,888,732), U.S. application Ser. No. 09/233,492, filed Jan. 20, 1999, (now U.S. Pat. No. 6,143,557), U.S. application Ser. No. 09/005,476, filed Jan. 12, 1998 (now U.S. Pat. No. 6,171,861), U.S. application Ser. No. 09/233,492, filed Jan. 20, 1999 (now U.S. Pat. No. 6,270,969), U.S. Appl. No. 60/065,930, filed Oct. 24, 1997, U.S. application Ser. No. 09/177,387 filed Oct. 23, 1998, U.S. application Ser. No. 09/296,280, filed Apr. 22, 1999, (now U.S. Pat. No. 6,277,608), U.S. application Ser. No. 09/296,281, filed Apr. 22, 1999, (now abandoned), U.S. Appl. No. 60/108,324, filed Nov. 13, 1998, U.S. application Ser. No. 09/438,358, filed Nov. 12, 1999, U.S. application Ser. No. 09/695,065, filed Oct. 25, 2000, U.S. application Ser. No. 09/432,085 filed Nov. 2, 1999, U.S. Appl. No. 60/122,389, filed Mar. 2, 1999, U.S. Appl. No. 60/126,049, filed Mar. 23, 1999, U.S. Appl. No. 60/136,744, filed May 28, 1999, U.S. Appl. No. 60/122,392, filed Mar. 2, 1999, U.S. Appl. No. 60/161,403, filed Oct. 25, 1999, U.S. Appl. No. 60/169,983, filed Dec. 10, 1999, U.S. Appl. No. 60/188,000, filed Mar. 9, 2000, U.S. application Ser. No. 09/732,914, filed Dec. 11, 2000, (published as 20020007051 A1), and U.S. application Ser. No. 09/517,466, filed Mar. 2, 2000, U.S. Appl. No. 60/402,920, filed Aug. 14, 2002, are herein incorporated by reference.

### EXAMPLES

### Example 1

### Simultaneous Cloning of Two Nucleic Acid Segments Using an LR Reaction

Two nucleic acid segments may be cloned in a single reaction using methods of the present invention. Methods of the present invention may comprise providing a first nucleic acid segment flanked by a first and a second recombination site, providing a second nucleic acid segment flanked by a third and a fourth recombination site, wherein either the first or the second recombination site is capable of recombining with either the third or the fourth recombination site, conducting a recombination reaction such that the two nucleic acid segments are recombined into a single nucleic acid molecule and cloning the single nucleic acid molecule.

With reference to FIG. 2, two nucleic acid segments flanked by recombination sites may be provided. Those skilled in the art will appreciate that the nucleic acid segments may be provided either as discrete fragments or as part of a larger nucleic acid molecule and may be circular and optionally super-coiled or linear. The sites can be selected such that one member of a reactive pair of sites flanks each of the two segments.

By “reactive pair of sites,” what is meant is two recombination sites that can, in the presence of the appropriate enzymes and cofactors, recombine. For example, in some preferred embodiments, one nucleic acid molecule may comprise an attR site while the other comprises an attL site that reacts with the attR site. As the products of an LR reaction are two molecules, one of which comprises an attB site and one of which comprises an attP site, it is possible to arrange the orientation of the starting attL and attR sites such that, after joining, the two starting nucleic acid segments are separated by a nucleic acid sequence that comprises either an attB site or an attP site.

In some preferred embodiments, the sites may be arranged such that the two starting nucleic acid segments are separated by an attB site after the recombination reaction. In other preferred embodiments, recombination sites from other recombination systems may be used. For example, in some embodiments one or more of the recombination sites may be a lox site or derivative. In some preferred embodiments, recombination sites from more than one recombination system may be used in the same construct. For example, one or more of the recombination sites may be an att site while others may be lox sites. Various combinations of sites from different recombination systems may occur to those skilled in the art and such combinations are deemed to be within the scope of the present invention.

As shown in FIG. 2, nucleic acid segment A (DNA-A) may be flanked by recombination sites having unique specificity, for example attL1 and attL3 sites and nucleic acid segment B (DNA-B) may be flanked by recombination sites attR3 and attL2. For illustrative purposes, the segments are indicated as DNA. This should not be construed as limiting the nucleic acids used in the practice of the present invention to DNA to the exclusion of other nucleic acids. In addition, in this and the subsequent examples, the designation of the recombination sites (i.e., L1, L3, R1, R3, etc.) is merely intend to convey that the recombination sites used have different specificities and should not be construed as limiting the invention to the use of the specifically recited sites. One skilled in the art could readily substitute other pairs of sites for those specifically exemplified.

The attR3 and attL3 sites comprise a reactive pair of sites. Other pairs of unique recombination sites may be used to flank the nucleic acid segments. For example, lox sites could be used as one reactive pair while another reactive pair may be att sites and suitable recombination proteins included in the reaction. Likewise, the recombination sites discussed above can be used in various combinations. In this embodiment, one member of a reactive pair of sites, in this example an LR pair L3 and R3, is present on one nucleic acid segment and the other member of the reactive pair is present on the other nucleic acid segment.

The two segments may be contacted with the appropriate enzymes and a Destination Vector.

The Destination Vector comprises a suitable selectable marker flanked by two recombination sites. In some embodiments, the selectable marker may be a negative selectable marker (such as a toxic gene, e.g., ccdb). One site in the Destination Vector will be compatible with one site present on one of the nucleic acid segments while the other compatible site present in the Destination Vector will be present on the other nucleic acid segment.

Absent a recombination between the two starting nucleic acid segments, neither starting nucleic acid segment has recombination sites compatible with both the sites in the Destination Vector. Thus, neither starting nucleic acid segment can replace the selectable marker present in the Destination Vector.

The reaction mixture may be incubated at about 25° C. for from about 60 minutes to about 16 hours. All or a portion of the reaction mixture will be used to transform competent microorganisms and the microorganisms screened for the presence of the desired construct.

In some embodiments, the Destination Vector comprises a negative selectable marker and the microorganisms transformed are susceptible to the negative selectable marker present on the Destination Vector. The transformed microorganisms will be grown under conditions permitting the negative selection against microorganisms not containing the desired recombination product.

In FIG. 2, the resulting desired product consists of DNA-A and DNA-B separated by an attB3 site and cloned into the Destination Vector backbone. In this embodiment, the same type of reaction (i.e., an LR reaction) may be used to combine the two fragments and insert the combined fragments into a Destination Vector.

In some embodiments, it may not be necessary to control the orientation of one or more of the nucleic acid segments and recombination sites of the same specificity can be used on both ends of the segment.

With reference to FIG. 2, if the orientation of segment A with respect to segment B were not critical, segment A could be flanked by L1 sites on both ends oriented as inverted repeats and the end of segment B to be joined to segment A could be equipped with an R1 site. This might be useful in generating additional complexity in the formation of combinatorial libraries between segments A and B. That is, the joining of the segments can occur in various orientations and given that one or both segments joined may be derived from one or more libraries, a new population or library comprising hybrid molecules in random orientations may be constructed according to the invention.

Although, in the present examples, the recombination between the two starting nucleic acid segments is shown as occurring before the recombination reactions with the Destination Vector, the order of the recombination reactions is not important. Thus, in some embodiments, it may be desirable to conduct the recombination reaction between the segments and isolate the combined segments. The combined segments can be used directly, for example, may be amplified, sequenced or used as linear expression elements as taught by Sykes, et al. (Nature Biotechnology 17:355-359, 1999). In some embodiments, the joined segments may be encapsulated as taught by Tawfik, et al. (Nature Biotechnology 16:652-656, 1998) and subsequently assayed for one or more desirable properties. In some embodiments, the combined segments may be used for in vitro expression of RNA by, for example, including a promoter such as the T7 promoter or SP6 promoter on one of the segments. Such in vitro expressed RNA may optionally be translated in an in vitro translation system such as rabbit reticulocyte lysate.

Optionally, the joined segments may be further reacted with a Destination Vector resulting in the insertion of the combined segments into the vector. In some instances, it may be desirable to isolate an intermediate comprising one of the segments and the vector. For insertion of the segments into a vector, it is not necessary to the practice of the present invention whether the recombination reaction joining the two segments occurs before or after the recombination reaction between the segments and the Destination Vector.

According to the invention, all three recombination reactions preferably occur (i.e., the reaction between segment A and the Destination Vector, the reaction between segment B and the Destination Vector, and the reaction between segment A and segment B) in order to produce a nucleic acid molecule in which both of the two starting nucleic acid segments are now joined in a single molecule. In some embodiments, recombination sites may be selected such that, after insertion into the vector, the recombination sites flanking the joined segments form a reactive pair of sites and the joined segments may be excised from the vector by reaction of the flanking sites with suitable recombination proteins.

With reference to FIG. 2, if the L2 site on segment B is replaced by an L1 site in the opposite orientation with respect to segment B (i.e., the long portion of the box indicating the recombination site was not adjacent to the segment) and the R2 site in the vector were replaced by an R1 site in opposite orientation, the recombination reaction would produce an attP1 site in the vector. The attP1 site would then be capable of reaction with the attB1 site on the other end of the joined segments. Thus, the joined segments could be excised using the recombination proteins appropriate for a BP reaction.

### Example 2

### Simultaneous Cloning of Two Nucleic Acid Fragments Using an LR Reaction to Join the Segments and a BP Reaction to Insert the Segments into a Vector

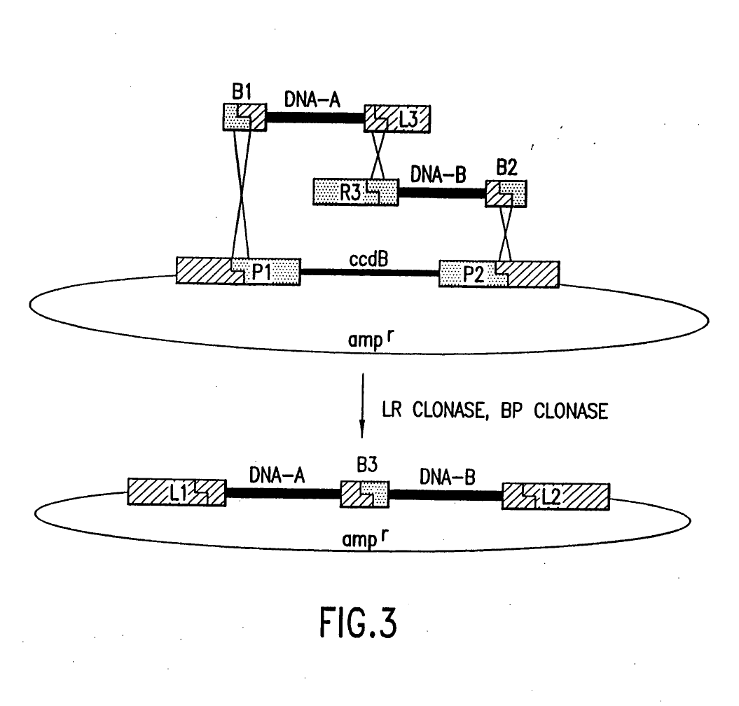

As shown in FIG. 3, a first nucleic acid segment flanked by an attB recombination site and an attL recombination site may be joined to a second nucleic acid segment flanked by an attR recombination site that is compatible with the attL site present on the first nucleic acid segment and flanked by an attB site that may be the same or different as the attB site present on the first segment. FIG. 3 shows an embodiment wherein the two attB sites are different. The two segments may be contacted with a vector containing attP sites in a BP reaction.

A subsequent LR reaction would generate a product consisting of DNA-A and DNA-B separated by either an attP site or an attB site (the product of the LR reaction) and cloned into the vector backbone. In the embodiment shown in FIG. 3, the attL and attR sites are arranged so as to generate an attB site between the segments upon recombination. In other embodiments, the attL and the attR may be oriented differently so as to produce an attP site between the segments upon recombination. In preferred embodiments, after recombination, the two segments may be separated by an attB site.

Those skilled in the art can readily optimize the conditions for conducting the reactions described above without the use of undue experimentation. In a typical reaction from about 50 ng to about 1000 ng of vector may be contacted with the fragments to be cloned under suitable reaction conditions. Each fragment may be present in a molar ratio of from about 25:1 to about 1:25 vector:fragment. In some embodiments, one or more of the fragments may be present at a molar ratio of from about 10:1 to 1:10 vector:fragment. In a preferred embodiment, each fragment may be present at a molar ratio of about 1:1 vector: fragment.

Typically, the nucleic acid may be dissolved in an aqueous buffer and added to the reaction mixture. One suitable set of conditions is 4 μl CLONASE™ enzyme mixture (e.g., Invitrogen Corporation, Cat. Nos. 11791-019 and 11789-013), 4 μl 5× reaction buffer and nucleic acid and water to a final volume of 20 μl. This will typically result in the inclusion of about 200 ng of Int and about 80 ng of IHF in a 20 μl BP reaction and about 150 ng Int, about 25 ng IHF and about 30 ng Xis in a 20 μl LR reaction. Proteins for conducting an LR reaction may be stored in a suitable buffer, for example, LR Storage Buffer, which may comprise 50 mM Tris pH 7.5, 50 mM NaCl, 0.25 mM EDTA, 2.5 mM Spermidine, and 0.2 mg/ml BSA. When stored, proteins for an LR reaction may be stored at a concentration of about 37.5 ng/μl INT, 10 ng/μl IHF and 15 ng/μl XIS. Proteins for conducting a BP reaction may be stored in a suitable buffer, for example, BP Storage Buffer, which may comprise 25 mM Tris pH 7.5, 22 mM NaCl, 5 mM EDTA, 5 mM Spermidine, 1 mg/ml BSA, and 0.0025% Triton X-100. When stored, proteins for an BP reaction may be stored at a concentration of about 37.5 ng/μl INT and 20 ng/μl IHF. One skilled in the art will recognize that enzymatic activity may vary in different preparations of enzymes. The amounts suggested above may be modified to adjust for the amount of activity in any specific preparation of enzymes.

A suitable 5× reaction buffer may comprise 100 mM Tris pH 7.5, 88 mM NaCl, 20 mM EDTA, 20 mM Spermidine, and 4 mg/ml BSA. Thus, in a recombination reaction, the final buffer concentrations may be 20 mM Tris pH 7.5, 17.6 mM NaCl, 4 mM EDTA, 4 mM Spermidine, and 0.8 mg/ml BSA. Those skilled in the art will appreciate that the final reaction mixture may incorporate additional components added with the reagents used to prepare the mixture, for example, a BP reaction may include 0.005% Triton X-100 incorporated from the BP Clonase™.

In some preferred embodiments, particularly those in which attL sites are to be recombined with attR sites, the final reaction mixture may include about 50 mM Tris HCl, pH 7.5, about 1 mM EDTA, about 1 mg/ml BSA, about 75 mM NaCl and about 7.5 mM spermidine in addition to recombination enzymes and the nucleic acids to be combined. In other preferred embodiments, particularly those in which an attB site is to be recombined with an attP site, the final reaction mixture may include about 25 mM Tris HCl, pH 7.5, about 5 mM EDTA, about 1 mg/ml bovine serum albumin (BSA), about 22 mM NaCl, and about 5 mM spermidine.

When it is desired to conduct both a BP and an LR reaction without purifying the nucleic acids in between, the BP reaction can be conducted first and then the reaction conditions adjusted to about 50 mM NaCl, about 3.8 mM spermidine, about 3.4 mM EDTA and about 0.7 mg/ml by the addition of the LR CLONASE™ enzymes and concentrated NaCl. The reaction solution may be incubated at suitable temperature such as, for example, 25° C. for from about 60 minutes to 16 hours. After the recombination reaction, the solution may be used to transform competent host cells and the host cells screened as described above.

One example of a “one-tube” reaction protocol, which facilitates the transfer of PCR products directly to Expression Clones in a two-step reaction performed in a single tube follows. This process can also be used to transfer a gene from one Expression Clone plasmid backbone to another. The Expression Clone is first be linearized within the plasmid backbone to achieve the optimal topology for the BP reaction and to eliminate false-positive colonies due to co-transformation.

Twenty-five μl BP reaction mixture is prepared in a 1.5 ml tube with the following components:

| | | |
|---|---|---|
| attB DNA (100-200 ng) | 1-12.5 | μl |
| attP DNA (pDONR201) 150 ng/μl | 2.5 | μl |
| BP Reaction Buffer | 5.0 | μl |
| TE | to 20 | μl |
| BP Clonase | 5.0 | μl |
| Total vol. | 25 | μl |

The contents of the tube is mixed and incubated for 4 hours, or longer, at 25° C. If the PCR product is amplified from a plasmid template containing selectable markers present on the GATEWAY™ pDONR or pDEST vectors (i.e., kan^{r} or amp^{r}), the PCR product may be treated with the restriction endonuclease DpnI to degrade the plasmid. Such plasmids are a potential source of false-positive colonies in the transformation of GATEWAY™ reactions. Further, when the template for PCR or starting Expression Clone has the same selectable marker as the final Destination Vector (e.g., amp^{r}), plating on LB plates containing 100 μg/ml ampicillin can be used to determine the amount of false positive colonies carried over to the LR reaction step.

Five μl of the reaction mixture is transferred to a separate tube to which is added 0.5 μl Proteinase K Solution. This tube is then incubate for 10 minutes at 37° C. One hundred μl of competent cells are then transformed with 1-2 μl of the mixture and plated on LB plates containing 50 μg/ml kanamycin. This yields colonies for isolation of individual Entry Clones and for assessment of the BP Reaction efficiency.

The following components are added to the remaining 20 μl BP reaction described above:

| | | |
|---|---|---|
| NaCl | 0.75 M | 1 μl |
| Destination Vector | 150 ng/μl | 3 μl |
| LR Clonase | | 6 μl |
| Total vol. | | 30 μl |

The mixture is then incubate at 25° C. for 2 hours, after which 3 μl of proteinase K solution, followed by a further incubation of 10 minutes at 37° C. 1-2 μl of this mixtures is then used to transform 100 μl competent cells, which are then plated on LB plates containing 100 μg/ml ampicillin.

### Example 3

### Cloning of PCR Products Using Fragments by Converting attB Sites into a Reactive Pair of attL and attR Sites in a BP Reaction and Subsequent LR Reaction

A similar strategy to that described in Example 2 can be used to recombine two PCR products and clone them simultaneously into a vector backbone. Since attL and attR sites are 100 and 125 base pairs long, respectively, it may be desirable to incorporate attB sites into the PCR primers since an attB site is 25 base pairs in length. Depending on the orientation of the attB site with respect to the nucleic acid segment being transferred, attB sites can be converted to either an attL or attR site by the BP reaction. Thus, the orientation of the attB site in the attB PCR primer determines whether the attB site is converted to attL or attR. This affords the GATEWAYJ system and methods of the invention great flexibility in the utilization of multiple att sites with unique specificity.

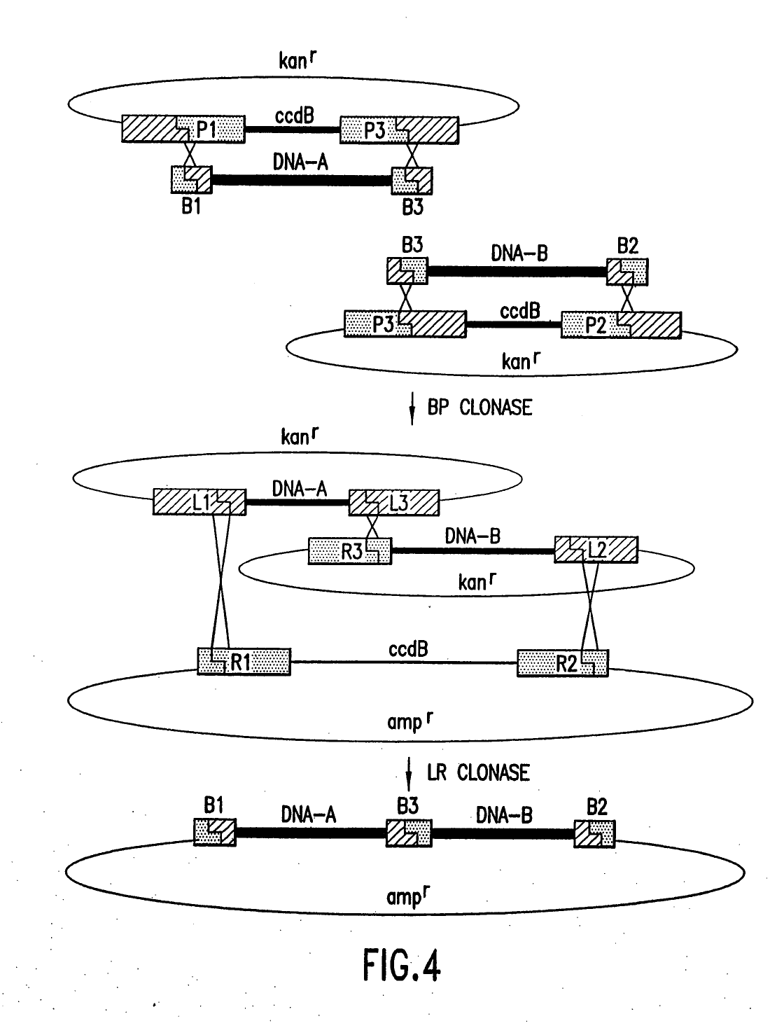

As shown in FIG. 4, two segments (e.g., PCR products) consisting of segment A flanked by mutated attB sites each having a different specificity (e.g., by attB1 and attB3) and segment B flanked by mutated attB sites of different specificity, wherein one of the attB sites present on segment A is the same as one of the attB sites present on segment B (e.g., segment B may contain attB3 and attB2 sites) may be joined and inserted into a vector. The segments may be reacted either individually or together with two attP site containing vectors in a BP reaction. Alternatively, the attP sites might be present on linear segments. One vector contains attP sites compatible with the attB sites present on segment A (e.g., attP1 and attP3 sites). The other vector contains attP sites compatible with the attB sites present on segment B (e.g., attP3 and attP2 sites). When linear segments are used to provide the attP sites, each attP site may be provided on a segment. The orientations of the attB3 and attP3 sites are such that an attR3 site would be generated at the 5=-end of the DNA-B segment and an attL3 site generated at the 3′-end of segment A. The resulting entry clones are mixed with a Destination Vector in a subsequent LR reaction to generate a product consisting of DNA-A and DNA-B separated by an attB3 site and cloned into the Destination Vector backbone.

This basic scheme has been used to link two segments, an attL1-fragment A-attL3 entry clone that is reacted with an attR3-fragment B-attL2 entry clone, and to insert the linked fragments into the destination vector. To generate the appropriate entry clones, two attP Donor vectors were constructed consisting of attP1-ccdB-attP3 and attP3R-ccdB-attP2 such that they could be reacted with appropriate attB PCR products in order to convert the attB sites to attL and attR sites. The designation attP3R is used to indicated that the orientation of the attP3 site is such that reaction with a DNA segment having a cognate attB site will result in the production of an attR site on the segment. This is represented schematically in FIG. 4 by the reversed orientation of the stippled and lined sections of the attB3 on segment B as compared to segment A. On segment B the stippled portion is adjacent to the segment while on segment A the lined portion is adjacent to the segment.

This methodology was exemplified by constructing a DNA segment in which the tetracycline resistance gene (tet) was recombined with the β-galactosidase gene such that the two genes were separated by an attB site in the product. The tet gene was PCR amplified with 5=-attB1 and 3=-attB3 ends. The lacZ gene was PCR amplified with 5=-attB3R and 3=-attB2 ends. The two PCR products were precipitated with polyethylene glycol (PEG). The B1-tet-B3 PCR product was mixed with an attP 1-ccdB-attP3 donor vector and reacted with BP CLONASE™ using a standard protocol to generate an attL1-tet-attL3 entry clone. A correct tet entry clone was isolated and plasmid DNA prepared using standard techniques. In a similar fashion, the attB3R-lacZ-attB2 PCR product was mixed with an attP3R-ccdB-attP2 donor vector and reacted with BP CLONASE™ to generate an attR3-lacZ-attL2 entry clone.

In order to join the two segments in a single vector, an LR CLONASE™ reaction was prepared in a reaction volume of 20 μl containing the following components: 60 ng (25 fmoles) of the supercoiled tet entry clone; 75 ng (20 fmoles) of the supercoiled lacZ entry clone; 150 ng (35 fmoles) of pDEST6 (described in PCT Publication WO 00/52027, the entire disclosure of which is incorporated herein by reference) linearized with NcoI; 4 μl reaction buffer and 4 pI of LR CLONASE™. The final reaction mixture contained 51 mM TrisAHCl, 1 mM EDTA, 1 mg/ml BSA, 76 mM NaCl, 7.5 mM spermidine, 160 ng of Int, 35 ng of IHF and 35 ng of Xis. The reaction was incubated at 25EC overnight and stopped with 2 μl of proteinase K solution (2 mg/ml). A 2 μl aliquot was used to transform 100 μl of E. coli DH5α LE cells and plated on LB plates containing ampicillin and XGal. Approximately 35,000 colonies were generated in the transformation mixture with cells at an efficiency of 1.6×10⁸ cfu/μg of pUC DNA. All the colonies appeared blue indicating the presence of the lacZ gene. 24 colonies were streaked onto plates containing tetracycline and XGal. All of the colonies tested, 24/24, were resistant to tetracycline. 12 colonies were used to inoculate 2 ml of LB broth containing ampicillin for mini preps. 12/12 minipreps contained a supercoiled plasmid of the correct size (7 kb).

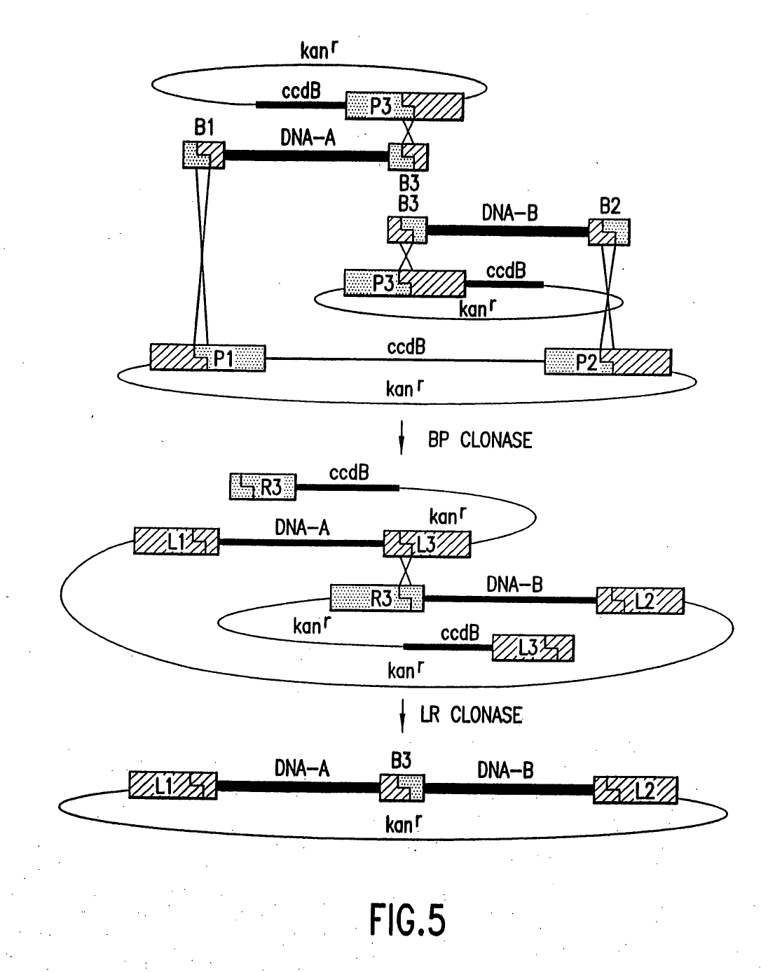

In some embodiments, such as that shown in FIG. 5, two segments can be reacted with a vector containing a single recombination site in order to convert one of the recombination sites on the segments into a different recombination site. In some embodiments, segments containing attB sites may be reacted with a target vector having attP sites. For example, segments A and B are reacted either together or separately with a vector having an attP3 site in order to convert the attB3 sites on the segments into an attL3 and an attR3, respectively. This is done so that the subsequent LR reaction between the two segments results in their being joined by an attB site. The segments may be joined with the attP site containing vector before, simultaneously with or after the recombination reaction to convert the sites to generate a co-integrate molecule consisting of DNA-A flanked by attL1 and attL3 and DNA-B flanked by attR3 and attL2. A subsequent LR reaction will generate a product clone consisting of DNA-A and DNA-B separated by attB3 cloned into a vector backbone.

In some embodiments, an attP site designed to convert the attB used to link the segments to a reactive pair of attL and attR sites may be provided as shorter segments such as restriction fragments, duplexes of synthetic oligonucleotides or PCR fragments. Reactions involving a linear fragment in a BP reaction may require longer incubation times, such a overnight incubation.

The conversion of attB sites to attL or attR sites can also be accomplished solely by PCR. PCR primers containing attL or attR sites can be used to amplify a segment having an attB site on the end. Since the sequence of attL and attR sites contains a portion of the sequence of an attB site, the attB site in this case serves as an overlap region to which the attL or attR PCR primer can anneal. Extension of the annealed attL or attR primer through to the end of the PCR product will generate a fusion template for PCR amplification of the full length PCR product using flanking primers that anneal to the ends of the attL or attR sites. The primers for the PCR reaction may be provided as single stranded oligonucleotides. In some preferred embodiments, the primers may be provided as a duplex, for example, as the product of a PCR reaction to amplify either an attL or attR site.

### Example 4

### Cloning of Two or More Nucleic Acid Fragments into Different Places in the Same Vector

Two or more nucleic acid fragments can be cloned simultaneously into different regions of a vector having multiple sets of recombination sites each flanking a selectable marker. In some embodiments, one or more of the selectable markers may be a negative selectable marker.

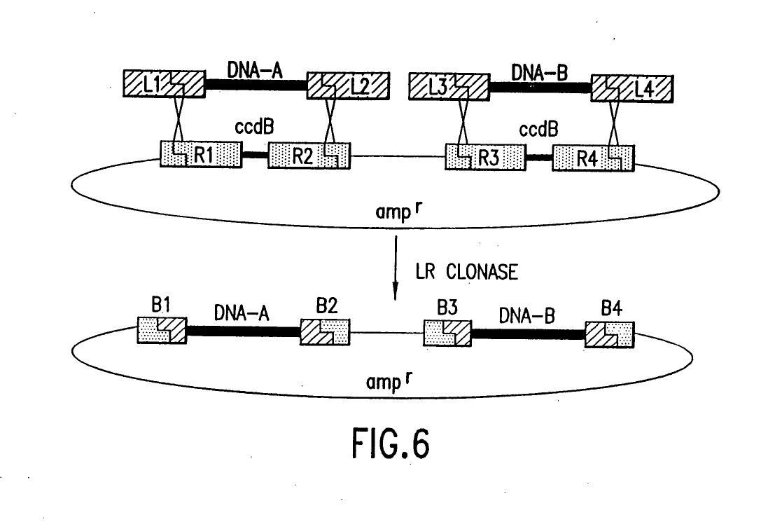

As shown in FIG. 6, two nucleic acid segments A and B which may be present as discrete fragments or as part of a larger nucleic acid molecule such as a plasmid, can be simultaneously cloned into the same destination vector. Nucleic acid segment A (DNA-A) flanked by recombination sites that do not recombine with each other (e.g., attL1 and attL2) and nucleic acid segment B (DNA-B) flanked by recombination sites that do not recombine with each other and do not recombine with the sites flanking segment A (e.g., attL3 and attL4) may be combined with a Destination Vector in an LR reaction. The Destination Vector will contain two pairs of recombination sites, each pair selected to recombine with the sites flanking one of the segments. As an example, FIG. 6 shows two pairs of attR sites (attR1/attR2 and attR3/attR4) each flanking a ccdB negative selectable marker. The three nucleic acids can be combined in a single LR reaction. The resulting product will consist of DNA-A and DNA-B flanked by pairs of attB sites and cloned into distinct regions of the Destination Vector.

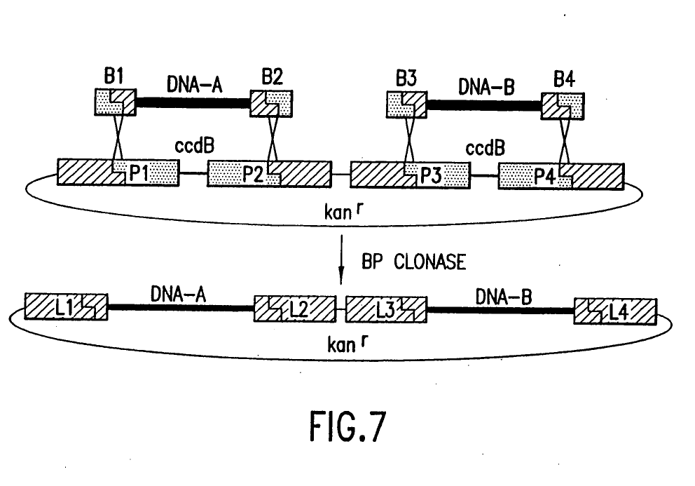

As shown in FIG. 7, an analogous method for inserting nucleic acid segments into a vector can be accomplished using a BP reaction. For example, DNA-A flanked by recombination sites attB1 and attB2 can be combined with DNA-B flanked by recombination sites attB3 and attB4 and a vector containing attP sites in a BP reaction. The resulting product would consist of DNA-A and DNA-B cloned between pairs of attL sites into distinct regions of the vector. In some embodiments, it may be desirable to insert the segments into the target vector sequentially and isolate an intermediate molecule comprising only one of the segments.

It is not necessary that all of the sites be derived form the same recombination system. For example, one segment may be flanked by lox sites while the other segment is flanked by att sites. A segment may have a lox site on one end and an att site on the other end or an frt site on one end. Various combinations of sites may be envisioned by those skilled in the art and such combinations are within the scope of the present invention.

In some embodiments, it may be desirable to isolate intermediates in the reaction shown in FIGS. 6 and 7. For example, it may be desirable to isolate a vector having only one of the segments inserted. The intermediate might be used as is or might serve as the substrate in a subsequent recombination reaction to insert the second segment.

In some embodiments, the present invention is a method of cloning n nucleic acid segments, wherein n is an integer greater than 1, comprising providing n nucleic acid segments, each segment flanked by two unique recombination sites, providing a vector comprising 2n recombination sites wherein each of the 2n recombination sites is capable of recombining with one of the recombination sites flanking one of the nucleic acid segments and conducting a recombination reaction such that the n nucleic acid segments are recombined into the vector thereby cloning the n nucleic acid segments. In further embodiments, the vector comprises n copies of a selectable marker each copy flanked by two recombination sites. In other embodiments, the vector comprises two or more different selectable markers each flanked by two recombination sites. In some embodiments, one or more of the selectable markers may be a negative selectable marker.

In some embodiments, the present invention provides a method of cloning, comprising providing a first, a second and a third nucleic acid segment, wherein the first nucleic acid segment is flanked by a first and a second recombination site, the second nucleic acid segment is flanked by a third and a fourth recombination site and the third nucleic acid segment is flanked by a fifth and a sixth recombination site, wherein the second recombination site is capable of recombining with the third recombination site and none of the first, fourth, fifth or sixth recombination sites is capable of recombining with any of the first through sixth recombination sites, providing a vector comprising a seventh and an eighth recombination site flanking a first selectable marker and comprising a ninth and a tenth recombination site flanking a second selectable marker wherein none of the seventh through tenth recombination sites can recombine with any of the seventh through tenth recombination sites, conducting a first recombination reaction such that the second and the third recombination sites recombine and conducting a second recombination reaction such that the first and the fourth recombination sites recombine with the seventh and the eighth recombination sites respectively and the fifth and the sixth recombination sites recombine with the ninth and the tenth recombination sites thereby cloning the first, second and third nucleic acid segments.

In some embodiments, a nucleic acid segment may comprise a sequence that functions as a promoter. In some embodiments, the first and the second nucleic acid segments may comprise a sequence encoding a polypeptide and the recombination places both polypeptides in the same reading frame. In some embodiments, a nucleic acid segment may comprise a sequence that functions as a transcription termination sequence.

The present invention provides an extremely versatile method for the modular construction of nucleic acids and proteins. Both the inserted nucleic acid segments and the vector can contain sequences selected so as to confer desired characteristics on the product molecules. In those embodiments exemplified in FIGS. 6 and 7, in addition to the inserted segments, one or more of the portions of the vector adjacent to the inserted segments as well as the portion of the vector separating the inserted segments can contain one or more selected sequences.

In some embodiments, the selected sequences might encode ribozymes, epitope tags, structural domains, selectable markers, internal ribosome entry sequences, promoters, enhancers, recombination sites and the like. In some preferred embodiments, the portion of the vector separating the inserted segments may comprise one or more selectable markers flanked by a reactive pair of recombination sites in addition to the recombination sites used to insert the nucleic acid segments.

This methodology will be particularly well suited for the construction of gene targeting vectors. For example, the segment of the vector between the pairs of recombination sites may encode one or more a selectable markers such as the neomycin resistance gene. Segments A and B may contain nucleic acid sequences selected so as to be identical or substantially identical to a portion of a gene target that is to be disrupted. After the recombination reaction, the Destination Vector will contain two portions of a gene of interest flanking a positive selectable marker. The vector can then be inserted into a cell using any conventional technology, such as transfection, whereupon the portions of the gene of interest present on the vector can recombine with the homologous portions of the genomic copy of the gene. Cells containing the inserted vector can be selected based upon one or more characteristics conferred by the selectable marker, for example, in the case when the selectable marker is the neomycin resistance gene, their resistance to G-418.

In some embodiments, one or more a negative selectable markers may be included in the portion of the Destination Vector that does not contain the target gene segments and the positive selectable marker. The presence of one or more negative selectable markers permits the selection against cells in which the entire Destination Vector was inserted into the genome or against cells in which the Destination Vector is maintained extrachromosomally.

In some preferred embodiments, additional recombination sites may be positioned adjacent to the recombination sites used to insert the nucleic acid segments. Molecules of this type will be useful in gene targeting application where it is desirable to remove the selectable marker from the targeted gene after targeting, the so called “hit and run” methodology. Those skilled in the art will appreciate that the segments containing homologous sequence need not necessarily correspond to the sequence of a gene. In some instances, the sequences may be selected to be homologous to a chromosomal location other than a gene.

This methodology is also well suited for the construction of bi-cistronic expression vectors. In some embodiments, expression vectors containing bi-cistronic expression elements where two structural genes are expressed from a single promoter and are separated by an internal ribosome entry sequence (IRES, see Encarnación, Current Opinion in Biotechnology 10:458-464 (1999), specifically incorporated herein by reference). Such vectors can be used to express two proteins from a single construct.

In some embodiments, it may not be necessary to control the orientation of one or more of the nucleic acid segments and recombination sites of the same specificity can be used on both ends of the segment. With reference to FIG. 6, if the orientation of segment A with respect to segment B were not critical, segment A could be flanked by L1 sites on both ends and the vector equipped with two R1 sites. This might be useful in generating additional complexity in the formation of combinatorial libraries between segments A and B.

### Example 5

### Combining Multiple Fragments into a Single Site in a Vector

In some embodiments, the present invention provides a method of cloning n nucleic acid segments, wherein n is an integer greater than 1, comprising providing a 1^{st} through an n^{th} nucleic acid segment, each segment flanked by two unique recombination sites, wherein the recombination sites are selected such that one of the two recombination sites flanking the i^{th} segment, nᵢ, reacts with one of the recombination sites flanking the nᵢ₋₁th segment and the other recombination site flanking the i^{th} segment reacts with one of the recombination sites flanking the nᵢ₊₁th segment, providing a vector comprising at least two recombination sites wherein one of the two recombination sites on the vector reacts with one of the sites on the 1^{st} nucleic acid segment and another site on the vector reacts with a recombination site on the n^{th} nucleic acid segment. It is a further object of the present invention to provide a method of cloning, comprising providing a first, a second and a third nucleic acid segment, wherein the first nucleic acid segment is flanked by a first and a second recombination site, the second nucleic acid segment is flanked by a third and a fourth recombination site and the third nucleic acid segment is flanked by a fifth and a sixth recombination site, wherein the second recombination site is capable of recombining with the third recombination site and the fourth recombination site is capable of recombining with the fifth recombination site, providing a vector having at least a seventh and an eighth recombination site such that the seventh recombination site is capable of reacting with the first recombination site and the eighth recombination site is capable of reacting with the sixth recombination site and conducting at least one recombination reaction such that the second and the third recombination sites recombine, the fourth and the fifth recombination sites recombine, the first and the seventh recombination sites recombine and the sixth and the eighth recombination sites recombine thereby cloning the first, second and third nucleic acid segments. In some embodiments, at least one nucleic acid segment comprises a sequence that functions as a promoter.

In some embodiments, at least two nucleic acid segments comprise sequences encoding a polypeptide and the recombination places both polypeptides in the same reading frame. In some embodiments, at least one nucleic acid segment comprises a sequence that functions as a transcription termination sequence. In some embodiments, at least one fragment comprises an origin of replication. In some embodiments, at least one fragment comprises a sequence coding for a selectable marker.

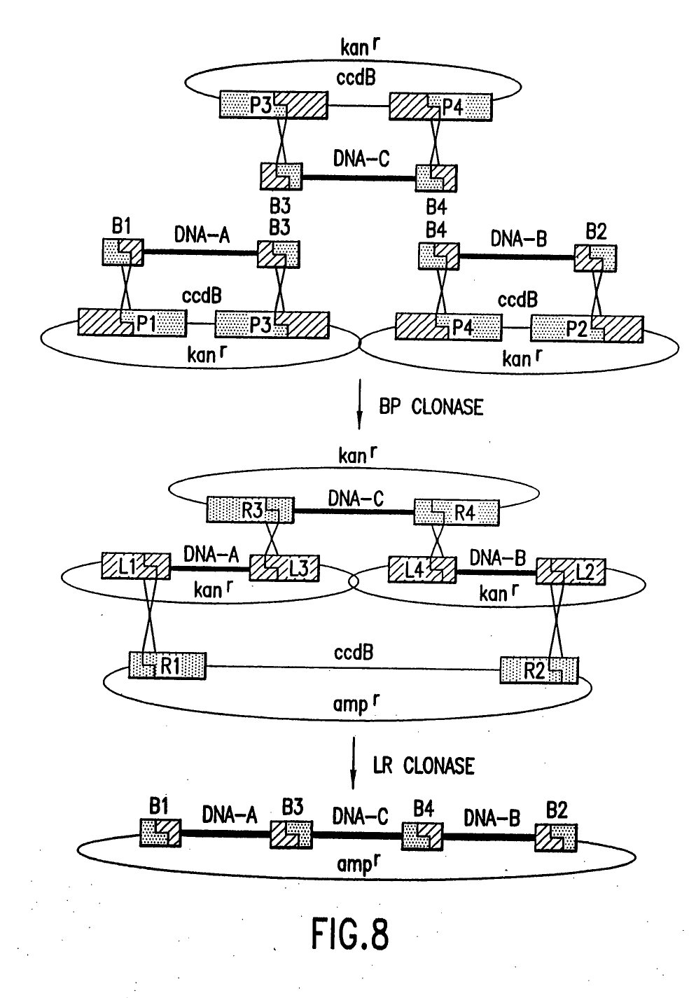

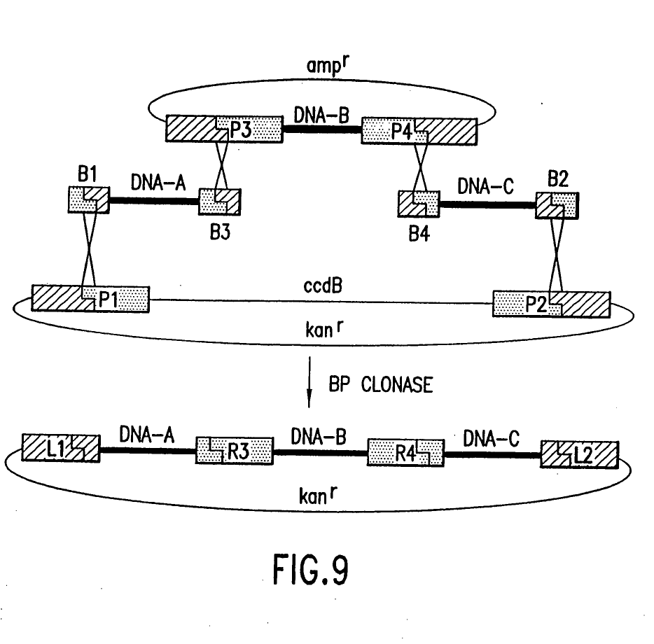

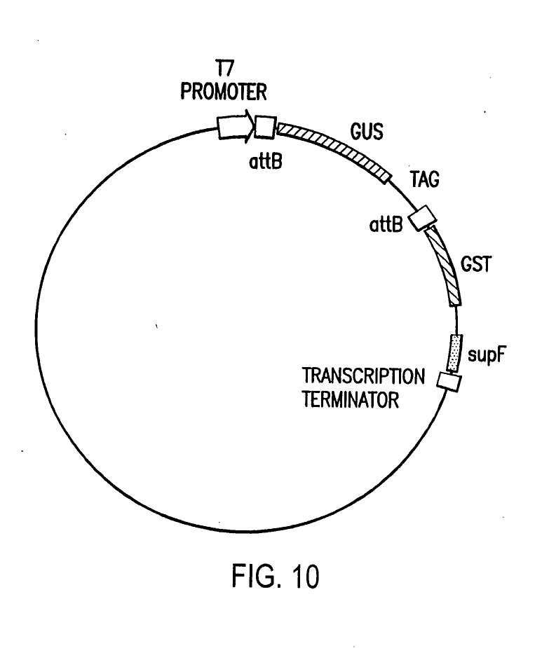

This embodiment is exemplified in FIGS. 8 and 9 for the case when n=3. In this embodiment, the present invention provides a method of cloning, comprising providing a first, a second and a third nucleic acid segment, wherein the first nucleic acid segment is flanked by a first and a second recombination site, the second nucleic acid segment is flanked by a third and a fourth recombination site and the third nucleic acid segment is flanked by a fifth and a sixth recombination site, wherein the second recombination site is capable of recombining with the third recombination site and the fourth recombination site is capable of recombining with the fifth recombination site, providing a vector comprising a seventh and an eighth recombination site and conducting at least one recombination reaction such that the second and the third recombination sites recombine and the fourth and the fifth recombination sites recombine and the first and the sixth recombination sites recombine with the seventh and the eighth recombination sites respectively, thereby cloning the first, second and third nucleic acid segments.

As discussed above, when the orientation of a given segment is not critical, the invention may be modified by placing recombination sites having the same specificity on both ends of the given segment and adjusting the recombination sites of the adjacent segments and/or the recombination sites in the vector accordingly.

In addition to the utilities discussed above for the combination of two fragments in a single vector, embodiments of this type will be useful for the construction of vectors from individual fragments containing various functions. Thus, the invention provides a modular method for the construction of vectors.

In some embodiments, at least one nucleic acid segment comprises a sequence that functions as a promoter. In some embodiments, at least two nucleic acid segments comprise a sequence encoding a polypeptide and the recombination places both polypeptides in the same reading frame. In some embodiments, at least one nucleic acid segment comprises a sequence that functions as a transcription termination sequence. In some embodiments, at least one fragment comprises an origin of replication. In some embodiments, at least one fragment comprises a sequence coding for a selectable marker. In some embodiments, a fragment may comprise sequence coding for more than one function. In some embodiments, a fragment may comprise sequence coding for an origin of replication and sequence encoding a selectable marker.

When multiple nucleic acid segments are inserted into vectors using methods of the invention, expression of these segments may be driven by the same regulatory sequence or different regulatory sequences

### Example 6

### Use of Suppressor tRNAs to Generate Fusion Proteins

The recombinational cloning techniques described above permit the rapid movement of a first nucleic acid segment encoding a protein, e.g., M, from one nucleic acid molecule to one or more second and/or third nucleic acid molecules. Because the recombination event is site specific, the orientation and reading frame of the nucleic acid segment can be controlled with respect to the second nucleic acid molecules, e.g., a vector. This control makes the construction of fusions between sequences present on the nucleic acid segment encoding a protein and sequences present on the second nucleic acid molecule a simple matter.

In general terms, a gene may be expressed in four forms: native at both amino and carboxy termini, modified at either end, or modified at both ends. A construct containing a gene of interest may include the N-terminal methionine ATG codon, and a stop codon at the carboxy end, of the open reading frame, or ORF, thus ATG-ORF-stop. Frequently, the gene construct will include translation initiation sequences, tis, that may be located upstream of the ATG that allow expression of the gene, thus tis-ATG-ORF-stop. Constructs of this sort allow expression of a gene as a protein that contains the same amino and carboxy amino acids as in the native, uncloned, protein. When such a construct is fused in-frame with an amino-terminal protein tag, e.g., GST (or any other protein), the tag will have its own tis, thus tis-ATG-tag-tis-ATG-ORF-stop, and the bases comprising the tis of the ORF will be translated into amino acids between the tag and the ORF. In addition, some level of translation initiation may be expected in the interior of the mRNA (i.e., at the ORF=s ATG and not the tag=s ATG) resulting in a certain amount of native protein expression contaminating the desired protein.

DNA (lower case): tis1-atg-tag-tis2-atg-orf-stop

RNA (lower case, italics): tis1-atg-tag-tis2-atg-orf-stop

Protein (upper case): ATG-TAG-TIS2-ATG-ORF (tis1 and stop are not translated)+contaminating ATG-ORF (translation of ORF beginning at tis2).

Using recombinational cloning, it is a simple matter for those skilled in the art to construct a vector containing a tag adjacent to a recombination site permitting the in frame fusion of a tag to the C- and/or N-terminus of the ORF of interest. Using the methods provided, one of skill in the art may maximize the number of ways a single cloned gene can be expressed without the need to manipulate the gene construct itself. Specifically, the present invention provides materials and methods for the controlled expression of a C- and/or N-terminal fusion to a gene of interest using one or more suppressor tRNAs to suppress the termination of translation at a stop codon. Thus, the present invention provides materials and methods in which a gene construct is prepared flanked with recombination sites.

The construct is prepared with a sequence coding for a stop codon preferably at the C-terminus of the gene encoding the protein of interest, e.g., M. In some embodiments, a stop codon can be located adjacent to the gene, for example, within the recombination site flanking the gene. The target gene construct can be transferred through recombination to various vectors which can provide various C-terminal or N-terminal tags (e.g., GFP, GST, His Tag, GUS, Protein A, Protein G, etc.) to the gene of interest of provide genes encoding other second, third or fourth proteins, e.g., M, E, or M1. When the stop codon is located at the carboxy terminus of the gene, expression of the gene with a “native” carboxy end amino acid sequence occurs under non-suppressing conditions (i.e., when the suppressor tRNA is not expressed) while expression of the gene as a carboxy fusion protein occurs under suppressing conditions. The present invention is exemplified using an amber suppressor supF, which is a particular tyrosine tRNA gene (tyrT) mutated to recognize the UAG stop codon. Those skilled in the art will recognize that other suppressors and other stop codons could be used in the practice of the present invention.

In the example provided, the gene coding for the suppressing tRNA has been incorporated into the vector from which the gene of interest is to be expressed. In other embodiments, the gene for the suppressor tRNA may be in the genome of the host cell. In still other embodiments, the gene for the suppressor may be located on a separate vector and provided in trans. In embodiments of this type, the vector containing the suppressor gene may have an origin of replication selected so as to be compatible with the vector containing the gene construct. The selection and preparation of such compatible vectors is within ordinary skill in the art. Those skilled in the art will appreciate that the selection of an appropriate vector for providing the suppressor tRNA in trans may include the selection of an appropriate antibiotic resistance marker. For example, if the vector expressing the target gene contains an antibiotic resistance marker for one antibiotic, a vector used to provide a suppressor tRNA may encode resistance to a second antibiotic. This permits the selection for host cells containing both vectors.

In some preferred embodiments, more than one copy of a suppressor tRNA may be provided in all of the embodiments described above. For example, a host cell may be provided that contains multiple copies of a gene encoding the suppressor tRNA. Alternatively, multiple gene copies of the suppressor tRNA under the same or different promoters may be provided in the same vector background as the target gene of interest. In some embodiments, multiple copies of a suppressor tRNA may be provided in a different vector than the one use to contain the target gene of interest. In other embodiments, one or more copies of the suppressor tRNA gene may be provided on the vector containing the gene for the protein of interest and/or on another vector and/or in the genome of the host cell or in combinations of the above. When more than one copy of a suppressor tRNA gene is provided, the genes may be expressed from the same or different promoters which may be the same or different as the promoter used to express the gene encoding the protein of interest.

In some embodiments, two or more different suppressor tRNA genes may be provided. In embodiments of this type one or more of the individual suppressors may be provided in multiple copies and the number of copies of a particular suppressor tRNA gene may be the same or different as the number of copies of another suppressor tRNA gene. Each suppressor tRNA gene, independently of any other suppressor tRNA gene, may be provided on the vector used to express the gene of interest and/or on a different vector and/or in the genome of the host cell. A given tRNA gene may be provided in more than one place in some embodiments. For example, a copy of the suppressor tRNA may be provided on the vector containing the gene of interest while one or more additional copies may be provided on an additional vector and/or in the genome of the host cell. When more than one copy of a suppressor tRNA gene is provided, the genes may be expressed from the same or different promoters which may be the same or different as the promoter used to express the gene encoding the protein of interest and may be the same or different as a promoter used to express a different tRNA gene.

In some embodiments of the present invention, the gene of interest and the gene expressing the suppressor tRNA may be controlled by the same promoter. In other embodiments, the gene of interest may be expressed from a different promoter than the suppressor tRNA. Those skilled in the art will appreciate that, under certain circumstances, it may be desirable to control the expression of the suppressor tRNA and/or the target gene of interest using a regulatable promoter. For example, either the gene of interest and/or the gene expressing the suppressor tRNA may be controlled by a promoter such as the lac promoter or derivatives thereof such as the tac promoter. In the embodiment shown, both the gene of interest and the suppressor tRNA gene are expressed from the T7 RNA polymerase promoter. Induction of the T7 RNA polymerase turns on expression of both the gene of interest (GUS in this case) and the supF gene expressing the suppressor tRNA as part of one RNA molecule.

In some preferred embodiments, the expression of the suppressor tRNA gene may be under the control of a different promoter from that of the gene of interest. In some embodiments, it may be possible to express the suppressor gene before the expression of the gene of interest. This would allow levels of suppressor to build up to a high level, before they are needed to allow expression of a fusion protein by suppression of a the stop codon. For example, in embodiments of the invention where the suppressor gene is controlled by a promoter inducible with IPTG, the gene of interest is controlled by the T7 RNA polymerase promoter and the expression of the T7 RNA polymerase is controlled by a promoter inducible with an inducing signal other than IPTG, e.g., NaCl, one could turn on expression of the suppressor tRNA gene with IPTG prior to the induction of the T7 RNA polymerase gene and subsequent expression of the gene of interest. In some preferred embodiments, the expression of the suppressor tRNA might be induced about 15 minutes to about one hour before the induction of the T7 RNA polymerase gene. In a preferred embodiment, the expression of the suppressor tRNA may be induced from about 15 minutes to about 30 minutes before induction of the T7 RNA polymerase gene. In the specific example shown, the expression of the T7 RNA polymerase gene is under the control of a salt inducible promoter. A cell line having an inducible copy of the T7 RNA polymerase gene under the control of a salt inducible promoter is commercially available from Invitrogen Corporation, Carlsbad, Calif., under the designation of the BL21 SI strain.

In some preferred embodiments, the expression of the target gene of interest and the suppressor tRNA can be arranged in the form of a feedback loop. For example, the gene of interest may be placed under the control of the T7 RNA polymerase promoter while the suppressor gene is under the control of both the T7 promoter and the lac promoter, and the T7 RNA polymerase gene itself is transcribed by both the T7 promoter and the lac promoter, and the T7 RNA polymerase gene has an amber stop mutation replacing a normal tyrosine stop codon, e.g., the 28^{th} codon (out of 883). No active T7 RNA polymerase can be made before levels of suppressor are high enough to give significant suppression. Then expression of the polymerase rapidly rises, because the T7 polymerase expresses the suppressor gene as well as itself. In other preferred embodiments, only the suppressor gene is expressed from the T7 RNA polymerase promoter. Embodiments of this type would give a high level of suppressor without producing an excess amount of T7 RNA polymerase. In other preferred embodiments, the T7 RNA polymerase gene has more than one amber stop mutation. This will require higher levels of suppressor before active T7 RNA polymerase is produced.

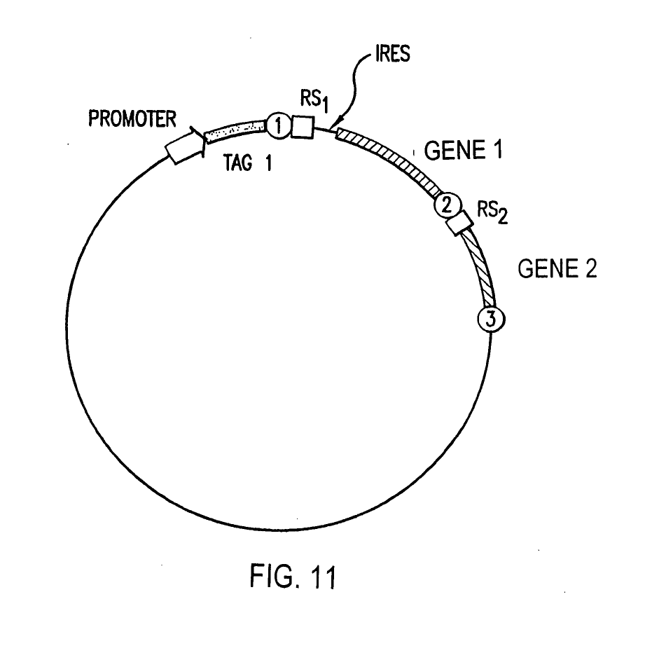

In some embodiments of the present invention it may be desirable to have more than one stop codon suppressible by more than one suppressor tRNA. With reference to FIG. 11, a vector may be constructed so as to permit the regulatable expression of N- and/or C-terminal fusions of a protein of interest from the same construct. A first tag sequence, TAG 1 in FIG. 11, is expressed from a promoter represented by an arrow in the figure. The tag sequence includes a stop codon in the same reading frame as the tag. The stop codon 1, may be located anywhere in the tag sequence and is preferably located at or near the C-terminal of the tag sequence. The stop codon may also be located in the recombination site RSI or in the internal ribosome entry sequence (IRES). The construct also includes a gene of interest (GENE1) which includes a stop codon 2. The first tag and the gene of interest are preferably in the same reading frame although inclusion of a sequence that causes frame shifting to bring the first tag into the same reading frame as the gene of interest is within the scope of the present invention. Stop codon 2 is in the same reading frame as the gene of interest and is preferably located at or near the end of the coding sequence for the gene. Stop codon 2 may optionally be located within the recombination site RS₂. The construct also includes a second sequence of a gene of interest, GENE2, in the same reading frame as the gene of interest indicated by TAG2 in FIG. 11 and the second tag sequence may optionally include a stop codon 3 in the same reading frame as the second tag. A transcription terminator may be included in the construct after the coding sequence of the second tag (not shown in FIG. 11). Stop codons 1, 2 and 3 may be the same or different. In some embodiments, stop codons 1, 2 and 3 are different. In embodiments where 1 and 2 are different, the same construct may be used to express an N-terminal fusion, a C-terminal fusion and the native protein by varying the expression of the appropriate suppressor tRNA. For example, to express the native protein, no suppressor tRNAs are expressed and protein translation is controlled by the IRES. When an N-terminal fusion is desired, a suppressor tRNA that suppresses stop codon 1 is expressed while a suppressor tRNA that suppresses stop codon 2 is expressed in order to produce a C-terminal fusion. In some instances it may be desirable to express a doubly tagged protein of interest in which case suppressor tRNAs that suppress both stop codon 1 and stop codon 2 may be expressed.

In one specific aspect, the present invention provides product nucleic acid molecules formed by recombinational joining of one or more segments encoding proteins having known or predetermined molecular weights and/or pI. The proteins may have the same or different molecular weight and/or p1. As an example, the methods of the invention may be used to join four segments (n=4) encoding protein M, and a segment encoding protein E, each segment comprising a suppressible stop codon. The product nucleic in such a case may encode a fusion protein E-L-L-L-L, if all the stop codons are suppressed. However, by strategically manipulating the transcriptional control elements such as the promoter and gene encoding suppressor tRNA so that under certain conditions, one or more stop codons are not suppressed, one of skill in the art would be able to a generate fusion proteins comprising E-L, E-L-L, E-L-L-L, or E-L-L-L-L from one product nucleic acid molecule.

### Example 7

### Recombinant Western Standard Proteins

Plasmid clones expressing recombinant molecular weight standard proteins were obtained. The plasmid clones expressing the 40 kDa, 60 kDa and 100 kDa recombinant proteins are p2905, p2885 and p2894, respectively. Suitable nucleotide sequences for use in the present invention are provided in Table 6. Suitable amino acid sequences are provided in Table 7. The nucleotide sequences encoding the 40 kDa, 60 kDa, and 100 kDa recombinant proteins and their amino acid sequences are provided in Tables 6 and 7 respectively.

PCR Amplification of Standard Protein ORFs

Each of the selected ORFs were PCR amplified with specific flanking att B sites. To PCR amplify the 40 kDa ORF with flanking attB1 and attB3 sites the PCR primers L140 (SEQ ID NO: 1) and L340 (SEQ ID NO: 2) were used. To PCR amplify the 60 kDa ORF with flanking attB3 and attB4 sites the PCR primers R360 (SEQ ID NO:3) and R460 (SEQ ID NO: 4) were used. To PCR amplify the 100 kDa ORF with flanking attB4 and attB2 sites the PCR primers L4100 (SEQ ID NO:5) and L2100 (SEQ ID NO:6) were used. All PCR fragments were gel purified prior to their use in the BP Clonase reactions.

| | |
|---|---|
| PCR | |
| Primer | Sequence |

| | | |
|---|---|---|
| L140 | GGGACAACTTTGTACAAAAAAGTTGAT | (SEQ ID NO. 45) |
| | GACCATGATTAGGATTCACTGGCCGT | |
| L340 | GGGGACAACTTTGTATAATAAAGTTCA | (SEQ ID NO. 46) |
| | TCACCCGGGCTACAGTGATGATGGTGG | |
| | TGAT | |
| R360 | GGGGACAACTTTATTATACAAAGTTTG | (SEQ ID NO. 47) |
| | ATGACCATGATTACGGATTCACTGGCC | |
| | GT | |
| R460 | GGGGACAACTTTTCTATACAAAGTTCA | (SEQ ID NO. 48) |
| | TCACCCGGGCTACAGTGATGATGGTGG | |
| | TG | |
| L4100 | GGGGACAACTTTGTATAGAAAAGTTTG | (SEQ ID NO. 49) |
| | ATGACCATGATTACGGATTCACTGGCC | |
| | GT | |
| L2100 | GGGGACAACTTTGTACAAGAAAGTTCT | (SEQ ID NO. 50) |
| | CAATCACCCGGGCTGCAGTGATGATGG | |
| | TG | |

Sequences of the PCR primers used to amplify the ORFs with flanking attB sites. The attB site sequences are shown in bold.

BP Clonase Reactions

The PCR amplified 40 kDa, 60 kDa and 100 kDa ORFs were recombined with the Donor vectors pDONR208A, pDONR213B and pDONR214C, respectively. These reactions generated the Entry clones pENTR208-40, pENTR213B-60 and pENTR214C-100. The BP Clonase reactions contained 150 ng of gel purified PCR product, 300 ng of Donor vector and 4:1 of BP Clonase (Invitrogen Corp., Carlsbad, Calif., Catalog Nos. 11789-013 and 11789-021 (online at www.invitrogen.com)) in a final volume of 20:1. The reaction was performed between 22° C. to 25° C. for one hour and terminated with the addition of Proteinase K. 2:1 of the BP Clonase reaction was transformed into TOP10 cells and plated onto Kanamycin LB-agar plates. Colonies generated were amplified in liquid media for plasmid DNA purification. Restriction enzyme digest analysis was used to select for the correct clones from the BP Clonase reactions.

MultiSite Gateway LR Clonase Reactions

The first reaction was performed in a final volume of 20 ul in IX LR Clonase Plus Buffer with 20 fmoles of pBAD-Dest49, 20 fmoles of each Entry clone, pENTR208-40, pENTR213B-60 and pENTR214C-100, and 4:1 of LR Clonase enzyme mix (Invitrogen Corp., Carlsbad, Calif., Catalog Nos. 11791-019 and 11781-043 (online at www.invitrogen.com)). The 5× concentration LR Clonase buffer comprises 200 mM Tris-HCl, pH 7.5, 5 mM EDTA, 40 mM Spermidine, 320 mM NaCl and 5 mg/ml BSA (Sigma-Aldrich Co., Catalog No. A-3059 (online at www.sigmaaldrich.com)).

The reaction was incubated at room temperature for 18 hours. 2:1 of the MultiSite Gateway LR Clonase reaction was transformed into TOP10 cells and plated onto Ampicillin LB-agar plates. Colonies generated were amplified in liquid media for plasmid DNA purification. Restriction enzyme digest analysis was used to select for the correct clones from the MultiSite Gateway LR Clonase reaction.

Expression of the Expression Clone

Expression clones were grown to saturation in Ampicillin LB media at 37° C. with shaking. A 200:1 aliquot of this culture was used to inoculate 5 ml of Ampicillin LB media and allowed to grow at 37° C. with shaking until an OD₆₀₀ of 0.5 was reached. Protein expression was induced with the addition of arabinose to a final concentration of 0.2% (w/v) and the culture was allowed to grow for a further 4 hours at 37° C. with shaking. 1.5 ml of the culture was harvested by centrifugation and the supernatant discarded. The cell pellet was resuspended in 600:1 of BugBuster (Novagen, Inc., Madison, Wis., Cat. No. 70921-3 (online at www.novagen.com)) and allowed to incubate at room temperature for 30 minutes. 150 ul of the lysate was mixed with 50:1 of 4× Sample buffer (with DTT) and incubated at 70° C. for 10 minutes, 10 ul of this mixture was separated by polyacrylamide gel electrophoresis and proteins visualized by Coomassie Blue staining. NuPAGE gels (Invitrogen, Corp., Carlsbad, Calif., Cat. No. NUO301) were used to resolve the protein lysates according to supplied instructions. Protein staining was achieved with SimplyBlue SafeStain.

Western Transfer and Detection of the Recombinant 200 kDa MagicMark Protein

The Western transfer of proteins onto PVDF membranes from NuPAGE gels was performed according to the NuPAGE Technical Guide. Detection of proteins transferred onto PVDF membranes was performed according to the instructions supplied with the WesternBreeze Chemiluminescent Western Blot Immunodetection Kit (Invitrogen, Corp., Carlsbad, Calif., Cat. No. WB7104).

Results and Discussion

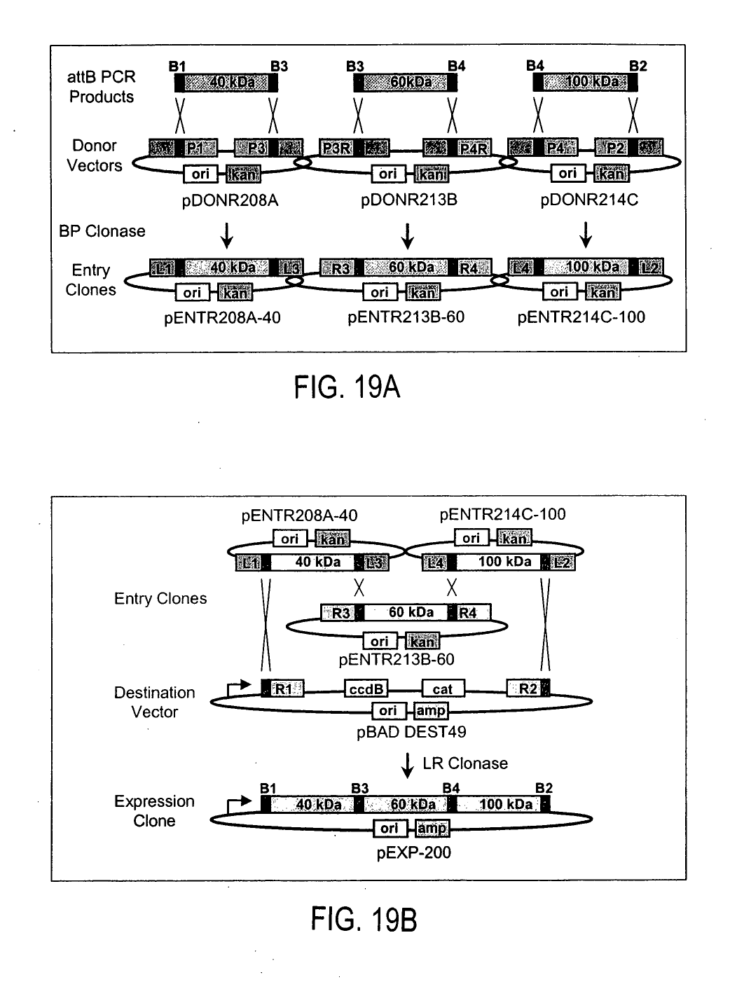

The 200 kDa protein was designed as a fusion protein of the 40 kDa, 60 kDa and 100 kDa proteins. These ORFs were PCR amplified, cloned by BP Clonase reactions (FIG. 19A) and linked genetically in a specific order with a Clonase reaction (FIG. 19B) onto an E. coli expression vector, pBAD Dest49. The expressed recombinant 200 kDa protein had to fulfill three conditions for it to be considered as a molecular marker molecule.

1) The expressed recombinant protein has to migrate in a NuPAGE gel at approximately 200 kDa.

2) The expressed recombinant protein has to be present in significant amounts in the expression induced lysate.

3) The expressed recombinant protein has to be detected by the alkaline phosphatase-conjugated secondary antibody supplied in the WesternBreeze Chemiluminescent Western Blot Immunodetection Kit

Cloning of Entry Clones and the 200 kDa Expression Clone

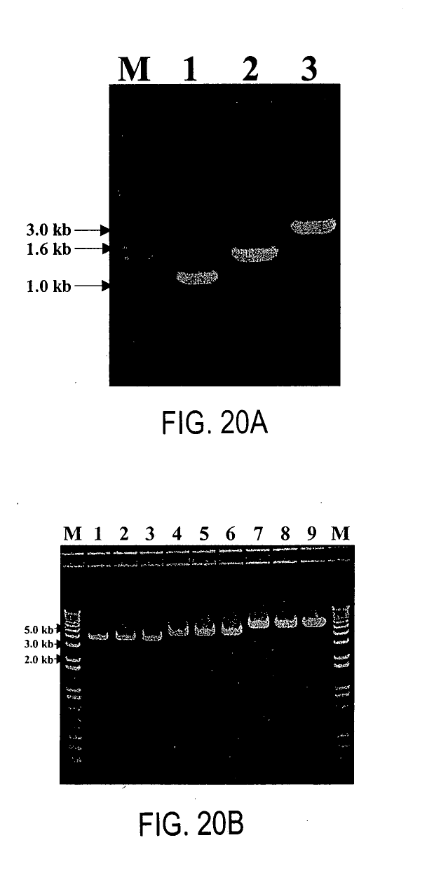

The 40 kDa, 60 kDa and 100 kDa ORFs were PCR amplified with flanking att sites and cloned into their respective Donor vector bearing corresponding attP sites (FIG. 19A). The gel purified PCR amplified ORFs are depicted in FIG. 20A. The efficiency of the BP Clonase reactions with these PCR amplified fragments was 100%. This was determined by assessing the plasmid DNA isolated from colonies generated from the transformation of the BP Clonase reactions which revealed that all nine clones examined were the desired recombinant clones (FIG. 20B).

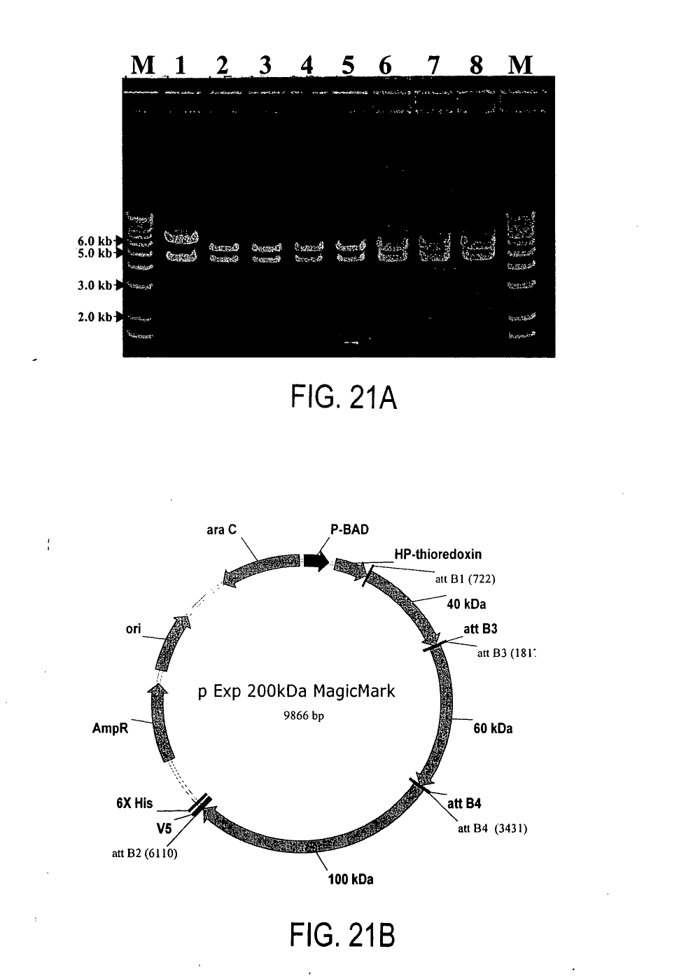

The Entry clones were assembled onto pBAD Dest49 (Invitrogen, Corp., Carlsbad, Calif., Cat. No. 12283016) using a MultiSite Gateway LR Clonase reaction to generate the 200 kDa expression construct (FIG. 19B). Plasmid DNA purified from colonies generated from the transformation of the MultiSite LR Clonase reaction were examined by restriction enzyme digestion and revealed that seven of the eight selected clones were the desired construct (FIG. 21A). The final expression clone is depicted in FIG. 21B.

Expression and Detection of the 200 kDa MagicMark Protein

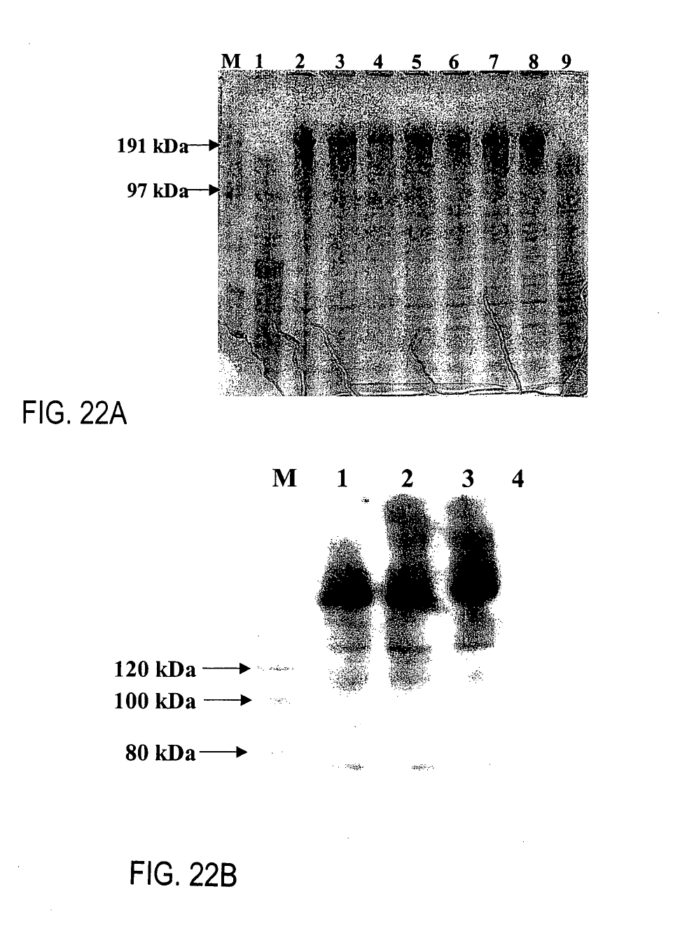

The seven 200 kDa expression clones confirmed by the restriction analysis seen in FIG. 21A were evaluated for their ability to express a 200 kDa recombinant protein. After induction with arabinose, all seven clones expressed a recombinant protein of 200 kDa (FIG. 22A, lanes 2 to 8). The expression lysates of three of these seven expression clones (expression clones chosen are seen in FIG. 21A, lanes 2 to 4 and FIG. 22A, lanes 2 to 4) were subjected to further testing to determine if the expressed 200 kDa protein contained Protein G domains that bound IgG molecules. As seen in FIG. 22B, a protein of molecular weight larger than 120 kDa bound the alkaline phosphatase-conjugated secondary antibody supplied in the WesternBreeze Chemiluminescent western Blot Immunodetection Kit, indicating that the expressed 200 kDa protein possesses at least one IgG binding domain.

Conclusions

The recombinant 200 kDa protein described above fulfills the criteria set out for the recombinant protein to be considered as a molecular marker molecule. The expressed recombinant protein migrates at about 200 kDa in a NuPAGE gel (FIG. 22A, lanes 2 to 8), it is highly expressed in TOP10 cells after induction with arabinose (FIG. 22A, lanes 2 to 8), and the reagents supplied with the WesternBreeze Chemiluminescent Western Blot Immunodetection Kit are able to detect the recombinant 200 kDa protein (FIG. 22B, lanes 1 to 3).

The present invention has been described in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

TABLE 6

Nucleotide Sequences of Open Reading Frames Encoding Various
Sized Protein Fragments

| | |
|---|---|
| | Molecular |
| Nucleotide sequence | Weight |

| | | |
|---|---|---|
| ATGACCATGATTACGGATTCACTGGCCGTCGTTTTACAACGTCGTGACTGGT | (SEQ ID NO: 51) | 20 kDa |
| ACAACCCTGGCGTTACCCAACTTAATCACCCCGTGGACTTCCAGTTCAACAT | | |
| CAGCCGCTACAGTCAACAGCAACTGATGGAAACCAGCCATCGCCATCTGCT | | |
| GCACGCGGAAGAAGGCACATGGCTGAATATCGACGGTTTCCATATGGGGAT | | |
| TGGTGGCGACGACTCCTGGAGCCCGTCAGTATCGGCGGTATTCCAGCTGA | | |
| GCGGCGCAATCGAGGGTAGGGTCGCGACCATGGAATTCGATGCGTCTGAA | | |
| TTAACAGATGCCTCGACAACTTACAAACTTGTTATTAATGGTAAAACATTGAA | | |
| AGGCGGAACAACTACTGAAGCTGTTGATGCTGCTACTGCAGAAAAAGTCTTC | | |
| AAACAATACGCtaacgacaacggtgttgacggtgaatggacttacgacgatgc | | |
| gactaagatctttacagttactgaaaaactgatcgaattccatcaccaccatc | | |
| atcactgtagcccgggtgattga | | |
| *Region encoding Streptococcus Protein G fragment underlined | | |
| ATGACCATGATTACGGATTCACTGGCCGTCGTTTTACAACGTCGTGACTGGT | (SEQ ID NO: 52) | 30 kDa |
| ACAACCCTGGCGTTACCCAACTTAATCACCCCGTGGACTTCCAGTTCAACAT | | |
| CAGCCGCTACAGTCAACAGCAACTGATGGAAACCAGCCATCGCCATCTGCT | | |
| GCACGCGGAAGAAGGCACATGGCTGAATATCGACGGTTTCCATATGGGGAT | | |
| GGGTTACGGCCAGGACAGTCGTTTGCCGTCTGAATTTGACCTGAGCGCATT | | |
| TTTACGCGCCGGAGAAAACCGCCTCGCGGTGATGGTGCTGCGTTGGAGTG | | |
| ACGGCAGTTATCTGGAAGATCAGGATATGTGGCGGATGAGCGGCATTTTCC | | |
| GTGACGTCTCGTTGCTGCATAAACCGACTACACAAATCAGCGATTTCCATGT | | |
| TGCCACTCGCTTTAATGATGATTTCAGCCGCGCTGTACTGGAGGCTGAAGAT | | |
| GTCATGGGTGGCGACGACTCCTGGAGCCCGTCAGTATCGGCGGTATTCCA | | |
| GCTGAGCGCCGGTCGCTACCATTACCAGTTGGTCTGGTGTCAACGTGGAAC | | |
| AGATGCCGTGACAACTTACAAACTTGTTATTAATGGTAAAACATTGAAAGGC | | |
| GAAACAACTACTGAAGCTGTTGATGCTGCTACTGCAGAAAAAGTCTTCAAAC | | |
| AATACGCTAACGACAACGGTGTTGACGGTGAATGGACTTACGACGATGCGA | | |
| CTAAGACCTTTACAGTTACTGAAAAAGACATCGAATTCCATCACCACCATCAT | | |
| CACTGCAGCCCGGGTGATTGA* | | |
| ATGACCATGATTACGGATTCACTGGCCGTCGTTTTACAACGTCGTGACTGGT | (SEQ ID NO: 53) | 40 kDa |
| ACAACCCTGGCGTTACCCAACTTAATCACCCCGTGGACTTCCAGTTCAACAT | | |
| CAGCCGCTACAGTCAACAGCAACTGATGGAAACCAGCCATCGCCATCTGCT | | |
| GCACGCGGAAGAAGGCACATGGCTGAATATCGACGGTTTCCATATGGGGAT | | |
| TGGTGGCGACGACTCCTGGAGCCCGTCAGTATCGGCGGTATTCCAGCTGA | | |
| GCGGCGCAATCGAGGGTAGGGTCGCGACCATGGTTTTACGCGCCGGAGAA | | |
| AACCGCCTCGCGGTGATGGTGCTGCGTTGGAGTGACGGCAGTTATCTGGAA | | |
| GATCAGGATATGTGGCGGATGAGCGGCATTTTCCGTGACGTCTCGTTGCTG | | |
| CATAAACCGACTACACAAATCAGCGATTTCCATGTTGCCACTCGCTTTAATG | | |
| ATGATTTCAGCCGCGCTGTACTGGAGGCTGAAGFFCAGATGTGCGGCGAGT | | |
| TGCGTGACTACCTACGGGTAACAGTTTCTTTATGGCAGGGTGAAACGCAGG | | |
| TCGCCAGCGGCACCGCGCCTTTCGGCGGTGAAATTATCGATGAGCGTGGT | | |
| GGTTATGCCGATCGCGTCACACTACGTCTGAACGTCGAAAACCCGAAACTG | | |
| TGGAGCGCCGAAATCCCGAATCTCTATCGTGCGGTGGTTGAACTGCACACC | | |
| GCCGACGGCACGCTGATTGAAGCAGAAGCCTGCGATGTCGGTTTCCGCGA | | |
| GGTGCGGATTGAAAATGGTCTGCTGCTGCTGAACGGCAAGCCGTTGCTGAT | | |
| TCCCATGGAATTCGATGCGTCTGAATTAACAGATGCCTCGACAACTTACAAA | | |
| CTTGTTATTAATGGTAAAACATTGAAAGGCGGAACAACTACTGAAGCTGTTG | | |
| ATGCTGCTACTGCAGAAAAAGTCTTCAAACAATACGCtaacgacaacggtgtt | | |
| gacggtgaatggacttacgacgatgcgactaagatctttacagttactgaaaa | | |
| actgatcgaattccatcaccaccatcatcactgtagcccgggtgattga* | | |
| ATGACCATGATTACGGATTCACTGGCCGTCGTTTTACAACGTCGTGACTGGT | (SEQ ID NO: 54) | 50 kDa |
| ACAACCCTGGCGTTACCCAACTTAATCACCCCGTGGACTTCCAGTTCAACAT | | |
| CAGCCGCTACAGTCAACAGCAACTGATGGAAACCAGCCATCGCCATCTGCT | | |
| GCACGCGGAAGAAGGCACATGGCTGAATATCGACGGTTTCCATACGGCCAT | | |
| GGTTTTACGCGCCGGAGAAAACCGCCTCGCGGTGATGGTGCTGCGTTGGA | | |
| GTGACGGCAGTTATCTGGAAGATCAGGATATGTGGCGGATGAGCGGCATTT | | |
| TCCGTGACGTCTCGTTGCTGCATAAACCGACTACACAAATCAGCGATTTCCA | | |
| TGTTGCCACTCGCTTTAATGATGATTTCAGCCGCGCTGTACTGGAGGCTGAA | | |
| GTTCAGATGTGCGGCGAGTTGCGTGACTACCTACGGGTAACAGTTTCTTTAT | | |
| GGCAGGGTGAAACGCAGGTCGCCAGCGGCACCGCGCCTTTCGGCGGTGAA | | |
| ATTATCGATGAGCGTGGTGGTTATGCCGATCGCGTCACACTACGTCTGAAC | | |
| GTCGAAAACCCGAAACTGTGGAGCGCCGAAATCCCGAATCTCTATCGTGCG | | |
| GTGGTTGAACTGCACACCGCCGACGGCACGCTGATTGAAGCAGAAGCCTG | | |
| CGATGTCGGTTTCCGCGAGGTGCGGATTGAAAATGGTCTGCTGCTGCTGAA | | |
| CGGCAAGCCGTTGCTGATTCCCATGGGTTACGGCCAGGACAGTCGTTTGCC | | |
| GTCTGAATTTGACCTGAGCGCATTTTTACGCGCCGGAGAAAACCGCCTCGC | | |
| GGTGATGGTGCTGCGTTGGAGTGACGGCAGTTATCTGGAAGATCAGGATAT | | |
| GTGGCGGATGAGCGGCATTTTCCGTGACGTCTCGTTGCTGCATAAACCGAC | | |
| TACACAAATCAGCGATTTCCATGTTGCCACTCGCTTTAATGATGATTTCAGCC | | |
| GCGCTGTACTGGAGGCTGAAGATGTCATGGGTGGCGACGACTCCTGGAGC | | |
| CCGTCAGTATCGGCGGTATTCCAGCTGAGCGCCGGTCGCTACCATTACCAG | | |
| TTGGTCTGGTGTCAACGTGGAACAGATGCCGTGACAACTTACAAACTTGTTA | | |
| TTAATGGTAAAACATTGAAAGGCGAAACAACTACTGAAGCTGTTGATGCTGC | | |
| TACTGCAGAAAAAGTCTTCAAACAATACGCTAACGACAACGGTGTTGACGGT | | |
| GAATGGACTTACGACGATGCGACTAAGACCTTTACAGTTACTGAAAAAGACA | | |
| TCGAATTCCATCACCACCATCATCACTGCAGCCCGGGTGATTGA* | | |
| ATGACCATGATTACGGATTCACTGGCCGTCGTTTTACAACGTCGTGACTGGT | (SEQ ID NO: 55) | 60 kDa |
| ACAACCCTGGCGTTACCCAACTTAATCACCCCGTGGACTTCCAGTTCAACAT | | |
| CAGCCGCTACAGTCAACAGCAACTGATGGAAACCAGCCATCGCCATCTGCT | | |
| GCACGCGGAAGAAGGCACATGGCTGAATATCGACGGTTTCCATATGGGGAT | | |
| TGGTGGCGACGACTCCTGGAGCCCGTCAGTATCGGCGGTATTCCAGCTGA | | |
| GCGGCGCAATCGAGGGTAGGGTCGCGACCATGGagcagaacaactttaacgcc | | |
| gtgcgctgttcgcattatccgaaccatccgctgtggtacacgctgtgcgaccg | | |
| ctacggcctgtatgtggtggatgaagccaatattgaaacccacggcatggtgc | | |
| caatgaatcgtctgaccgatgatccgcgctggctaccggcgatgagcgaacgc | | |
| gtaacgcgaatggtgcagcgcgatcgtaatcacccgagtgtgatcatctggtc | | |
| gctggggaatgaatcaggccacggcgctaatcacgacgcgctgtatcgctgga | | |
| tcaaatctgtcgatccttcccgcccggtgcagtatgaaggcggcggagccgac | | |
| accacggccaccgatattatttgcccgatgtacgcgcgcgtggatgaagacca | | |
| gcccttcccggctgtgccgaaatggtccatcaaaaaatggctttcgctacctg | | |
| gagagacgcgcccgctgatcctttgcgaatacgcccacgcgatgggtaacagt | | |
| cttggcggtttcgctaaatactggcaggcgtttcgtcagtatccccgtttaca | | |
| gggcggcttcgtctgggactgggtggatcagtcgctgattaaatatgatgaaa | | |
| acggcaacccgtggtcggcttacggcggtgattttggcgatacgccgaacgat | | |
| cgccagttctgtatgaacggtctggtctttgccgaccgcacgccgcatccagc | | |
| gctgacggaagcaaaacaccagcagcagtttttccagttccgtttatccgggc | | |
| aaaccatcgaagtgaccagcgaatacctgttccgtcatagcgataacgagctc | | |
| ctgcactggatggtggcgctggatggtaagccgctggcaagcggtgaagtgcc | | |
| tctggatgtcgctccacaaggtaaacagttgattgaactgcctgaactaccgc | | |
| agccggagagcgccgggcaactctggctcacagtacgcgtagtgcaaccgaac | | |
| gcgaccgcatggtcagaagccgggcacatcagcgcctggcagcagtggcgtct | | |
| ggcggaaaacctcagtgtgaccatggAATTCGATGCGTCTGAATTAACAGATG | | |
| CCTCGACAACTTACAAACTTGTTATTAATGGTAAAACATTGAAAGGCGGAACA | | |
| ACTACTGAAGCTGTTGATGCTGCTACTGCAGAAAAAGTCTTCAAACAATACGC | | |
| taacgacaacggtgttgacggtgaatggacttacgacgatgcgactaagatct | | |
| ttacagttactgaaaaactgatcgaattccatcaccaccatcatcactgtagc | | |
| ccgggtgattga* | | |
| ATGACCATGATTACGGATTCACTGGCCGTCGTTTTACAACGT | (SEQ ID NO: 56) | 70 kDa |
| CGTGACTGGTACAACCCTGGCGTTACCCAACTTAATCACCC | | |
| CGTGGACTTCCAGTTCAACATCAGCCGCTACAGTCAACAGC | | |
| AACTGATGGAAACCAGCCATCGCCATCTGCTGCACGCGGAA | | |
| GAAGGCACATGGCTGAATATCGACGGTTTCCATAcggccatggag | | |
| cagaacaactttaacgccgtgcgctgttcgcattatccgaaccatccgctgtg | | |
| gtacacgctgtgcgaccgctacggcctgtatgtggtggatgaagccaatattg | | |
| aaacccacggcatggtgccaatgaatcgtctgaccgatgatccgcgctggcta | | |
| ccggcgatgagcgaacgcgtaacgcgaatggtgcagcgcgatcgtaatcaccc | | |
| gagtgtgatcatctggtcgctggggaatgaatcaggccacggcgctaatcacg | | |
| acgcgctgtatcgctggatcaaatctgtcgatccttcccgcccggtgcagtat | | |
| gaaggcggcggagccgacaccacggccaccgatattatttgcccgatgtacgc | | |
| gcgcgtggatgaagaccagcccttcccggctgtgccgaaatggtccatcaaaa | | |
| aatggctttcgctacctggagagacgcgcccgctgatcctttgcgaatacgcc | | |
| cacgcgatgggtaacagtcttggcggtttcgctaaatactggcaggcgtttcg | | |
| tcagtatccccgtttacagggcggcttcgtctgggactgggtggatcagtcgc | | |
| tgattaaatatgatgaaaacggcaacccgtggtcggcttacggcggtgatttt | | |
| ggcgatacgccgaacgatcgccagttctgtatgaacggtctggtctttgccga | | |
| ccgcacgccgcatccagcgctgacggaagcaaaacaccagcagcagtttttcc | | |
| agttccgtttatccgggcaaaccatcgaagtgaccagcgaatacctgttccgt | | |
| catagcgataacgagctcctgcactggatggtggcgctggatggtaagccgct | | |
| ggcaagcggtgaagtgcctctggatgtcgctccacaaggtaaacagttgattg | | |
| aactgcctgaactaccgcagccggagagcgccgggcaactctggctcacagta | | |
| cgcgtagtgcaaccgaacgcgaccgcatggtcagaagccgggcacatcagcgc | | |
| ctggcagcagtggcgtctggcggaaaacctcagtgtgaccatggGTTACGGCC | | |
| AGGACAGTCGTTTGCCGTCTGAATTTGACCTGAGCGCATTTTTACGCGCCGGA | | |
| GAAAACCGCCTCGCGGTGATGGTGCTGCGTTGGAGTGACGGCAGTTATCTGGA | | |
| AGATCAGGATATGTGGCGGATGAGCGGCATTTTCCGTGACGTCTCGTTGCTGC | | |
| ATAAACCGACTACACAAATCAGCGATTTCCATGTTGCCACTCGCTTTAATGAT | | |
| GATTTCAGCCGCGCTGTACTGGAGGCTGAAGATGTCATGGGTGGCGACGACTC | | |
| CTGGAGCCCGTCAGTATCGGCGGTATTCCAGCTGAGCGCCGGTCGCTACCATT | | |
| ACCAGTTGGTCTGGTGTCAACGTGGAACAGATGCCGTGACAACTTACAAACTT | | |
| GTTATTAATGGTAAAACATTGAAAGGCGAAACAACTACTGAAGCTGTTGATGC | | |
| TGCTACTGCAGAAAAAGTCTTCAAACAATACGCTAACGACAACGGTGTTGACG | | |
| GTGAATGGACTTACGACGATGCGACTAAGACCTTTACAGTTACTGAAAAAGAC | | |
| ATCGAATTCCATCACCACCATCATCACTGCAGCCCGGGTGATTGACACTGCAG | | |
| CCCGGGTGATTGA | | |
| ATGACCATGATTACGGATTCACTGGCCGTCGTTFTACAACGTCGTGACTGGT | (SEQ ID NO: 57) | 80 kDa |
| ACAACCCTGGCGTTACCCAACTTAATCACCCCGTGGACTTCCAGTTCAACAT | | |
| CAGCCGCTACAGTCAACAGCAACTGATGGAAACCAGCCATCGCCATCTGCT | | |
| GCACGCGGAAGAAGGCACATGGCTGAATATCGACGGTTTCCATACGGCCAT | | |
| GGAGCAGAACAACTTTAACGCCGTGCGCTGTTCGCATTATCCGAACCATCC | | |
| GCTGTGGTACACGCTGTGCGACCGCTACGGCCTGTATGTGGTGGATGAAGC | | |
| CAATATTGAAACCCACGGCATGGTGCCAATGAATCGTCTGACCGATGATCC | | |
| GCGCTGGCTACCGGCGATGAGCGAACGCGTAACGCGAATGGTGCAGCGCG | | |
| ATCGTAATCACCCGAGTGTGATCATCTGGTCGCTGGGGAATGAATCAGGCC | | |
| ACGGCGCTAATCACGACGCGCTGTATCGCTGGATCAAATCTGTCGATCCTT | | |
| CCCGCCCGGTGCAGTATGAAGGCGGCGGAGCCGACACCACGGCCACCGAT | | |
| ATTATTTGCCCGATGTACGCGCGCGTGGATGAAGACCAGCCCTTCCCGGCT | | |
| GTGCCGAAATGGTCCATCAAAAAATGGCTTTCGCTACCTGGAGAGACGCGC | | |
| CCGCTGATCCTTTGCGAATACGCCCACGCGATGGGTAACAGTCTTGGCGGT | | |
| TTCGCTAAATACTGGCAGGCGTTTCGTCAGTATCCCCGTTTACAGGGCGGC | | |
| TTCGTCTGGGACTGGGTGGATCAGTCGCTGATTAAATATGATGAAAACGGC | | |
| AACCCGTGGTCGGCTTACGGCGGTGATTTTGGCGATACGCCGAACGATCGC | | |
| CAGTTCTGTATGAACGGTCTGGTCTTTGCCGACCGCACGCCGCATCCAGCG | | |
| CTGACGGAAGCAAAACACCAGCAGCAGTTTTTCCAGTTCCGTTTATCCGGG | | |
| CAAACCATCGAAGTGACCAGCGAATACCTGTTCCGTCATAGCGATAACGAG | | |
| CTCCTGCACTGGATGGTGGCGCTGGATGGTAAGCCGCTGGCAAGCGGTGA | | |
| AGTGCCTCTGGATGTCGCTCCACAAGGTAAACAGTTGATTGAACTGCCTGAA | | |
| CTACCGCAGCCGGAGAGCGCCGGGCAACTCTGGCTCACAGTACGCGTAGT | | |
| GCAACCGAACGCGACCGCATGGTCAGAAGCCGGGCACATCAGCGCCTGGC | | |
| AGCAGTGGCGTCTGGCGGAAAACCTCAGTGTGACGCTCCCCGCCGCGTCC | | |
| CACGCCATCCCGCATCTGACCACCAGCGAAATGGATTTTTGCATCGAGCTG | | |
| GGTAATAAGCGTTGGCAATTTAACCGCCAGTCAGGCTTTCTTTCACAGATGT | | |
| GGATTGGCGATAAAAAACAACTGCTGACGCCGCTGCGCGATCAGTTCACCC | | |
| GTGCACCGCTGGATAACGACATTGGCGTAAGTGAAGCGACCCGCATTGACC | | |
| CTAACGCCTGGGTCGAACGCTGGAAGGCGGCGGGCCATTACCAGGCCGAA | | |
| GCAGCGTTGTTGCAGTGCACGGCAGATACACTTGCTGATGCGGTGCTGATT | | |
| ACGACCGCTCACGCGTGGCAGCATCAGGGGAAAACCTTATTTATCAGCCGG | | |
| AAAACCTACCGGATTGATGGTAGTGGTCAAATGGCGATTACCGTTGATGTTG | | |
| AAGTGGCGAGCGATACACCGCATCCGGCGCGGATTGGCCTGAACTGCCAG | | |
| CTGGCGCAGGTAGCAGAGCGGGTAAACTGGCTCGGATTAGGGCCGCAAGA | | |
| AAACTATCCCGGCCCCATGGGTGGCGACGACTCCTGGAGCCCGTCAGTATC | | |
| GGCGGTATTCCAGCTGAGCGCCGGTCGCTACCATTACCAGTTGGTCTGGTG | | |
| TCAACGTGGAACAGATGCCGTGACAACTTACAAACTTGTTATTAATGGTAAA | | |
| ACATTGAAAGGCGAAACAACTACTGAAGCTGTTGATGCTGCTACTGCAGAAA | | |
| AAGTCTTCAAACAATACGCTAACGACAACGGTGTTGACGGTGAATGGACTTA | | |
| CGACGATGCGACTAAGACCTTTACAGTTACTGAAAAAGACATCGAATTCCAT | | |
| CACCACCATCATCACTGCAGCCCGGGTGATTGA | | |
| ATGACCATGATTACGGATTCACTGGCCGTCGTTTTACAACGTCGTGACTGGT | (SEQ ID NO: 58) | 100 kDa |
| ACAACCCTGGCGTTACCCAACTTAATCACCCCGTGGACTTCCAGTTCAACAT | | |
| CAGCCGCTACAGTCAACAGCAACTGATGGAAACCAGCCATCGCCATCTGCT | | |
| GCACGCGGAAGAAGGCACATGGCTGAATATCGACGGTTTCCATATGGGGAT | | |
| TGGTGGCGACGACTCCTGGAGCCCGTCAGTATCGGCGGTATTCCAGCTGA | | |
| GCGGCGCAATCGAGGGTAGGGTCGCGACCATGGGTTACGGCCAGGACAGT | | |
| CGTTTGCCGTCTGAATTTGACCTGAGCGCATTTTTACGCGCCGGAGAAAAC | | |
| CGCCTCGCGGTGATGGTGCTGCGTTGGAGTGACGGCAGTTATCTGGAAGAT | | |
| CAGGATATGTGGCGGATGAGCGGCATTTTCCGTGACGTCTCGTTGCTGCAT | | |
| AAACCGACTACACAAATCAGCGATTTCCATGTTGCCACTCGCTTTAATGATG | | |
| ATTTCAGCCGCGCTGTACTGGAGGCTGAAGTTCAGATGTGCGGCGAGTTGC | | |
| GTGACTACCTACGGGTAACAGTTTCTTTATGGCAGGGTGAAACGCAGGTCG | | |
| CCAGCGGCACCGCGCCTTTCGGCGGTGAAATTATCGATGAGCGTGGTGGTT | | |
| ATGCCGATCGCGTCACACTACGTCTGAACGTCGAAAACCCGAAACTGTGGA | | |
| GCGCCGAAATCCCGAATCTCTATCGTGCGGTGGTTGAACTGCACACCGCCG | | |
| ACGGCACGCTGATTGAAGCAGAAGCCTGCGATGTCGGTTTCCGCGAGGTG | | |
| CGGATTGAAAATGGTCTGCTGCTGCTGAACGGCAAGCCGTTGCTGATTCGA | | |
| GGCGTTAACCGTCACGAGCATCATCCTCTGCATGGTCAGGTCATGGATGAG | | |
| CAGACGATGGTGCAGGATATCCTGCTGATGAAGCAGAACAACTTTAACGCC | | |
| GTGCGCTGTTCGCATTATCCGAACCATCCGCTGTGGTACACGCTGTGCGAC | | |
| CGCTACGGCCTGTATGTGGTGGATGAAGCCAATATTGAAACCCACGGCATG | | |
| GTGCCAATGAATCGTCTGACCGATGATCCGCGCTGGCTACCGGCGATGAGC | | |
| GAACGCGTAACGCGAATGGTGCAGCGCGATCGTAATCACCCGAGTGTGATC | | |
| ATCTGGTCGCTGGGGAATGAATCAGGCCACGGCGCTAATCACGACGCGCT | | |
| GTATCGCTGGATCAAATCTGTCGATCCTTCCCGCCCGGTGCAGTATGAAGG | | |
| CGGCGGAGCCGACACCACGGCCACCGATATTATTTGCCCGATGTACGCGC | | |
| GCGTGGATGAAGACCAGCCCTTCCCGGCTGTGCCGAAATGGTCCATCAAAA | | |
| AATGGCTTTCGCTACCTGGAGAGACGCGCCCGCTGATCCTTTGCGAATACG | | |
| CCCACGCGATGGGTAACAGTCTTGGCGGTTTCGCTAAATACTGGCAGGCGT | | |
| TTCGTCAGTATCCCCGTTTACAGGGCGGCTTCGTCTGGGACTGGGTGGATC | | |
| AGTCGCTGATTAAATATGATGAAAACGGCAACCCGTGGTCGGCTTACGGCG | | |
| GTGATTTTGGCGATACGCCGAACGATCGCCAGTTCTGTATGAACGGTCTGG | | |
| TCTTTGCCGACCGCACGCCGCATCCAGCGCTGACGGAAGCAAAACACCAG | | |
| CAGCAGTTTTTCCAGTTCCGTTTATCCGGGCAAACCATCGAAGTGACCAGC | | |
| GAATACCTGTTCCGTCATAGCGATAACGAGCTCCTGCACTGGATGGTGGCG | | |
| CTGGATGGTAAGCCGCTGGCAAGCGGTGAAGTGCCTCTGGATGTCGCTCCA | | |
| CAAGGTAAACAGTTGATTGAACTGCCTGAACTACCGCAGCCGGAGAGCGCC | | |
| GGGCAACTCTGGCTCACAGTACGCGTAGTGCAACCGAACGCGACCGCATG | | |
| GTCAGAAGCCGGGCACATCAGCGCCTGGCAGCAGTGGCGTCTGGCGGAAA | | |
| ACCTCAGTGTGACGCTCCCCGCCGCGTCCCACGCCATCCCGCATCTGACCA | | |
| CCAGCGAAATGGATTTTTGCATCGAGCTGGGTAATAAGCGTTGGCAATTTAA | | |
| CCGCCAGTCAGGCTTTCTTTCACAGATGTGGATTGGCGATAAAAAACAACTG | | |
| CTGACGCCGCTGCGCGATCAGTTCACCCGTGCACCGCTGGATAACGACATT | | |
| GGCGTAAGTGAAGCGACCCGCATTGACCCTAACGCCTGGGTCGAACGCTG | | |
| GAAGGCGGCGGGCCATTACCAGGCCGAAGCAGCGTTGTTGCAGTGCACGG | | |
| CAGATACACTTGCTGATGCGGTGCTGATTACGACCGCTCACGCGTGGCAGC | | |
| ATCAGGGGAAAACCTTATTTATCAGCCGGAAAACCTACCGGATTGATGGTAG | | |
| TGGTCCATCCATGGAATTCGATGCGTCTGAATTAACAGATGCCTCGACAACT | | |
| TACAAACTTGTTATTAATGGTAAAACATTGAAAGGCGGAACAACTACTGAAG | | |
| CTGTTGATGCTGCTACTGCAGAAAAAGTCTTCAAACAATACGCtaacgacaacg | | |
| gtgttgacggtgaatggacttacgacgatgcgactaagatctttacagttactg | | |
| aaaaactgatcgaattccatcaccaccatcatcactgtagcccgggtgattga* | | |
| ATGACCATGATTACGGATTCACTGGCCGTCGTTTTACAACGTCGTGACTGGTACA | (SEQ ID NO: 59) | 120 kDa |
| ACCCTGGCGTTACCCAACTTAATCACCCCGTGGACTTCCAGTTCAACATCAGCC | | |
| GCTACAGTCAACAGCAACTGATGGAAACCAGCCATCGCCATCTGCTGCACGCGG | | |
| AAGAAGGCACATGGCTGAATATCGACGGTTTCCATATGGGGATTGGTGGCGACG | | |
| ACTCCTGGAGCCCGTCAGTATCGGCGGTATTCCAGCTGAGCGGCGCAATCGAG | | |
| GGTAGGGTCGCGACCATGGGTTACGGCCAGGACAGTCGTTTGCCGTCTGAATTT | | |
| GACCTGAGCGCATTTTTTACGCGCCGGAGAAAACCGCCTCGCGGTGATGGTGCT | | |
| GCGTTGGAGTGACGGCAGTTATCTGGAAGATCAGGATATGTGGCGGATGAGCG | | |
| GCATTTTCCGTGACGTCTCGTTGCTGCATAAACCGACTACACAAATCAGCGATTT | | |
| CCATGTTGCCACTCGCTTTAATGATGATTTCAGCCGCGCTGTACTGGAGGCTGAA | | |
| GTTCAGATGTGCGGCGAGTTGCGTGACTACCTACGGGTAACAGTTTCTTTATGG | | |
| CAGGGTGAAACGCAGGTCGCCAGCGGCACCGCGCCTTTCGGCGGTGAAATTAT | | |
| CGATGAGCGTGGTGGTTATGCCGATCGCGTCACACTACGTCTGAACGTCGAAAA | | |
| CCCGAAACTGTGGAGCGCCGAAATCCCGAATCTCTATCGTGCGGTGGTTGAACT | | |
| GCACACCGCCGACGGCACGCTGATTGAAGCAGAAGCCTGCGATGTCGGTTTCC | | |
| GCGAGGTGCGGATTGAAAATGGTCTGCTGCTGCTGAACGGCAAGCCGTTGCTGA | | |
| TTCGAGGCGTTAACCGTCACGAGCATCATCCTCTGCATGGTCAGGTCATGGATG | | |
| AGCAGACGATGGTGCAGGATATCCTGCTGATGAAGCAGAACAACTTTAACGCCG | | |
| TGCGCTGTTCGCATTATCCGAACCATCCGCTGTGGTACACGCTGTGCGACCGCT | | |
| ACGGCCTGTATGTGGTGGATGAAGCCAATATTGAAACCCACGGCATGGTGCCAA | | |
| TGAATCGTCTGACCGATGATCCGCGCTGGCTACCGGCGATGAGCGAACGCGTA | | |
| ACGCGAATGGTGCAGCGCGATCGTAATCACCCGAGTGTGATCATCTGGTCGCTG | | |
| GGGAATGAATCAGGCCACGGCGCTAATCACGACGCGCTGTATCGCTGGATCAAA | | |
| TCTGTCGATCCTTCCCGCCCGGTGCAGTATGAAGGCGGCGGAGCCGACACCAC | | |
| GGCCACCGATATTATTTGCCCGATGTACGCGCGCGTGGATGAAGACCAGCCCTT | | |
| CCCGGCTGTGCCGAAATGGTCCATCAAAAAATGGCTTTCGCTACCTGGAGAGAC | | |
| GCGCCCGCTGATCCTTTGCGAATACGCCCACGCGATGGGTAACAGTCTTGGCG | | |
| GTTTCGCTAAATACTGGCAGGCGTTTCGTCAGTATCCCCGTTTACAGGGCGGCTT | | |
| CGTCTGGGACTGGGTGGATCAGTCGCTGATTAAATATGATGAAAACGGCAACCC | | |
| GTGGTCGGCTTACGGCGGTGATTTTGGCGATACGCCGAACGATCGCCAGTTCTG | | |
| TATGAACGGTCTGGTCTTTGCCGACCGCACGCCGCATCCAGCGCTGACGGAAG | | |
| CAAAACACCAGCAGCAGTTTTTCCAGTTCCGTTTATCCGGGCAAACCATCGAAGT | | |
| GACCAGCGAATACCTGTTCCGTCATAGCGATAACGAGCTCCTGCACTGGATGGT | | |
| GGCGCTGGATGGTAAGCCGCTGGCAAGCGGTGAAGTGCCTCTGGATGTCGCTC | | |
| CACAAGGTAAACAGTTGATTGAACTGCCTGAACTACCGCAGCCGGAGAGCGCCG | | |
| GGCAACTCTGGCTCACAGTACGCGTAGTGCAACCGAACGCGACCGCATGGTCA | | |
| GAAGCCGGGCACATCAGCGCCTGGCAGCAGTGGCGTCTGGCGGAAAACCTCAG | | |
| TGTGACGCTCCCCGCCGCGTCCCACGCCATCCCGCATCTGACCACCAGCGAAA | | |
| TGGATTTTTGCATCGAGCTGGGTAATAAGCGTTGGCAATTTAACCGCCAGTCAGG | | |
| CTTTCTTTCACAGATGTGGATTGGCGATAAAAAACAACTGCTGACGCCGCTGCGC | | |
| GATCAGTTCACCCGTGCACCGCTGGATAACGACATTGGCGTAAGTGAAGCGACC | | |
| CGCATTGACCCTAACGCCTGGGTCGAACGCTGGAAGGCGGCGGGCCATTACCA | | |
| GGCCGAAGCAGCGTTGTTGCAGTGCACGGCAGATACACTTGCTGATGCGGTGCT | | |
| GATTACGACCGCTCACGCGTGGCAGCATCAGGGGAAAACCTTATTTATCAGCCG | | |
| GAAAACCTACCGGATTGATGGTAGTGGTCAAATGGCGATTTACCGTTTGATGTTG | | |
| AAGTGGCGAGCGATACACCGCATCCGGCGCGGATTGGCCTGAACTGCCAGCTGGC | | |
| GCAGGTAGCAGAGCGGGTAAACTGGCTCGGATTAGGGCCGCAAGAAAACTATC | | |
| CCGACCGCCTTACTGCCGCCTGTTTTGACCGCTGGGATCTGCCATTGTCAGACA | | |
| TGTATACCCCGTACGTCTTCCCGAGCGAAAACGGTCTGCGCTGCGGGACGCGC | | |
| GAATTGAATTATGGCCCACACCAGTGGCGCGGCGACTTCCAGTTCAACATCAGC | | |
| CGCTACAGTCAACAGCAACTGATGGAAACCAGCCATCGCCATCTGCTGCACGCG | | |
| GAAGAAGGCACATGGCTGAATATCGACGGTTTCCATATGGGGATTGGTGGCGAC | | |
| GACTCCTGGAGCCCGTCAGTATCGGCGGTATTCCAGCTGAGCGCCGGTCGCTA | | |
| CCATTACCAGTTGGTCTGGTGTCAAAAAGGGGATCCTGGTCCTGTTGGTCCTGTT | | |
| GGTCCTGCTGGTGCTTTTGGCCCAAGAGGTGCCGCCATGGAATTCGATGCGTCT | | |
| GAATTAACAGATGCCTCGACAACTTACAAACTTGTTATTAATGGTAAAACATTGA | | |
| AAGGCGGAACAACTACTGAAGCTGTTGATGCTGCTACTGCAGAAAAAGTCTTCAA | | |
| ACAATACGCtaacgacaacggtgttgacggtgaatggacttacgacgatgcgact | | |
| aagatctttacagttactgaaaaactgatcgaattccatcaccaccatcatcact | | |
| gtagcccgggtgattga* | | |

TABLE 7
Amino Acid Sequence of the 40, 60 and 100 kDa protein fragments

| | | |
|---|---|---|
| 40 kDa | Mtmitdslavvlqrrdwynpgvtqlnhpvdfqfnisrysqqqlmetshrhllhaeegtwlnidgfhmgiggddsws | (SEQ ID NO: 60) |
| protein | psvsavfqlsgaiegrvatmvlragenrlavmvlrwsdgsyledqdmwrmsgifrdvsllhkpttqisdfhvatrf | |
| | nddfsravleaevqmcgelrdylrvtvslwqgetqvasgtapfggeiiderggyadrvtlrlnvenpklwsaeipn | |
| | lyravvelhtadgtlieaeacdvgfrevriengllllngkpllipmefdaseltdasttyklvingktlkggttte | |
| | avdaataekvfkgyandngvdgewtyddatkiftvtekliefhhhhhhcspgd | |
| | *Streptococccus Protein G segment is underlined | |
| 60 kDa | Mtmitdslavvlqrrdwynpgvtqlnhpvdfqfnisrysqqqlmetshrhllhaeegtwlnidgfhmgiggddsws | (SEQ ID NO: 61) |
| protein | psvsavfqlsgaiegrvatmeqnnfnavrcshypnhplwytlcdryglyvvdeaniethgmvpmnrltddprwlp | |
| | amservtrmvqrdrnhpsviiwslgnesghganhdalyrwiksvdpsrpvqyegggadttatdiicpmyarvded | |
| | qpfpavpkwsikkwlslpgetrplilceyahamgnslggfakywqafrqyprlqggfvwdwvdqslikydengnp | |
| | wsayggdfgdtpndrqfcmnglvfadrtphpalteakhqqqffqfrlsgqtievtseylfrhsdnellhwmvaldg | |
| | kplasgevpldvapqgkqlielpelpqpesagqlwltvrvvqpnatawseaghisawqqwrlaenlsvtmefdase | |
| | ltdasttyklvingktlkggttteavdaataekvfkgyandngvdgewtyddatkiftvtekliefhhhhhhcspgd | |
| | *Streptococccus Protein G segment is underlined | |
| 100 kDa | Mtmitdslavvlqrrdwynpgvtqlnhpvdfqfnisrysqqqlmetshrhllhaeegtwlnidgfhmgiggddsws | (SEQ ID NO: 62) |
| protein | psvsavfqlsgaiegrvatmgygqdsrlpsefdisaflragenriavmvlrwsdgsyledqdmwrmsgifrdvsllh | |
| | kpttqisdfhvatrfnddfsravleaevqmcgelrdylrvtvslwqgetqvasgtapfggeiiderggyadrvtlrl | |
| | nvenpklwsaeipnlyravvelhtadgtlieaeacdvgfrevriengllllngkpllirgvnrhehhplhgqvmdeq | |
| | tmvqdillmkqnnfnavrcshypnhplwytlcdryglyvvdeaniethgmvpmnrltddprwlpamservtrmvqrd | |
| | mhpsviiwslgnesghganhdalyrwiksvdpsrpvqyegggadttatdiicpmyarvdedqpfpavpkwsikkwls | |
| | lpgetrplilceyahamgnslggfakywqafrqyprlqggfvwdwvdqslikydengnpwsayggdfgdtpndrqfc | |
| | mnglvfadrtphpalteakhqqqffqfrlsgqtievtseylfrhsdnellhwmvaldgkplasgevpldvapqgkql | |
| | ieipelpqpesagqlwltvrvvqpnatawseaghisawqqwrlaenlsvtlpaashaiphlttsemdfcielgnkrw | |
| | qfnrqsgflsqmwigdkkqlltplrdqftrapldndigvseatridpnawverwkaaghyqaeaallqctadtlada | |
| | vlittahawqhqgktlfisrktyridgsgpsmefdaseltdasttyklvingktlkaattteavdaataekvfkgya | |
| | ndngvdgewtyddatkiftvtekliefhhhhhhcspgd | |
| | *Streptococecus Protein G segment is underlined | |

1. A method of preparing a product nucleic acid molecule having a known or predetermined physical characteristic comprising:
(a) providing at least two starting nucleic acid molecules, wherein at least one nucleic acid molecule comprises a segment having a known or predetermined physical characteristic and wherein each nucleic acid molecule comprises at least one recombination site capable of recombining with a recombination site present on another segment or another starting nucleic acid molecule;
(b) contacting said nucleic acid molecules under conditions causing recombination between the recombination sites, thereby joining said nucleic acid molecules, or segments thereof, and producing a product nucleic acid molecule; and
(c) isolating said product nucleic acid molecule.

2. The method of claim 1, wherein two, three, four, five, six, seven or eight starting nucleic acid molecules are provided.

3. The method of claim 1, wherein at least one starting nucleic acid molecule comprises a region capable of binding a protein.

4. The method of claim 1, wherein said physical characteristic is length in bp.

5. A composition comprising the product nucleic acid molecule of claim 1.

15. A method of preparing a protein marker molecule comprising:
(a) providing at least two starting nucleic acid molecules, wherein at least one nucleic acid molecule comprises a segment encoding a protein having a known or predetermined physical characteristic and wherein each nucleic acid molecule comprises at least one recombination site capable of recombining with a recombination site present on another segment or starting nucleic acid;
(b) contacting said starting nucleic acid molecules under conditions causing recombination between the recombination sites, thereby joining said nucleic acid molecules, or said segments, and producing a product nucleic acid molecule which encodes a protein marker molecule;
(c) transforming said product nucleic acid molecule into a host cell;
(d) causing the product nucleic molecule of (c) to express the encoded protein; and
(e) purifying said expressed protein marker molecule.

16. (Cancelled)

17. (Cancelled)

18. An isolated polypeptide having the structure:
N-(-[M]ₙ-)-C
wherein:
N is an amino terminus;
C is a carboxy terminus;
- represents any number, including 0, of amino acids arranged in any order;
M is an amino acid sequence comprising a domain dM; and
n is any whole integer.

19. The polypeptide of claim 18, wherein n is any integer greater than 2.

20. (Cancelled)

21. The polypeptide of claim 18, wherein dM is selected from the group consisting of an enzymatic domain, a binding domain and a detectable domain.

22. The polypeptide of claim 21, wherein said domain dM is an enzymatic domain.

23. The polypeptide of claim 22, wherein said enzymatic domain is from an enzyme selected from the group consisting of a nuclease, a recombinase, a phosphatase, and a kinase.

24. The polypeptide of claim 21, wherein said domain dM is a binding domain.

25. The polypeptide of claim 24 wherein said binding domain is selected from the group consisting of a CDR, a camel antibody, a single-chain antibody, a domain that binds the constant region of an antibody, a domain that binds a nucleic acid, and a domain that is a ligand of a receptor.

26. The polypeptide of claim 25, wherein said binding domain is an IgG binding domain.

27. The polypeptide of claim 21, wherein said binding domain binds a nucleic acid.

28. The polypeptide of claim 27, wherein said nucleic acid is selected from the group consisting of an RNA, tRNA, a ribosomal RNA, an mRNA, an antisense nucleic acid, a DNA, a site that regulates gene expression, a nucleic acid from a pathogen, and a nucleic acid from a sample

29. The polypeptide of claim 21, wherein said binding domain binds a substance selected from the group consisting of a lipid; a small organic molecule; biotin or a biotinylated compound; a cell surface antigen or receptor; a crystal; and an artificial polymer.

30. The polypeptide of claim 21, wherein said domain dM is a detectable domain.

31-35. (Cancelled)

36. A kit comprising a container containing a polypeptide according to claim 18.

37. A kit comprising a nucleic acid molecule encoding a polypeptide according to claim 18.

38. (Cancelled)
